(19) United States
(12) Patent Application Publication
(10) Pub. No.: US 2025/0250261 A1
(43) Pub. Date: Aug. 7, 2025

(54) NOVEL SUBSTITUTED FLUORINATED VINYL-N-PROPYL-PYRROLIDINE COMPOUNDS, PROCESSES FOR THEIR PREPARATION AND THERAPEUTIC USES THEREOF

(71) Applicant: SANOFI, Paris (FR)

(72) Inventors: Patrick BERNARDELLI, Paris (FR); Youssef EL-AHMAD, Paris (FR); Franck SLOWINSKI, Paris (FR)

(21) Appl. No.: 18/856,557

(22) PCT Filed: Apr. 14, 2023
(86) PCT No.: PCT/EP2023/059813

§ 371 Date: Oct. 11, 2024

(30) Foreign Application Priority Data
Apr. 15, 2022 (EP) 22305563.3

(51) Int. Cl. C07D 409/10; C07D 207/20

(52) U.S. Cl. CPC C07D 409/10; C07D 207/20

(57) ABSTRACT

Disclosed herein are compounds of the formula (I), or pharmaceutically acceptable salts thereof, wherein R1 and R2 represent hydrogen or deuterium; R3 represents hydrogen, —COOH or —OH; R3′ and R3″ represent hydrogen, methyl, methoxy, chlorine, fluorine or cyano; R4 and R4′ represent hydrogen or fluorine; R5 represents hydrogen, fluorine or (C₁-C₃)alkyl; R6 represents phenyl, fused phenyl, bicyclic group comprising 5 to 12 carbon atoms, heteroaryl group comprising 2 to 9 carbon atoms and comprising from 1 to 3 heteroatoms, cycloalkyl group comprising 3 to 7 carbon atoms, (C₃-C₆)cycloalkyl(C₁-C₃)alkyl group, 4 to 7 membered-heterocycloalkyl group comprising 1 or 2 heteroatoms, (C₁-C₆)alkyl, and phenyl(C₁-C₂)alkyl group; X represents —CH₂—, —O— or —S—; Y represents —CH═, —N═ or —CR″═, wherein R″ represents (C₁-C₃)alkyl, halogen, cyano, or (C₁-C₃)fluoroalkyl; R7 represents (C₁-C₃)alkyl, halogen atom, cyano, or (C₁-C₃)fluoroalkyl; R8 represents hydrogen or fluorine; R9 represents hydrogen, (C₁-C₃)alkyl or a cyclopropyl; n is 0, 1 or 2; and m is 0 or 1. Further disclosed are process for preparing the same, pharmaceutical compositions comprising them as well as said compounds of formula (I) for use as an inhibitor and degrader of estrogen receptors, in particular in the treatment of ovulatory dysfunction, cancer, endometriosis, osteoporosis, benign prostatic hypertrophy or inflammation.

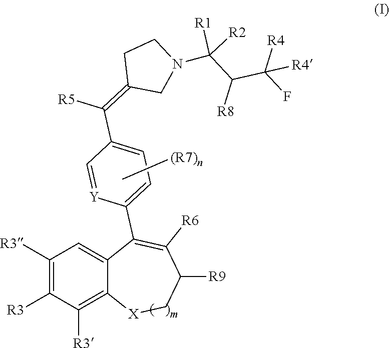

Disclosed herein are novel fluorinated vinyl-N-propyl-pyrrolidine derivatives, the processes for their preparation, as well as the therapeutic uses thereof, in particular as anticancer agents via selective antagonism and degradation of estrogen receptors.

The Estrogen Receptors (ER) belong to the steroid/nuclear receptor superfamily involved in the regulation of eukaryotic gene expression, cellular proliferation and differentiation in target tissues. ERs are in two forms: the estrogen receptor alpha (ERa) and the estrogen receptor beta (ERP) respectively encoded by the ESR1 and the ESR2 genes. ERα and ERβ are ligand-activated transcription factors which are activated by the hormone estrogen (the most potent estrogen produced in the body is 17β-estradiol). In the absence of hormone, ERs are largely located in the cytosol of the cell. When the hormone estrogen binds to ERs, ERs migrate from the cytosol to the nucleus of the cell, form dimers and then bind to specific genomic sequences called Estrogen Response Elements (ERE). The DNA/ER complex interacts with co-regulators to modulate the transcription of target genes.

ERα is mainly expressed in reproductive tissues such as uterus, ovary, breast, bone and white adipose tissue. Abnormal ERα signaling leads to development of a variety of diseases, such as cancers, metabolic and cardiovascular diseases, neurodegenerative diseases, inflammation diseases and osteoporosis.

ERα is expressed in not more than 10% of normal breast epithelium but approximately 50-80% of breast tumors. Such breast tumors with high level of ERα are classified as ERa-positive breast tumors. The etiological role of estrogen in breast cancer is well established and modulation of ERα signaling remains the mainstay of breast cancer treatment for the majority ERα-positive breast tumors. Currently, several strategies for inhibiting the estrogen axis in breast cancer exist, including: 1—blocking estrogen synthesis by aromatase inhibitors that are used to treat early and advanced ERα-positive breast cancer patients; 2—antagonizing estrogen ligand binding to ERα by tamoxifen which is used to treat ERα-positive breast cancer patients in both pre- and post-menopausal setting; 3—antagonizing and downregulating ERα levels by fulvestrant, which is used to treat breast cancer in patients that have progressed despite endocrine therapies such as tamoxifen or aromatase inhibitors.

Although these endocrine therapies have contributed enormously to reduction in breast cancer development, about more than one-third of ERα-positive patients display de novo resistance or develop resistance over time to such existing therapies. Several mechanisms have been described to explain resistance to such hormone therapies. For example, hypersensitivity of ERα to low estrogen level in treatment with aromatase inhibitors, the switch of tamoxifen effects from antagonist to agonist effects in tamoxifen treatments or multiple growth factor receptor signaling pathways. Acquired mutations in ERα occurring after initiation of hormone therapies may also play a role in treatment failure and cancer progression. Certain mutations in ERα, particularly those identified in the Ligand Binding Domain (LBD), result in the ability to bind to DNA in the absence of ligand and confer hormone independence in cells harboring such mutant receptors.

Most of the endocrine therapy resistance mechanisms identified rely on ERα-dependent activity. One of the new strategies to counterforce such resistance is to shut down the ERα signaling by removing ERα from the tumor cells using Selective Estrogen Receptors Degraders (SERDs). Clinical and preclinical data showed that a significant number of the resistance pathways can be circumvented by the use of SERDs.

There is still a need to provide SERDs with good degradation efficacy.

Documents WO2017/140669 and WO2018/091153 disclose some substituted 6,7-dihydro-5H-benzo[7]annulene compounds and substituted N-(3-fluoropropyl)-pyrrolidine derivatives useful as SERDs.

The inventors have now found novel compounds able to selectively antagonize and degrade the estrogen receptors (SERDs compounds), for use in cancer treatment.

Disclosed herein are compounds of the formula (I), or pharmaceutically acceptable salts thereof:

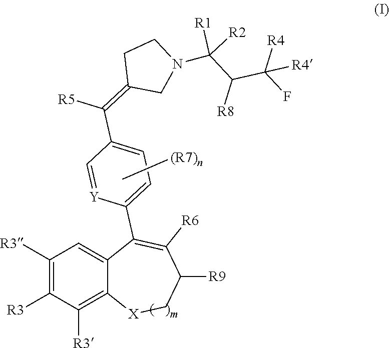

wherein:
   R1 and R2 independently represent a hydrogen atom or a deuterium atom;
   R3 represents a hydrogen atom, a —COOH group or a —OH group;
   R3′ and R3″ independently represent a hydrogen atom, a methyl group, a methoxy group, a chlorine atom, a fluorine atom or a cyano group;
   R4 and R4′ independently represent a hydrogen atom or a fluorine atom;
   R5 represents a hydrogen atom, a fluorine atom or a (C₁-C₃)alkyl group;
   R6 represents a group selected from:
      a phenyl group, said phenyl group being optionally substituted by 1 to 3 substituents independently selected from a halogen atom; a (C₁-C₆)alkyl group optionally substituted with a cyano group or a —OH group; a (C₁-C₆)fluoroalkyl group; a (C₃-C₆)cycloalkyl group; a (C₁-C₆)alkoxy group; a (C₁-C₆)fluoroalkoxy group; a cyano group; a trifluoromethylsulfonyl group; a (C₁-C₄)alkylthio group; a (C₁-C₄)fluoroalkylthio group; a (C₁-C₄)alkylsulfonyl group and a —OH group;
      a fused phenyl group, selected from phenyl groups fused with a (C₃-C₆)cycloalkyl, said (C₃-C₆)cycloalkyl optionally comprises an unsaturation, and wherein the fused phenyl group is optionally substituted with 1 to 3 substituents independently selected from a (C₁-C₃)alkyl group, a hydroxy group, a halogen atom, a (C₁-C₆)fluoroalkyl group and a (C₁-C₃)alkoxy group;
      a bicyclic group comprising 5 to 12 carbon atoms, optionally comprising 1 to 2 unsaturations; optionally substituted with 1 to 4 substituents independently selected from: a fluorine atom, a —OH group, a (C₁-C₃)alkyl group, a (C₁-C₃)fluoroalkyl group, a (C₁-C₃)alkoxy group, a (C₁-C₃)fluoroalkoxy group and an oxo group;
      a heteroaryl group comprising 2 to 9 carbon atoms and comprising from 1 to 3 heteroatoms independently selected from oxygen, nitrogen and sulfur, and at least 5 atoms including carbon atoms and heteroatoms, such as a pyridyl group, said heteroaryl group being optionally substituted with 1 to 3 substituents independently selected from a halogen atom, a (C₁-C₆)alkyl group, a (C₁-C₆)fluoroalkyl group, a (C₁-C₆)alkoxy group, a (C₁-C₆)fluoroalkoxy group, a cyano group, a carbamoyl group and a —OH group;
      a cycloalkyl group comprising 3 to 7 carbon atoms, said cycloalkyl group being saturated or partially saturated and being optionally substituted with 1 to 4 substituents independently selected from:
         a fluorine atom, a —OH group, a (C₁-C₃)alkyl group, a (C₁-C₃)fluoroalkyl group, a (C₁-C₃)alkoxy group, a (C₁-C₃)fluoroalkoxy group, an oxo group,
         a (C₃-C₆)cycloalkyl group and a phenyl group, said (C₃-C₆)cycloalkyl or phenyl groups being optionally substituted with one or two halogen atom(s) or (C₁-C₃)alkyl group(s);
      a (C₃-C₆)cycloalkyl(C₁-C₃)alkyl group, optionally substituted on the cycloalkyl with 1 to 4 substituents independently selected from: a fluorine atom, a —OH group, a (C₁-C₄)alkyl group, a (C₁-C₃)fluoroalkyl group, a (C₁-C₃)fluoroalkoxy group and an oxo group;
      a 4 to 7 membered-heterocycloalkyl group comprising 1 or 2 heteroatoms independently selected from oxygen, nitrogen and sulfur, such as a tetrahydropyranyl group, said heterocycloalkyl group being saturated or partially saturated and being optionally substituted with 1 to 3 substituents independently selected from: a fluorine atom, a (C₁-C₃)alkyl group, a (C₁-C₃)fluoroalkyl group, a (C₁-C₃)fluoroalkoxy group, an oxo group, a (C₁-C₃)alkoxy group, and a —OH group;
      a (C₁-C₆)alkyl group, such as an isobutyl group, a methyl group or an ethyl group, said alkyl group being optionally substituted with 1 to 4 substituents independently selected from: a fluorine atom, a (C₁-C₃)alkoxy group, a (C₁-C₃)fluoroalkoxy group and a —OH group; and
      a phenyl(C₁-C₂)alkyl group, said phenyl group being optionally substituted with 1 to 3 substituents independently selected from a halogen atom; a (C₁-C₃)alkyl group; a (C₁-C₃)fluoroalkyl group; a (C₁-C₃)alkoxy group; a (C₁-C₃)fluoroalkoxy group; a cyano group; and a —OH group;
X represents —CH₂—, —O— or —S—;
Y represents —CH═, —N═ or —CR″═, wherein R″ represents a (C₁-C₃)alkyl group, a halogen atom, such as a fluorine or a chlorine atom, a cyano group, or a (C₁-C₃)fluoroalkyl group, such as trifluoromethyl;
R7 independently represents a (C₁-C₃)alkyl group such as methyl, a halogen atom such as a fluorine atom, a cyano group, or a (C₁-C₃)fluoroalkyl group such as trifluoromethyl;
R8 represents a hydrogen atom or a fluorine atom;
R9 represents a hydrogen atom, a (C₁-C₃)alkyl group or a cyclopropyl;
n is 0, 1 or 2; and
m is 0 or 1.

The compounds of formula (I) can contain one or more asymmetric carbon atoms. They may therefore exist in the form of enantiomers.

The compounds of formula (I) may be present as well under tautomer forms.

The compounds of formula (I) may exist in the form of bases, acids, zwitterion or of addition salts with acids or bases. Hence, herein are provided compounds of formula (I) or pharmaceutically acceptable salts thereof.

These salts may be prepared with pharmaceutically acceptable acids or bases, although the salts of other acids or bases useful, for example, for purifying or isolating the compounds of formula (I) are also provided.

Among suitable salts of the compounds of formula (I), hydrochloride may be cited As used herein, the terms below have the following definitions unless otherwise mentioned throughout the instant specification:
a halogen atom: a fluorine, a chlorine, a bromine or an iodine atom, and in particular a fluorine and a chlorine atom;
an oxo: a “═O” group;
an alkyl group: a linear or branched saturated hydrocarbon-based aliphatic group comprising, unless otherwise mentioned, from 1 to 6 carbon atoms (noted “(C₁-C₆)alkyl”). By way of examples, mention may be made of, but not limited to: methyl, ethyl, propyl, isopropyl, butyl, isobutyl, sec-butyl, tert-butyl, pentyl, isopentyl, hexyl and isohexyl groups, and the like;
a cycloalkyl group: a monocyclic alkyl group comprising, unless otherwise mentioned, from 3 to 7 carbon atoms, saturated or partially unsaturated and unsubstituted or substituted. By way of examples, mention may be made of, but not limited to: cyclopropyl, cyclobutyl, cyclopentyl, cyclobutenyl, cyclopentenyl, cyclohexyl, cyclohexenyl, cycloheptyl and cycloheptenyl groups and the like, in particular a cyclopentyl, a cyclohexyl or a cyclohexenyl;
a cycloalkylalkyl group: an alkyl group substituted with a cyclic alkyl group as defined above. Mention may be made of, but not limited to: cyclobutylmethyl;
a heterocycloalkyl group: a 4 to 7-membered cycloalkyl group, saturated or partially unsaturated, comprising 1 to 2 heteroatoms independently selected from oxygen, nitrogen and sulfur, in particular being oxygen or nitrogen. By way of examples, mention may be made of, but not limited to: morpholinyl, piperazinyl, piperidinyl, pyrrolidinyl, aziridinyl, oxanyl, oxetanyl, tetrahydropyranyl, morpholinyl, tetrahydrofuranyl, oxepanyl, diazepanyl, dioxanyl, tetrahydropyranyl and tetrahydrothiopyranyl. The heterocycloalkyl is advantageously tetrahydropyranyl.
a fluoroalkyl group: an alkyl group as previously defined where the alkyl group is substituted with at least one fluorine atom. In other terms, at least one hydrogen atom of the alkyl group is replaced by a fluorine atom. By way of example, mention may be made of —CH₂F, —CHF₂, —CH₂CHF₂, —CH₂CH₂F and the like. When all the hydrogen atoms belonging to the alkyl group are replaced by fluorine atoms, the fluoroalkyl group can be named perfluoroalkyl group. By way of example, mention may be made of trifluoromethyl group or trifluoroethyl group and the like, and in particular trifluoromethyl group;
an alkoxy group: an —O-alkyl group where the alkyl group is as previously defined. By way of examples, mention may be made of, but not limited to: methoxy, ethoxy, propoxy, isopropoxy, linear, secondary or tertiary butoxy, isobutoxy, pentoxy or hexoxy groups, and the like;
a fluoroalkoxy group: an —O-alkyl group where the alkyl group is as previously defined and where the alkyl group is substituted with at least one fluorine atom. In other terms, at least one hydrogen atom of the alkyl group is replaced by a fluorine atom. By way of example, mention may be made of —OCH₂F, —OCHF₂, —OCH₂CH₂F and the like. When all the hydrogen atoms belonging to the alkyl group are replaced by fluorine atoms, the fluoroalkoxy group can be named perfluoroalkoxy group. By way of example, mention may be made of trifluoromethoxy group and the like;
a (C₁-C₄)alkylthio group also named (C₁-C₄)alkylsulfanyl: a —S-alkyl group where the alkyl group is as previously defined. By way of examples, mention may be made of, but not limited to: methylthio, ethylthio, propylthio, isopropylthio, linear, secondary or tertiary butylthio, isobutylthio, and the like;
a (C₁-C₄)alkylsulfonyl group: a —SO₂-alkyl group where the alkyl group is as previously defined. By way of examples, mention may be made of, but not limited to: —SO₂CH₃, —SO₂CH₂CH₃ and the like;
a (C₁-C₄)fluoroalkylthio group also named a (C₁-C₄)fluoroalkylsulfanyl group: a —S-fluoroalkyl group where the fluoroalkyl group is as previously defined. By way of examples, mention may be made of, but not limited to: fluoromethylthio, difluoromethylthio, trifluoromethylthio and the like;
a fused phenyl: a bicyclic radical comprising from 7 to 10 carbon atoms and that contains a phenyl moiety. Said phenyl moiety may be fused to a (C₃-C₆)cycloalkyl group, i.e. the phenyl moiety may share a bond with said (C₃-C₆)cycloalkyl group. The fused phenyl group may be bound to the rest of the molecule by its phenyl moiety. It may be substituted. Examples are, but not limited to, indanyl, bicyclo[4.2.0]octa-1(6),2,4-trienyl, tetrahydronaphthalenyl and the like;
a heteroaryl group: a 5 to 10-membered cyclic aromatic group containing between 2 and 9 carbon atoms and containing between 1 and 3 heteroatoms, such as nitrogen, oxygen or sulfur. Such nitrogen atom may be substituted with an oxygen atom in order to form a —N—O bond. Such —N—O bond can be in a form of a N-oxide (—N⁺—O⁻). Said heteroaryl group may be monocyclic or bicyclic. By way of examples of heteroaryl groups, mention may be made of, but not limited to: thiophene, furan, thiadiazole, thiazole, imidazole, pyridazine, triazine, pyrazine, oxadiazole, pyrazole, isothiazole, oxazole, isoxazole, pyridine, pyrimidine, benzotriazole, benzoxazole, pyrrolo[2,3-b]pyridine, benzimidazole, benzoxadiazole, benzothiazole, benzothiadiazole, benzofuran, indole, isoquinoline, indazole, benzisoxazole, benzisothiazole, pyridone groups and the like. The heteroaryl is advantageously pyridine, pyrrole, imidazole, pyrazine, furane, thiazole, pyrazole, thiadiazole, pyridazine, pyridone and pyrimidine, and more particularly pyridine;
a bicyclic group, generally comprising 5 to 12 carbon atoms, is a hydrocarbon group selected from groups comprising two rings connected through:
   a single common atom: a “spirobicyclic ring”. Such spiro bicyclic alkyl generally comprises 5 to 11 carbon atoms referring to a “spiro(C₅-C₁₁)bicyclic ring”. The rings may be saturated or partially unsaturated. Such spirobicyclic ring may be unsubstituted or substituted, in particular by at least one (C₁-C₃)alkyl group such as methyl or a fluorine. By way of examples of spiro(C₅-C₁₁)bicyclic ring as for the definition of R6, mention may be made of, but not limited to: spiro[2.3]hexane, spiro[3.3]heptane, spiro[3.3]heptene, spiro[2.5]octane and 7-azaspiro[3.5]nonane. The spiro(C₅-C₁₁)bicyclic ring is advantageously spiro[2.3]hexane, spiro[3.3]heptane or spiro[3.3]heptene still for the R6 group;
   two common atoms: In that case the bicyclic group comprises 7 to 12 carbon atoms and optionally comprises 1 to 2 unsaturations. By way of examples of such bicyclic groups, mention may be made of, but not limited to: cis-1,3a,4,5,6,6a-hexahydropentalenyl group, bicyclo[3.1.0]hexan-1-yl, bicyclo[4.1.0]heptanyl and octahydropentalenyl,
   three or more common atoms: In that case the bicyclic group comprises 6 to 10 carbon atoms, such bicyclic group may be referred to as a “bridged (C₆-C₁₀)cycloalkyl” group, the rings share three or more atoms and the bridge contains at least one atom, for example 1, 2 or 3 atoms and preferentially 1 atom. By way of examples of such bridged cycloalkyl groups, mention may be made of, but not limited to bicyclo[3.2.1]octan-3-yl and bicyclo[2.2.1]heptan-2-yl.
a zwitterion means: a globally neutral molecule with a positive and a negative electrical charge and having an acidic group and a basic group.

In another embodiment, in the compounds of formula (I) as defined above, R1 and R2 are a hydrogen atom.

In another embodiment, in the compounds of formula (I) as defined above, R3 is a —COOH group.

In another embodiment, in the compounds of formula (I) as defined above, R3′ and R3″ both represent a hydrogen atom.

In another embodiment, in the compounds of formula (I) as defined above, X represents —CH₂—.

In another embodiment, in the compounds of formula (I) as defined above, R4 and R4′ both represent a hydrogen atom or one of R4 and R4′ represents a hydrogen atom and the other a fluorine atom.

In another embodiment, in the compounds of formula (I) as defined above, R5 represents a hydrogen atom.

In another embodiment, in the compounds of formula (I) as defined above, R6 represents a phenyl group, said phenyl group being optionally substituted with 1 to 3 substituents independently selected from a halogen atom; a (C₁-C₆)alkyl group and a (C₁-C₆)fluoroalkyl group, more particularly selected from a chlorine atom, a fluorine atom, a methyl group and a trifluoromethyl group. In such embodiment, in particular, at least one of the substituents of said phenyl group comprises a halogen atom.

In another embodiment, in the compounds of formula (I) as defined above, n is 0 or 1 and R7 is a fluorine atom.

In another embodiment, in the compounds of formula (I) as defined above, n is 0.

In another embodiment, in the compounds of formula (I) as defined above, Y represents —CH═.

In another embodiment, in the compounds of formula (I) as defined above, m is 1.

In another embodiment, in the compounds of formula (I) as defined above, m is 0.

In another embodiment, in the compounds of formula (I) as defined above, R3 is a —COOH group and R6 is a phenyl group comprising one, two or three substitutions, more particularly two substitutions, independently selected from a chlorine atom, a fluorine atom, a trifluoromethyl group and a methyl group, at least one of the substitutions comprising a halogen atom. In such embodiment, R3′ and R3″ are in particular hydrogen atoms. Still in such embodiment, R1, R2, R5, R8 and R9 are hydrogen atoms. In such embodiment, both R4 and R4′ are hydrogen atoms or one of R4 and R4′ is a hydrogen atom and the other is a fluorine atom. Still in such embodiment, Y is a —CH═ group, m is equal to 1 and n is equal to 0. Still in such embodiment, X is a —CH₂— group.

Among the compounds of formula (I) described herein, mention may be made in particular of the following compounds or a pharmaceutically acceptable salt thereof, in particular hydrochloride salt thereof:
(Z)-8-(2,4-dichlorophenyl)-9-(4-((1-(3,3-fluoropropyl)pyrrolidin-3-ylidene)methyl)phenyl)-6,7-dihydro-5H-benzo[7]annulene-3-carboxylic acid hydrochloride (1),
(Z)-8-(2-chloro-4-fluorophenyl)-9-(4-((1-(3,3-fluoropropyl)pyrrolidin-3-ylidene)methyl)phenyl)-6,7-dihydro-5H-benzo[7]annulene-3-carboxylic acid (2),
(Z)-4-(2,4-dichlorophenyl)-5-(4-((1-(3-fluoropropyl)pyrrolidin-3-ylidene)methyl)phenyl)-2,3-dihydrobenzo[b]oxepine-8-carboxylic acid hydrochloride (3),
Z)-8-(2,4-dichlorophenyl)-9-(4-((1-(3,3-difluoropropyl)pyrrolidin-3-ylidene)methyl)phenyl)-6,7-dihydro-5H-benzo[7]annulene-3-carboxylic acid (4),
(Z)-8-(2,4-difluorophenyl)-9-(4-((1-(3,3-difluoropropyl)pyrrolidin-3-ylidene)methyl)phenyl)-6,7-dihydro-5H-benzo[7]annulene-3-carboxylic acid hydrochloride (5),
(Z)-8-(4-fluoro-2-methylphenyl)-9-(4-((1-(3-fluoropropyl)pyrrolidin-3-ylidene)methyl)phenyl)-6,7-dihydro-5H-benzo[7]annulene-3-carboxylic acid (6),
(Z)-3-(4-(8-(2-chlorophenyl)-6,7-dihydro-5H-benzo[7]annulen-9-yl)benzylidene)-1-(3-fluoropropyl)pyrrolidine (7),
(Z)-8-(4-fluoro-2-(trifluoromethyl)phenyl)-9-(4-((1-(3-fluoropropyl)pyrrolidin-3-ylidene)methyl)phenyl)-6,7-dihydro-5H-benzo[7]annulene-3-carboxylic acid (8),
(Z)-8-(2,4-difluorophenyl)-9-(4-((1-(3,3-fluoropropyl)pyrrolidin-3-ylidene)methyl)phenyl)-6,7-dihydro-5H-benzo[7]annulene-3-carboxylic (9),
(Z)-8-(3-chloro-2-methylphenyl)-9-(4-((1-(3-fluoropropyl)pyrrolidin-3-ylidene)methyl)phenyl)-6,7-dihydro-5H-benzo[7]annulene-3-carboxylic acid (10),
(Z)-9-(4-((1-(3-fluoropropyl)pyrrolidin-3-ylidene)methyl)phenyl)-8-(3-methyl-2-(trifluoromethyl)phenyl)-6,7-dihydro-5H-benzo[7]annulene-3-carboxylic acid (11),
(Z)-9-(4-((1-(3,3-difluoropropyl)pyrrolidin-3-ylidene)methyl)phenyl)-8-(3-methyl-2-(trifluoromethyl)phenyl)-6,7-dihydro-5H-benzo[7]annulene-3-carboxylic acid (12),
(Z)-8-(3-chloro-2-methylphenyl)-9-(4-((1-(3,3-difluoropropyl)pyrrolidin-3-ylidene)methyl)phenyl)-6,7-dihydro-5H-benzo[7]annulene-3-carboxylic acid (13),
(Z)-9-(4-((1-(3-fluoropropyl)pyrrolidin-3-ylidene)methyl)phenyl)-8-(2-methyl-3-(trifluoromethyl)phenyl)-6,7-dihydro-5H-benzo[7]annulene-3-carboxylic acid (14),
(Z)-9-(4-((1-(3,3-difluoropropyl)pyrrolidin-3-ylidene)methyl)phenyl)-8-(4-fluoro-2-methylphenyl)-6,7-dihydro-5H-benzo[7]annulene-3-carboxylic acid (15),
(Z)-8-(2,4-dichlorophenyl)-9-(4-((1-(3-fluoropropyl)pyrrolidin-3-ylidene)methyl)phenyl)-6,7-dihydro-5H-benzo[7]annulen-3-ol hydrochloride (16),
(Z)-8-(2,4-dichlorophenyl)-9-(4-((1-(3-difluoropropyl)pyrrolidin-3-ylidene)methyl)phenyl)-6,7-dihydro-5H-benzo[7]annulen-3-ol hydrochloride (17),
(Z)-9-(4-((1-(3,3-difluoropropyl)pyrrolidin-3-ylidene)methyl)phenyl)-8-(4-fluoro-2-(trifluoromethyl)phenyl)-6,7-dihydro-5H-benzo[7]annulene-3-carboxylic acid (18),
(Z)-9-(4-((1-(3,3-difluoropropyl)pyrrolidin-3-ylidene)methyl)phenyl)-8-(2-methyl-3-(trifluoromethyl)phenyl)-6,7-dihydro-5H-benzo[7]annulene-3-carboxylic acid (19),
(Z)-9-(4-((1-(3,3-difluoropropyl)pyrrolidin-3-ylidene)methyl)phenyl)-8-(4-fluoro-2,6-dimethyl)phenyl)-6,7-dihydro-5H-benzo[7]annulene-3-carboxylic acid (20),
(Z)-8-(4-fluoro-2,6-dimethylphenyl)-9-(4-((1-(3-fluoropropyl)pyrrolidin-3-ylidene)methyl)phenyl)-6,7-dihydro-5H-benzo[7]annulene-3-carboxylic acid (21),
(Z)-9-(4-((1-(3-fluoropropyl)pyrrolidin-3-ylidene)methyl)phenyl)-8-(2-(trifluoromethyl)phenyl)-6,7-dihydro-5H-benzo[7]annulene-3-carboxylic acid (22),
(Z)-8-(2,4-dichlorophenyl)-9-(3-fluoro-4-((1-(3-fluoropropyl)pyrrolidin-3-ylidene)methyl)phenyl)-6,7-dihydro-5H-benzo[7]annulene-3-carboxylic acid (23),
(Z)-6-(2,4-dichlorophenyl)-5-(4-((1-(3-fluoropropyl)pyrrolidin-3-ylidene)methyl)phenyl)-7,8-dihydronaphthalene-2-carboxylic acid hydrochloride (24),
(Z)-9-(4-((1-(3-fluoropropyl)pyrrolidin-3-ylidene)methyl)phenyl)-8-(3-(trifluoromethyl)phenyl)-6,7-dihydro-5H-benzo[7]annulene-3-carboxylic acid (25),
(Z)-9-(4-((1-(3-fluoropropyl)pyrrolidin-3-ylidene)methyl)phenyl)-8-(4-(trifluoromethyl)phenyl)-6,7-dihydro-5H-benzo[7]annulene-3-carboxylic acid (26),
(Z)-8-(3-chlorophenyl)-9-(4-((1-(3-fluoropropyl)pyrrolidin-3-ylidene)methyl)phenyl)-6,7-dihydro-5H-benzo[7]annulene-3-carboxylic acid (27),
(Z)-8-(4-chlorophenyl)-9-(4-((1-(3-fluoropropyl)pyrrolidin-3-ylidene)methyl)phenyl)-6,7-dihydro-5H-benzo[7]annulene-3-carboxylic acid (28),
(Z)-8-(4-fluoro-2,5-dimethylphenyl)-9-(4-((1-(3-fluoropropyl)pyrrolidin-3-ylidene)methyl)phenyl)-6,7-dihydro-5H-benzo[7]annulene-3-carboxylic acid (29),
(Z)-4-(2,4-dichlorophenyl)-5-(4-((1-(3-fluoropropyl)pyrrolidin-3-ylidene)methyl)phenyl)-2,3-dihydrobenzo[b]thiepin-8-ol (30).

Another embodiment is a compound selected from the above list, or a pharmaceutically acceptable salt thereof, for use in therapy, especially as an inhibitor and degrader of estrogen receptors.

Another embodiment is a compound selected from the above list, or a pharmaceutically acceptable salt thereof, for use in the treatment of cancer, especially breast cancer.

Another embodiment is a method of inhibiting and degrading estrogen receptors, comprising administering to a subject in need thereof, in particular a human, a therapeutically effective amount of a compound selected from the above list, or a pharmaceutically acceptable salt thereof.

Another embodiment is a method of treating ovulatory dysfunction, cancer, endometriosis, osteoporosis, benign prostatic hypertrophy or inflammation, comprising administering to a subject in need thereof, in particular a human, a therapeutically effective amount of a compound selected from the above list, or a pharmaceutically acceptable salt thereof.

Another embodiment is a method of treating cancer, comprising administering to a subject in need thereof, in particular a human, a therapeutically effective amount of a compound selected from the above list, or a pharmaceutically acceptable salt thereof.

Another embodiment is a pharmaceutical composition comprising as active principle an effective dose of a compound selected from the above list, or a pharmaceutically acceptable salt thereof, and also at least one pharmaceutically acceptable excipient.

The compounds of the formula (I) can be prepared by the following processes.

The compounds of the formula (I) and other related compounds having different substituents are synthesized using techniques and materials described below or otherwise known by the skilled person in the art. In addition, solvents, temperatures and other reaction conditions presented below may vary as deemed appropriate to the skilled person in the art.

General below methods for the preparation of compounds of formula (I) optionally modified by the use of appropriate reagents and conditions for the introduction of the various moieties found in the formula (I) as described below.

The following abbreviations and empirical formulae are used:
MeCN Acetonitrile
NH₄Cl Ammonium chloride
CO Carbon monoxide
CO₂ Carbon dioxide
Cs₂CO₃ Cesium carbonate
DBU 1,8-Diazabicyclo[5.4.0]undec-7-ene
DCM Dichloromethane
Et₂O Diethyl ether
DIEA Diisopropylethylamine
DMF N,N-dimethylformamide
DMAP Dimethylaminopyridine
DMSO Dimethyl sulfoxide
BOC₂O di-ter-butyldicarbonate
EtOH Ethanol
EtOAc Ethyl acetate
HCl Hydrochloric acid
LiHMDS Lithium bis(trimethylsilyl)amide
LiOH Lithium hydroxide
MeOH Methanol
MgSO₄ Magnesium sulfate
MTBE Methyl tert-butyl ether
MeTHF 2-Methyltetrahydrofuran
n-BuLi n-Butyllithium
KOAc Potassium acetate
KHMDS Potassium bis(trimethylsilyl)amide
K₂CO₃ Potassium carbonate
KOH Potassium hydroxide
NaBH₄ Sodium borohydride
NaHCO₃Sodium bicarbonate
NaH Sodium hydride
NaOH Sodium hydroxide
Na₂SO₄ Sodium sulfate
SCX Strong cation exchange
Pd(dppf)Cl₂ [1,1′-Bis(diphenylphosphino)ferrocene]dichloropalladium(II)
Pd(PPh₃)₂Cl₂ bis(triphenylphosphine) palladium(II) dichloride
Pd₂(dba)₃ tris(dibenzylideneacetone)dipalladium(0)
PhOK Potassium phenolate
SFC Supercritical Fluid Chromatography
TEA Triethylamine
TFA Trifluoroacetic acid
THF Tetrahydrofuran
PPh₃ Triphenylphosphine
RT Room temperature
XANTPHOS (9,9-dimethyl-9H-xanthene-4,5-diyl)bis(diphenylphosphane).

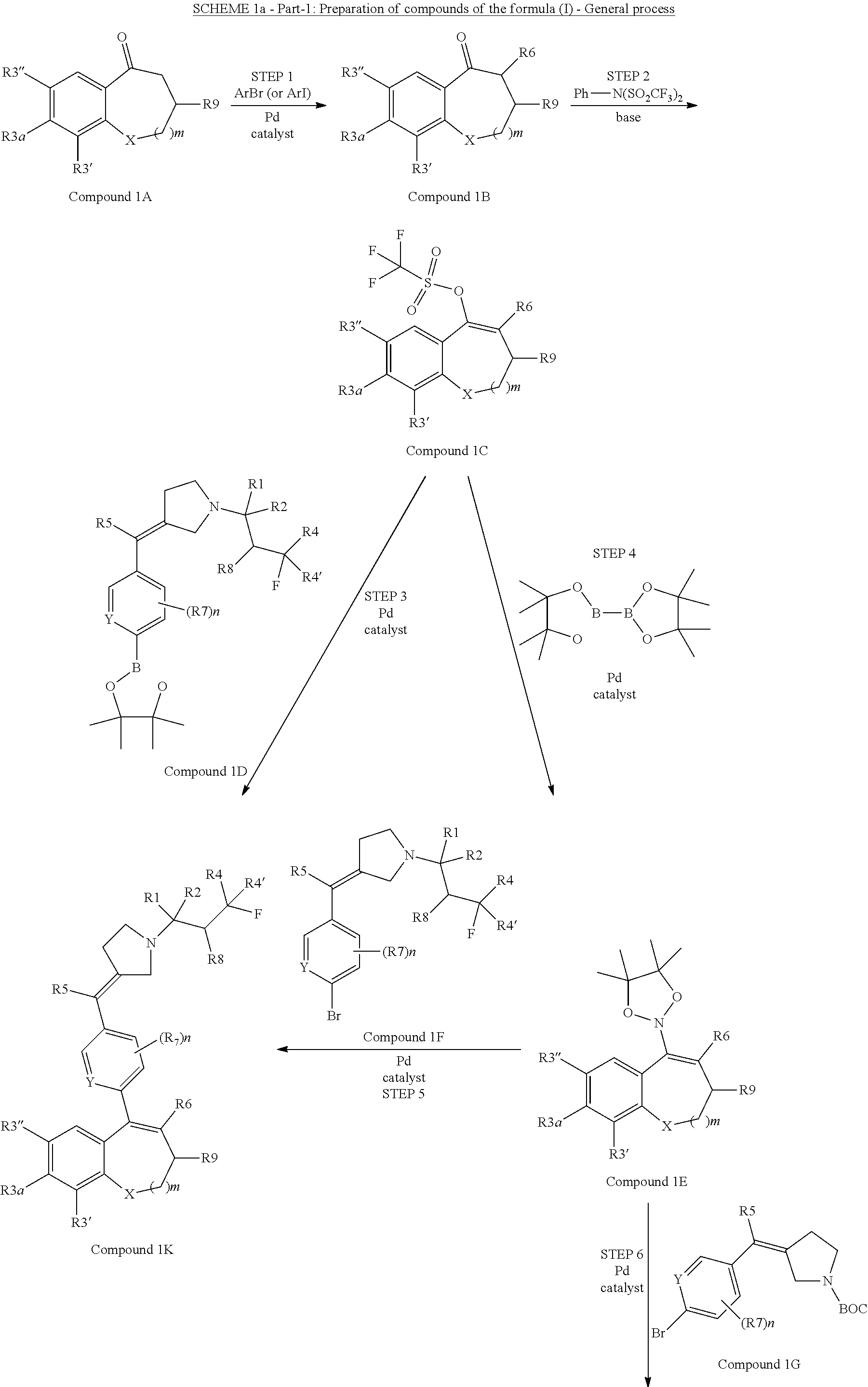

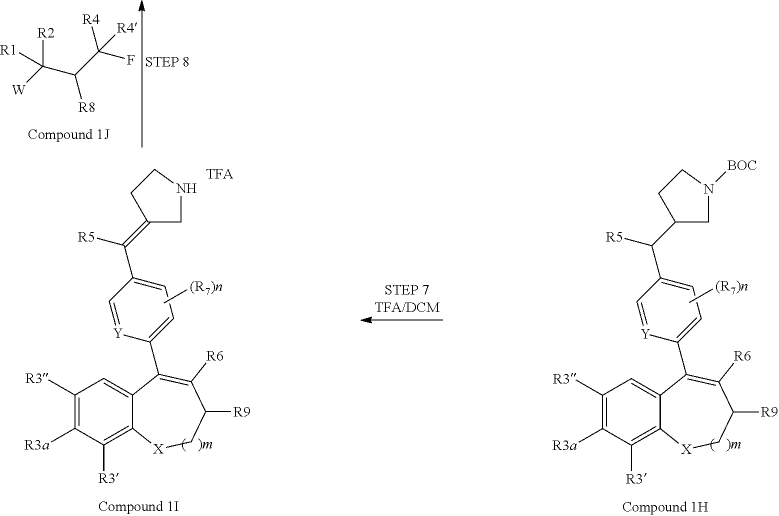

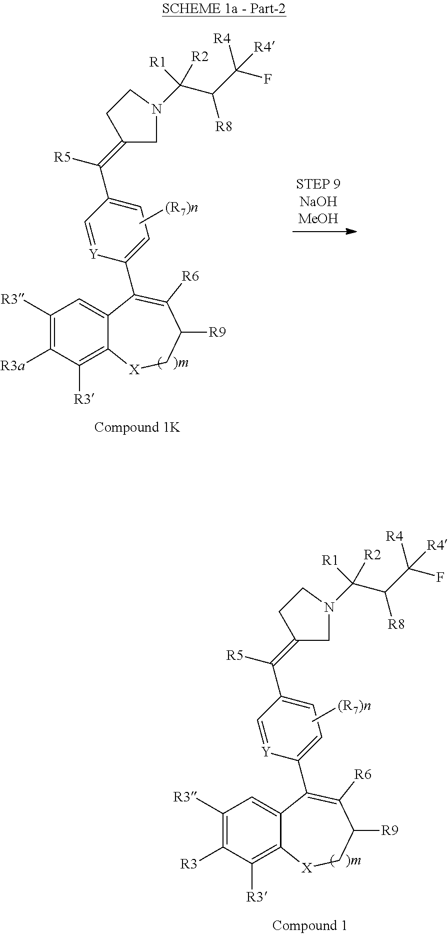

According to SCHEME 1a—Part-1 and Part-2, in which R3a is a hydrogen atom or a carboxylic ester such as COOMe, COOEt, or protected OH such as O-pivaloyl, R6 is a phenyl group or a heteroaryl group; and R1, R2, R3, R3′, R3″, R4, R4′, R5, R7, R8, R9 X, m, n and Y are as defined above, compound 1A can be converted in STEP 1 to compound 1B by treatment with aryl or heteroaryl bromide or iodide in the presence of a palladium catalyst, for example tris(dibenzylideneacetone)dipalladium(0) Pd₂(dba)₃, and a phosphine such as (9,9-dimethyl-9H-xanthene-4,5-diyl)bis(diphenylphosphane) (XANTPHOS) in solution in toluene or xylene by heating up to reflux of solvent, in presence of a base such as K₂CO₃ or Cs₂CO₃. Alternative way to prepare compound 1B, wherein R6 can be any of the groups defined above for R6 in formula (I), is described in SCHEME 1e below.

Compound 1B can be converted in STEP 2 to compound 1C by treatment with N,N-bis(trifluoromethylsulfonyl)aniline in the presence of a base such as DBU or NaH, or KHMDS at −50° C., in a solvent such as MeTHF.

Compound 1C can be converted in STEP 4 to compound 1E by treatment for example with bis(pinacolato)diboron, and with a palladium catalyst, for example bis (triphenylphosphine) palladium(II) dichloride Pd(PPh₃)₂Cl₂, and a phosphine, such as triphenylphosphine, in solution in toluene by heating up to reflux of solvent, in presence of a base such as KOPh.

Compound 1K can be prepared in a Suzuki coupling reaction either between compounds 1C and 1D in STEP 3 or between compounds 1E and 1F in STEP 5 using for example [1,1′-bis(diphenylphosphino)ferrocene]dichloropalladium(II) (Pd(dppf)Cl₂), complex with DCM, as catalyst, in a mixture of dioxane and water and in the presence of a base, for example cesium carbonate (Cs₂CO₃), by heating up to reflux of solvent.

Alternatively, compound 1E can be converted in STEP 6 to compound 1H in a Suzuki coupling reaction with compound 1G using for example [1,1′-bis(diphenylphosphino)ferrocene]dichloropalladium(II) (Pd(dppf)Cl₂), complex with DCM, as catalyst, in a mixture of dioxane and water and in the presence of a base, for example cesium carbonate (Cs₂CO₃), by heating up to reflux of solvent. Compound 1H can be converted in STEP 7 to compound 11 by treatment with TFA in solution in DCM or HCl in solution in dioxane. Compound 11 can be converted in STEP 8 to compound 1K by treatment with compound 1J, wherein W is Br, I or OSO₂R with R═CH₃, PhMe, CF₃ or CF₂CF₂CF₂CF₃, in presence of a base such as potassium carbonate in DMF at 70° C. or in presence of sodium hydroxide or potassium hydroxide in THF at room temperature or in presence of aqueous sodium hydroxide in DCM at room temperature.

When R3a is COOMe, COOEt, or a protected OH such as O-pivaloyl, compound 1K can be deprotected into compound I in STEP 9 by treating with an aqueous solution of sodium hydroxide (NaOH) or lithium hydroxide (LiOH), in MeOH. When R3 is COOH, extraction of compound could give the sodium salt of compound I. The acidification with an aqueous solution of HCl 2N to pH 6-7 could give the neutral form. The acidification with an aqueous solution of HCl 2N to pH 1-2 could give the hydrochloride salt. The purification using HPLC could give the formate or trifluoroacetate salt.

### SCHEME 1b—Part-1 and Part-2: Alternative Processes to Prepare Compounds of the Formula (I)—General Process

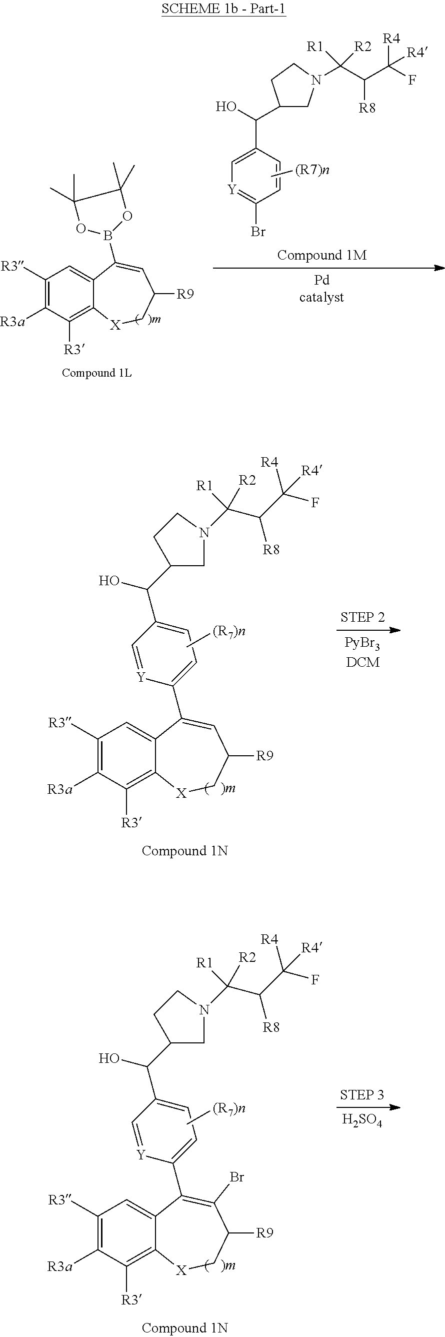

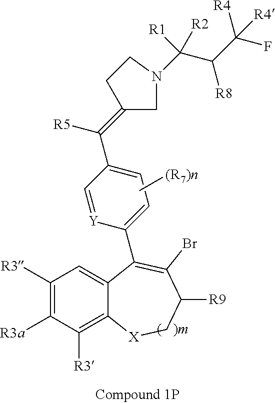

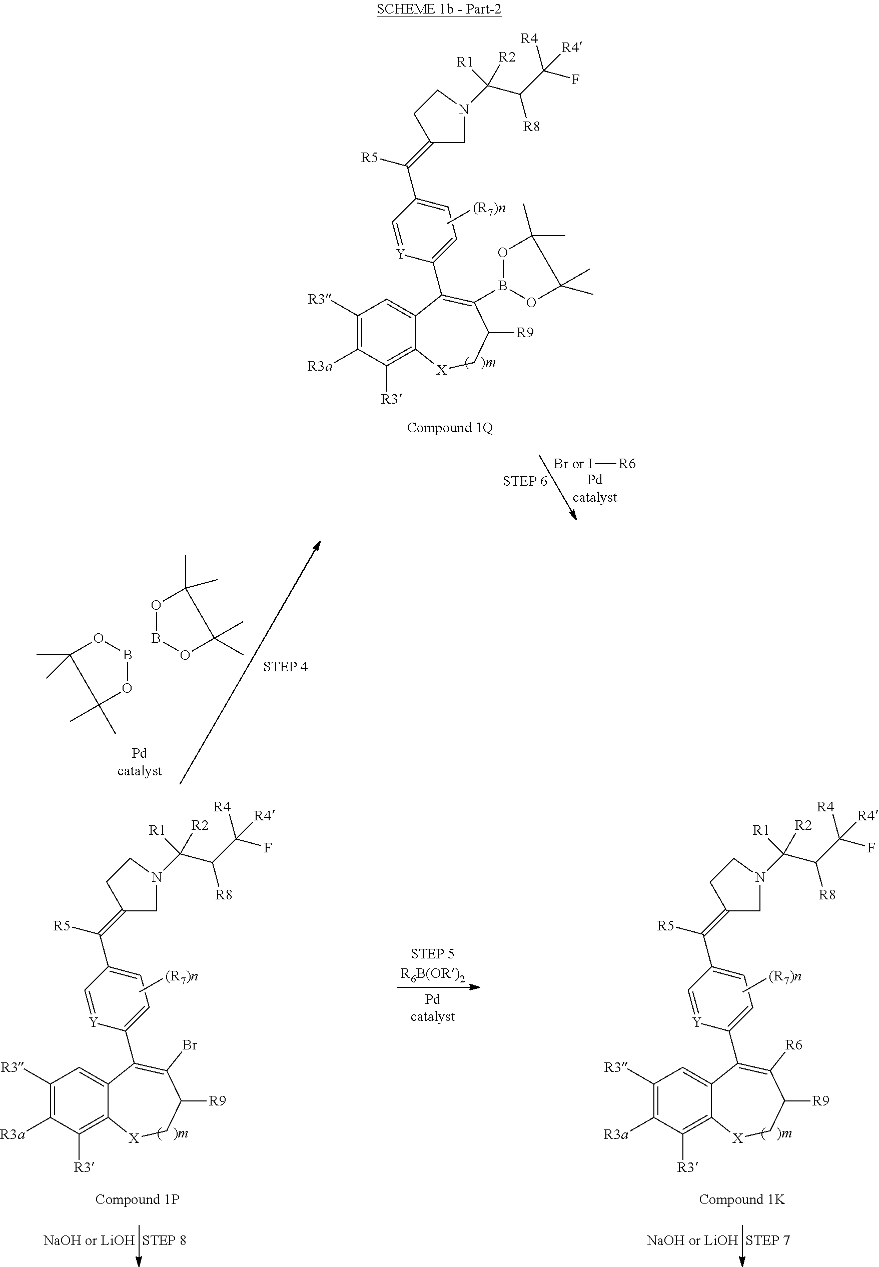

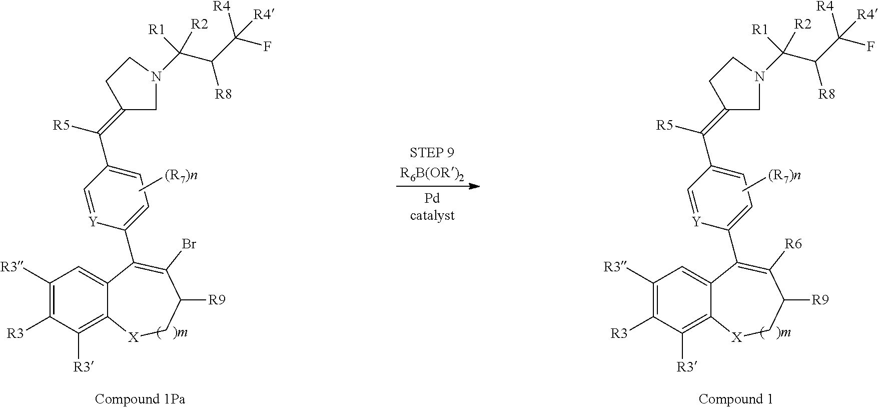

According to SCHEME 1b—Part-1 and —Part-2, in which R3a is a hydrogen atom, carboxylic ester such as COOMe, COOEt, or protected OH such as O-pivaloyl, R1, R2, R3, R3′, R3″, R4, R4′, R6, R7, R8, R9, X, n, m and Y are as defined above and R5 is a hydrogen atom, compound 1L can be converted in STEP 1 to compound 1N in a Suzuki coupling reaction with compound 1M using for example [1,1′-bis(diphenylphosphino)ferrocene]dichloropalladium(II) (Pd(dppf)Cl₂), complex with DCM, as catalyst, in a mixture of dioxane and water and in the presence of a base, for example Cs₂CO₃, by heating up to reflux of solvent.

Compound 1N can be converted in STEP 2 to compound 10 by treatment for example with pyridinium tribromide in DCM or THF at room temperature.

Compound 10 can be converted in STEP 3 to compound 1P by treatment with sulfuric acid in solution in water.

Compound 1P can be converted in STEP 4 to compound 1Q by treatment for example with bis(pinacolato)diboron, and with a palladium catalyst, for example bis (triphenylphosphine) palladium(II) dichloride Pd(PPh₃)₂Cl₂, and a phosphine such as triphenylphosphine in solution in toluene by heating up to reflux of solvent in presence of a base such as KOPh or AcOK.

Compound 1K can be prepared in a Suzuki coupling reaction either between compounds 1P and R6B(OR′)₂ or R6BF₃K, wherein —B(OR′)₂ is a boronic acid or a pinacolate ester and R6 is as defined above, in STEP 5 or between compounds 1Q and R6Br or R6I in STEP 6 using for example [1,1′-bis(diphenylphosphino)ferrocene]dichloropalladium(II) (Pd(dppf)Cl₂), complex with DCM, as catalyst, in a mixture of dioxane and water and in the presence of a base, for example cesium carbonate (Cs₂CO₃), by heating up to reflux of solvent.

Alternative way to prepare compound 1K, wherein R5 can be any of the groups defined above for R5 in formula (I), is described in SCHEME if below.

Compound 1K can be converted in STEP 7 to compound of formula (I) in presence of a source of hydroxide ions such as NaOH or LiOH in solution in MeOH.

Compound 1P can be converted in STEP 8 to compound 1 Pa in the presence of a source of hydroxide ions such as NaOH or LiOH in solution in MeOH.

This compound 1 Pa can be converted in STEP 9 to compound I through Suzuki conditions using a suitable boronic reagent R6B(OR′)₂ or R6BF₃K, wherein —B(OR′)₂ is a boronic acid or a pinacolate ester and R6 is as above defined, using for example Pd(dppf)Cl₂, complex with DCM, as catalyst, in a mixture of dioxane and water as solvent and in the presence of a base, for example Cs₂CO₃, at room temperature or by heating up to reflux of solvents.

When R3a is COOMe, COOEt, or a protected OH such as O-pivaloyl, deprotection can be performed in STEP 7 by treatment with an aqueous solution of NaOH 2N or LiOH in MeOH. When R3 is COOH, extraction of the product could give the sodium or lithium salt of compound I. The acidification with an aqueous solution of HCl 2N to pH 6-7 could give the neutral form of compound I. The acidification with an aqueous solution of HCl 2N to pH 1-2 could give the hydrochloride salt of compound I. The purification using HPLC in presence of formic acid or trifluoroacetic acid in the eluent could give the formate or trifluoroacetate salt of compound I.

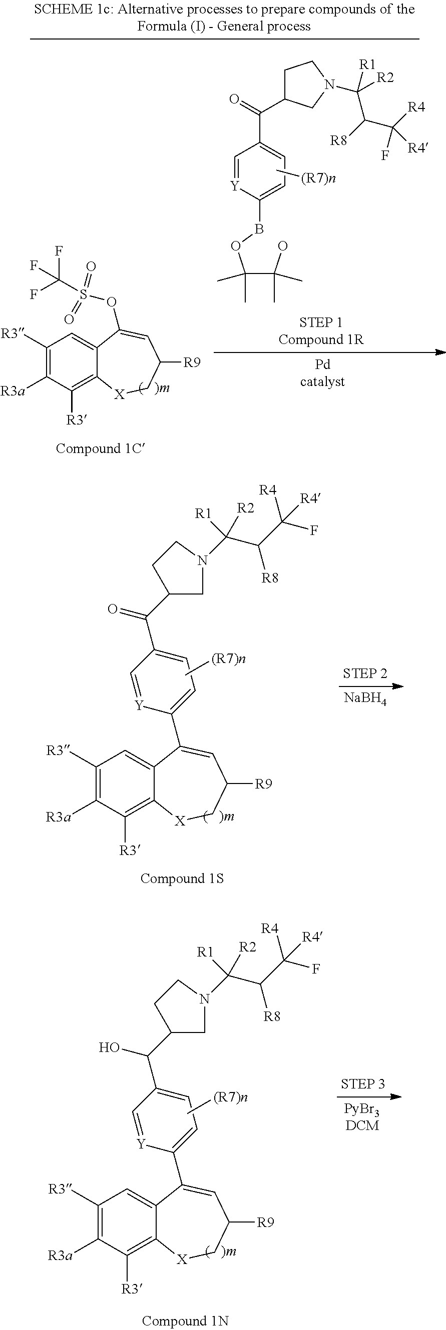

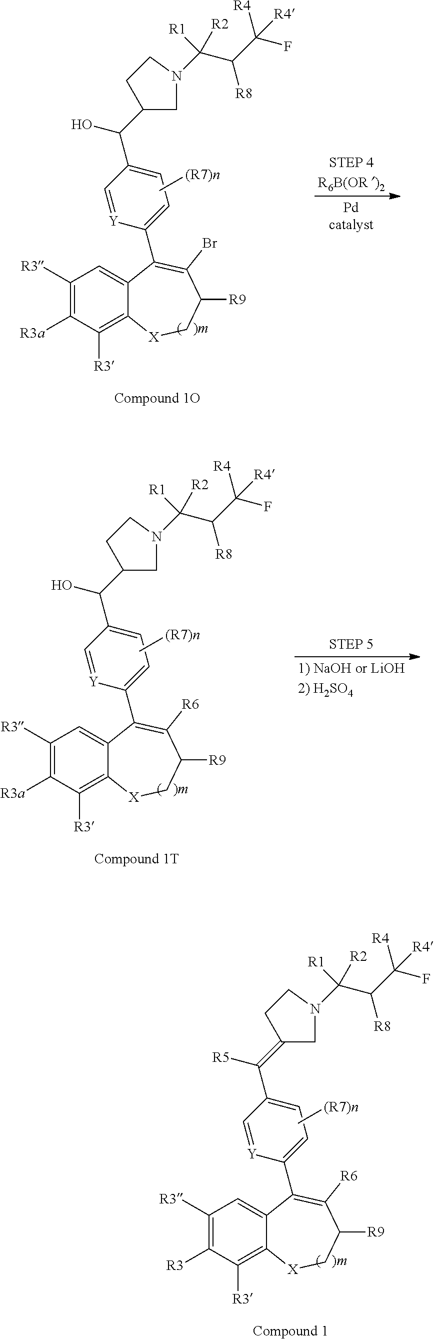

According to SCHEME 1c in which R3a is a hydrogen atom, carboxylic ester such as COOMe, COOEt, or protected OH such as O-pivaloyl, R1, R2, R3, R3′, R3″, R4, R4′, R6, R7, R8, R9, X, n, m and Y are as defined above, and R5 is a hydrogen atom, compound 1C′ can be converted in STEP 1 to compound 1S in a Suzuki coupling reaction with compound 1R using for example [1,1′-bis(diphenylphosphino) ferrocene]dichloropalladium(II) (Pd(dppf)C₁₂), complex with DCM, as catalyst, in a mixture of dioxane and water and in the presence of a base, for example Cs₂CO₃, by heating up to reflux of solvent.

Compound 1S can be converted in STEP 2 to compound 1N by treatment for example with NaBH₄ in a solvent such as MeOH.

Compound 1N can be converted in STEP 3 to compound 10 by treatment for example with pyridinium tribromide in DCM or THF at room temperature.

Compound 10 can be converted in STEP 4 to compound 1T through Suzuki conditions using a suitable boronic reagent R6B(OR′)₂ or R6BF₃K, wherein —B(OR′)₂ is a boronic acid or a pinacolate ester and R6 is as above defined, using for example [1,1′-bis(diphenylphosphino) ferrocene]dichloropalladium(II) (Pd(dppf)Cl₂), complex with DCM, as catalyst, in a mixture of dioxane and water and in the presence of a base, for example Cs₂CO₃, by heating up to reflux of solvent.

Compound 1T can be converted to compound I by treatment with a source of hydroxide ions such as NaOH or LiOH in solution in MeOH followed by a treatment with sulfuric acid.

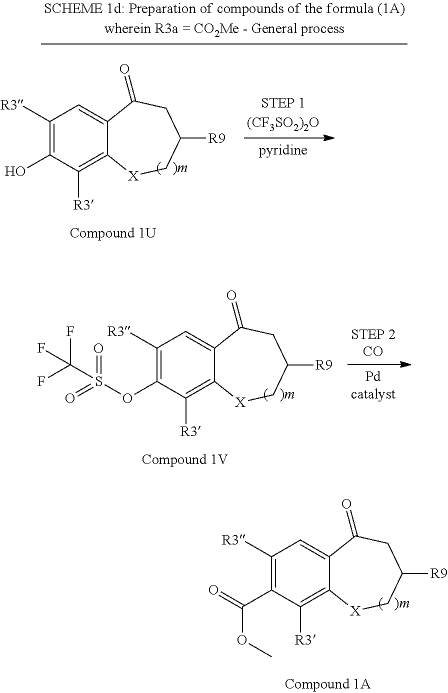

According to SCHEME 1d, in which X, m, R3′, R3″ and R9 are as defined above, compound 1A could be commercially available or prepared as follows: compound 1U (commercially available or prepared according to WO2017140669 and WO2018091153) can be converted in STEP 1 to compound 1V by treatment with trifluoromethanesulfonic anhydride, in solution in DCM, in the presence of pyridine as a base.

Compound 1V can be converted in STEP 2 to compound 1A by carbonylation with carbon monoxide, in solution in DMF and MeOH, in the presence of a palladium catalyst, for example [1,1′-bis(diphenylphosphino)ferrocene]dichloropalladium(II) (Pd(dppf)Cl₂), complex with DCM.

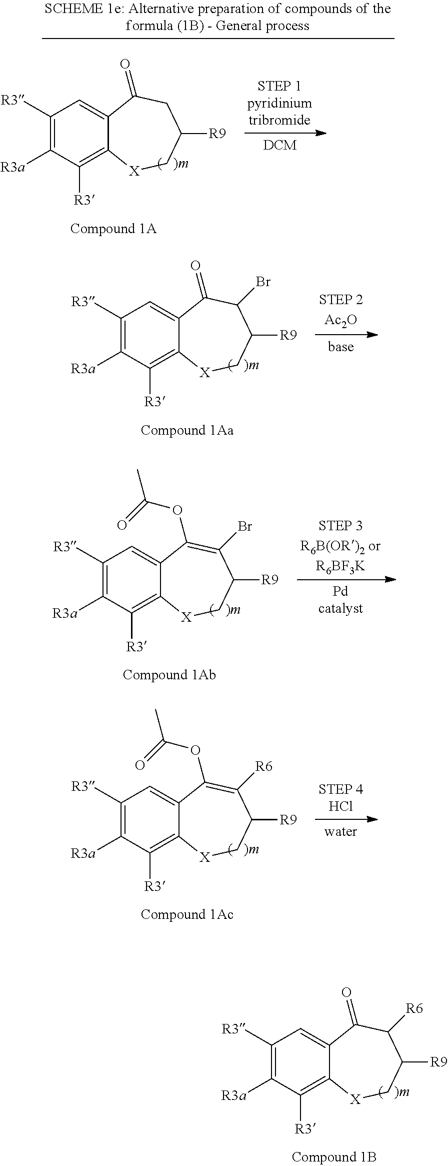

According to SCHEME 1e, in which R3a is a hydrogen atom, a carboxylic ester such as COOMe, COOEt, or protected OH such as O-pivaloyl, R3′, R3″, R9, R6, X and m are as defined above, compound 1B could alternatively be prepared as follows: compound 1A can be converted in STEP 1 to compound 1Aa by treatment with pyridinium tribromide in DCM or THF at room temperature for example.

Compound 1Aa can be converted in STEP 2 to compound 1Ab by deprotonation with a base such as LiHMDS in THF followed by treatment with acetic anhydride.

Compound 1Ac can be prepared in STEP 3 in a Suzuki coupling reaction between compounds 1Ab and R₆B(OR′)₂ or R₆BF₃K, wherein —B(OR′)₂ is a boronic acid or a pinacolate ester and R6 is as above defined, using for example [1,1′-bis(diphenylphosphino) ferrocene]dichloropalladium(II) (Pd(dppf)Cl₂), complex with DCM, as catalyst, in a mixture of toluene and water and in the presence of a base, for example cesium carbonate (Cs₂CO₃), by heating up to reflux of solvent.

Compound 1Ac can be converted in STEP 4 to compound 1B by hydrolysis with aqueous HCl solution by heating in methanol and DCM for example.

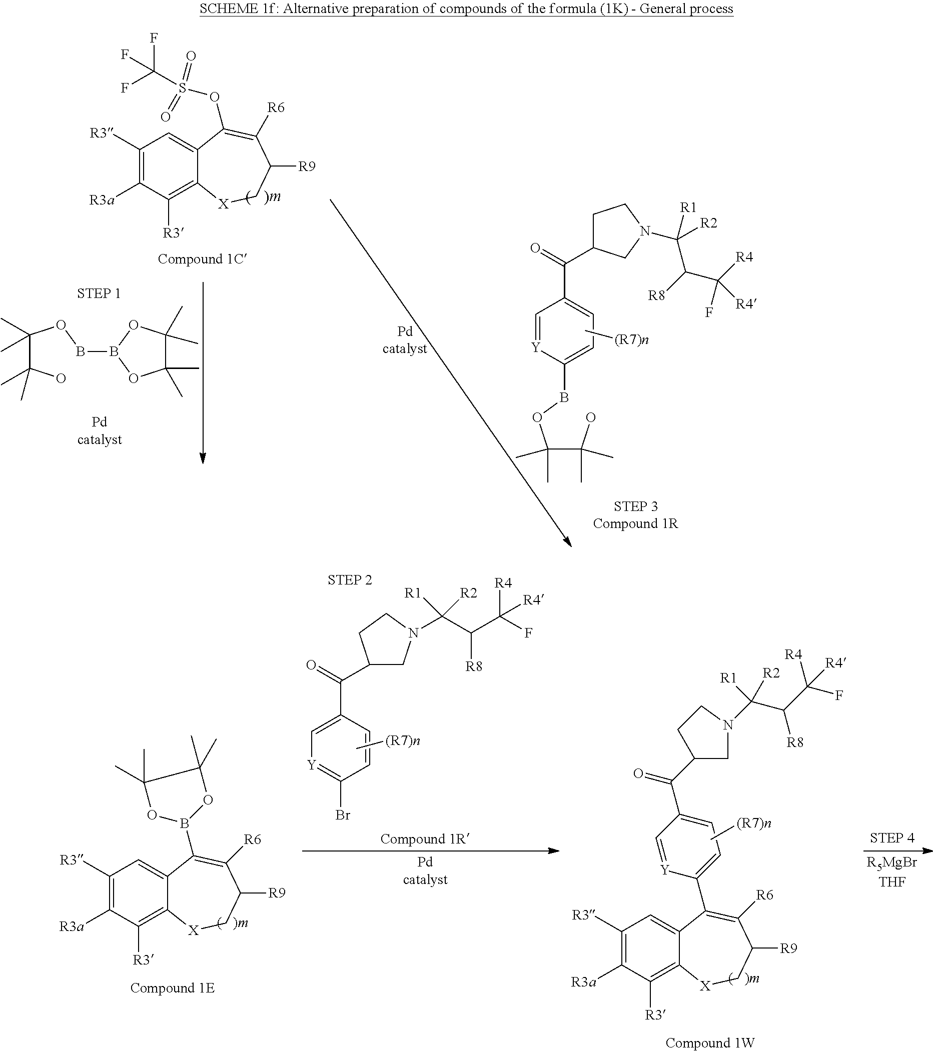

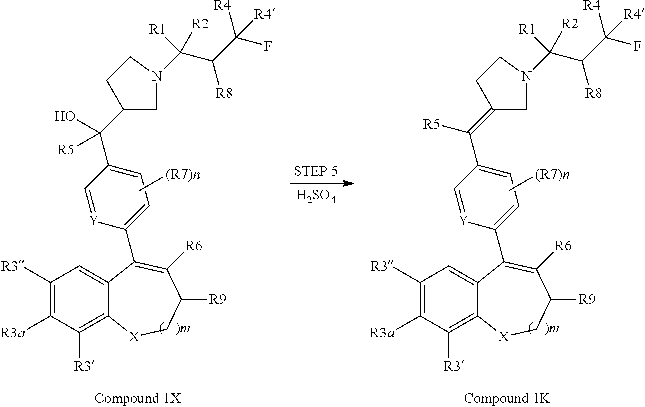

According to SCHEME 1f, in which R3a is a hydrogen atom or a carboxylic ester such as COOMe, COOEt, or protected OH such as O-pivaloyl, and R1, R2, R3, R3′, R3″, R4, R4′, R5, R6, R7, R8, R9 X, m, n and Y are as defined above, compound 1C can be converted in STEP 1 to compound 1E by treatment for example with bis(pinacolato)diboron, and with a palladium catalyst, for example bis(triphenylphosphine) palladium(II) dichloride Pd(PPh₃)₂Cl₂, and a phosphine, such as triphenylphosphine, in solution in toluene by heating up to reflux of solvent, in presence of a base such as KOPh.

Compound 1W can be prepared in a Suzuki coupling reaction either between compounds 1E and 1R′ in STEP 2 or between compounds 1C and 1R in STEP 3 using for example [1,1′-bis(diphenylphosphino)ferrocene]dichloropalladium(II) (Pd(dppf)Cl₂), complex with DCM, as catalyst, in a mixture of dioxane and water and in the presence of a base, for example cesium carbonate (Cs₂CO₃), by heating up to reflux of solvent.

Compound 1W can be converted in STEP 4 to compound 1X by treatment with alkyl magnesium bromide in a solvent such as THF.

Compound 1X can be converted in STEP 5 to compound 1K by treatment with sulfuric acid in water.

Herein is also provided a process for preparing a compound of formula (I) as defined above, wherein a compound of formula 1K

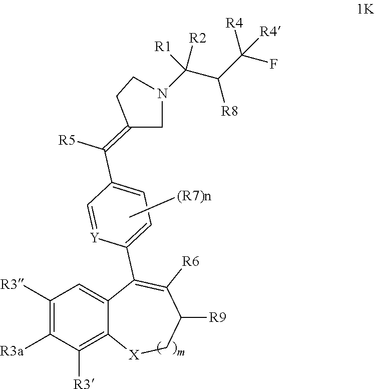

wherein R1, R2, R3′, R3″, R4, R4′, R5, R6, R7, R8, R9, m, n, X and Y are as defined above and R3a is a hydrogen atom, a carboxylic ester such as COOMe, COOEt, or a protected OH such as O-pivaloyl, is converted to compound of formula (I), in presence of a source of hydroxide ions, such as NaOH or LiOH in solution in methanol, said step being optionally preceded by a step for obtaining compound 1K, wherein a compound of formula 1P

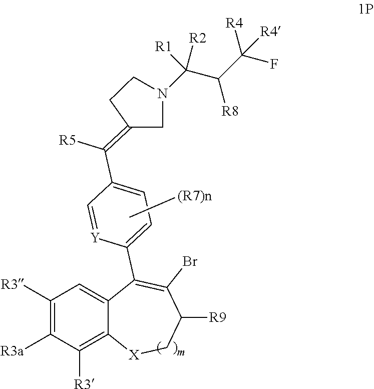

wherein R1, R2, R3′, R3″, R4, R4′, R5, R6, R7, R8, R9, m, n, X and Y are as defined above and R3a is as defined above,
is subjected to a Suzuki coupling with a boronic reagent R6-B(OR′)₂ or R6BF₃K, wherein —B(OR′)₂ is a boronic acid or a pinacolate ester and R6 is as defined above.

Herein is also provided a process for preparing a compound of formula (I) as defined above, wherein a compound of formula 1 Pa

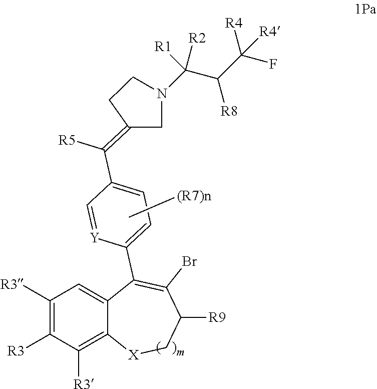

wherein R1, R2, R3a, R3′, R3″, R4, R4′, R5, R7, R8, R9, m, n, X and Y are as defined above, is submitted to a Suzuki coupling with a boronic reagent R6B(OR′)₂ or R6BF₃K, wherein —B(OR′)₂ is a boronic acid or a pinacolate ester and R6 is defined above, said step being optionally preceded by a step for obtaining compound 1 Pa, wherein a compound of formula 1P

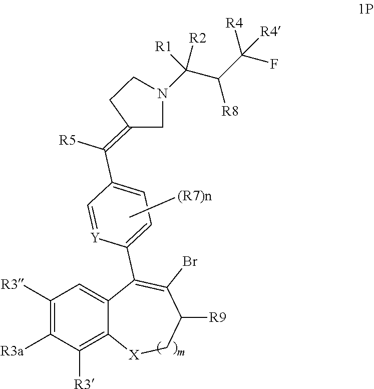

wherein R1, R2, R3′, R3″, R4, R4′, R5, R7, R8, R9, m, n, X and Y are as defined above and R3a is as defined above,
is converted to a compound 1 Pa in the presence of a source of hydroxide ions, such as NaOH or LiOH in solution in methanol.

Herein are also provided the intermediate compounds selected from compounds of formula 1P, 1K and 1 Pa, or any of their pharmaceutically acceptable salt,

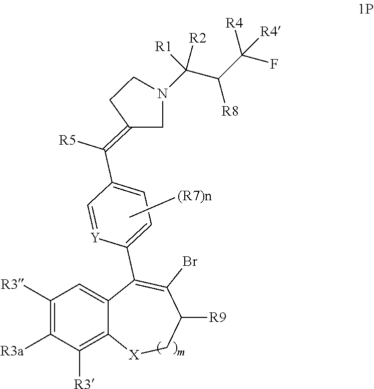

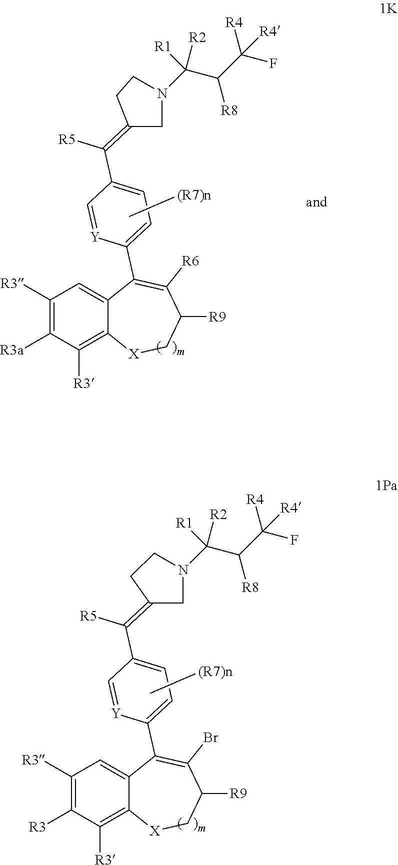

wherein R1, R2, R3′, R3″, R4, R4′, R5, R7, R8, R9, m, n, X and Y are as defined above and R3a is a hydrogen atom, a carboxylic ester such as COOMe, COOEt, or protected OH such as O-pivaloyl.

Herein is further provided the intermediate compounds of formula 1F and 1D, or any of its pharmaceutically acceptable salt

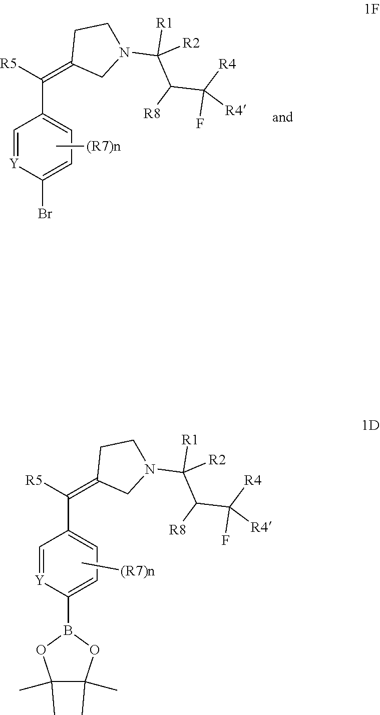

wherein R1, R2, R4, R4′, R5, R7, R8, Y and n are as defined above.

The present application also describes the intermediate compound of formula 1E, or any of its pharmaceutically acceptable salt

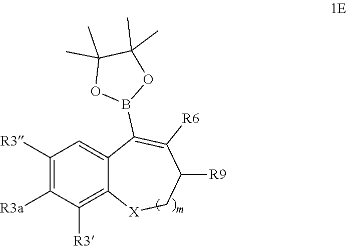

wherein R3a, R3′, R3″, X, m, R6 and R9 are as defined above.

In another aspect, herein is also provided a process for the preparation of a compound of formula (I), wherein R3 is a —COOH group, comprising a deprotection step of a compound of formula 1K as defined above, optionally followed by a purification step.

Said purification step may for example consist, as illustrated in step 2 of example 1 herein after, in an acidification step, for example with an aqueous solution of hydrochloric acid.

The ¹H NMR Spectra at 400 and 500 MHz were performed on a Bruker Avance DRX-400 and Bruker Avance DPX-500 spectrometer, respectively, with the chemical shifts (6 in ppm) in the solvent dimethyl sulfoxide-d6 (d6-DMSO) referenced at 2.5 ppm at a temperature of 303 K. Coupling constants (J) are given in Hertz.

The liquid chromatography/mass spectra (LC/MS) were obtained on a UPLC Acquity Waters instrument, light scattering detector Sedere and SQD Waters mass spectrometer using UV detection DAD 210-400 nm and flash Acquity UPLC CSH C18 1.7 μm, dimension 2.1×30 mm, mobile phase H₂O+0.1% HCO₂H/CH₃CN+0.1% HCO₂H.

The following tables 1a and 1b comprise respectively specific compounds of formula (I) (name and structure) in accordance with the present disclosure as well their characterization (¹H NMR and liquid chromatography/mass).

TABLE 1a

| Example or | | |
|---|---|---|
| compound | Structure | Name |
| 1 | 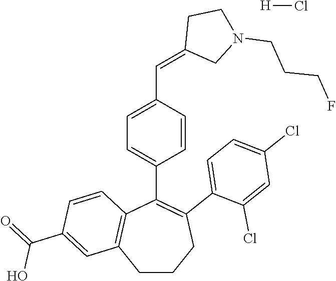 | (Z)-8-(2,4-dichlorophenyl)-9-(4-((1- (3,3-fluoropropyl)pyrrolidin-3- ylidene)methyl)phenyl)-6,7-dihydro- 5H-benzo[7]annulene-3-carboxylic acid, hydrochloride |
| 2 | 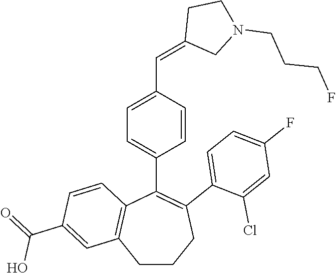 [SMILES: O=C(O)c1ccc2c(c1)CCCC(c1ccc(F)cc1Cl)=C2c1ccc(/C=C2/CCN(CCCF)C2)cc1] | (Z)-8-(2-chloro-4-fluorophenyl)-9-(4- ((1-(3,3-fluoropropyl)pyrrolidin-3- ylidene)methyl)phenyl)-6,7-dihydro- 5H-benzo[7]annulene-3-carboxylic acid |
| 3 | 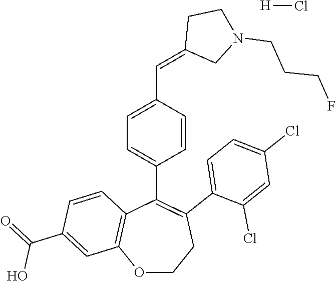 | (Z)-4-(2,4-dichlorophenyl)-5-(4-((1- (3-fluoropropyl)pyrrolidin-3- ylidene)methyl)phenyl)-2,3- dihydrobenzo[b]oxepine-8-carboxylic acid hydrochloride |
| 4 | 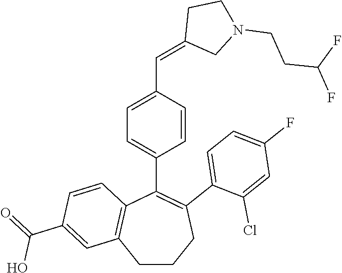 [SMILES: O=C(O)c1ccc2c(c1)CCCC(c1ccc(F)cc1Cl)=C2c1ccc(/C=C2/CCN(CCC(F)F)C2)cc1] | (Z)-8-(2,4-dichlorophenyl)-9-(4-((1- (3,3-difluoropropyl)pyrrolidin-3- ylidene)methyl)phenyl)-6,7-dihydro- 5H-benzo[7]annulene-3-carboxylic acid |
| 5 | 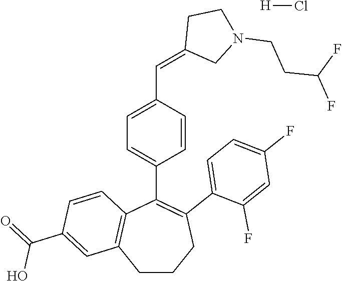 | (Z)-8-(2,4-difluorophenyl)-9-(4-((1- (3,3-difluoropropyl)pyrrolidin-3- ylidene)methyl)phenyl)-6,7-dihydro- 5H-benzo[7]annulene-3-carboxylic acid hydrochloride |
| 6 | 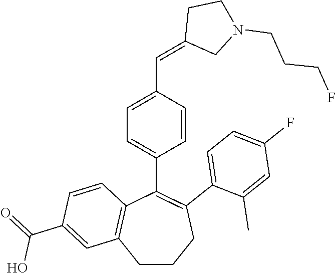 [SMILES: Cc1cc(F)ccc1C1=C(c2ccc(/C=C3/CCN(CCCF)C3)cc2)c2ccc(C(=O)O)cc2CCC1] | (Z)-8-(4-fluoro-2-methylphenyl)-9-(4- ((1-(3-fluoropropyl)pyrrolidin-3- ylidene)methyl)phenyl)-6,7-dihydro- 5H-benzo[7]annulene-3-carboxylic acid |
| 7 | 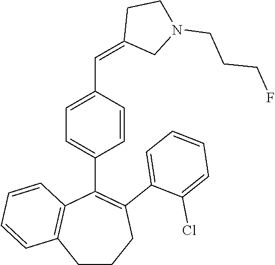 [SMILES: FCCCN1CC/C(=C/c2ccc(C3=C(c4ccccc4Cl)CCCc4ccccc43)cc2)C1] | (Z)-3-(4-(8-(2-chlorophenyl)-6,7- dihydro-5H-benzo[7]annulen-9- yl)benzylidene)-1-(3- fluoropropyl)pyrrolidine |
| 8 | 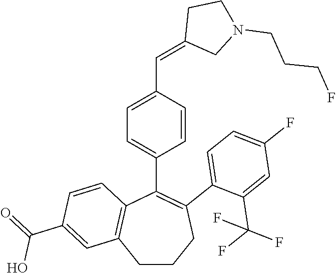 [SMILES: O=C(O)c1ccc2c(c1)CCCC(c1ccc(F)cc1C(F)(F)F)=C2c1ccc(/C=C2/CCN(CCCF)C2)cc1] | (Z)-8-(4-fluoro-2- (trifluoromethyl)phenyl)-9-(4-((1-(3- fluoropropyl)pyrrolidin-3- ylidene)methyl)phenyl)-6,7-dihydro- 5H-benzo[7]annulene-3-carboxylic acid |
| 9 | 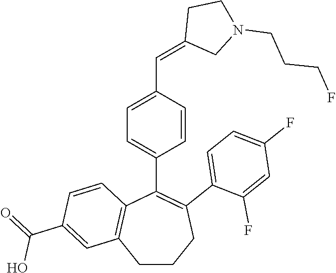 [SMILES: O=C(O)c1ccc2c(c1)CCCC(c1ccc(F)cc1F)=C2c1ccc(/C=C2/CCN(CCCF)C2)cc1] | (Z)-8-(2,4-difluorophenyl)-9-(4-((1- (3,3-fluoropropyl)pyrrolidin-3- ylidene)methyl)phenyl)-6,7-dihydro- 5H-benzo[7]annulene-3-carboxylic |
| 10 | 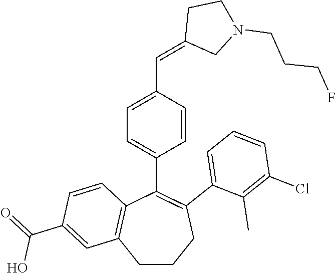 [SMILES: Cc1c(Cl)cccc1C1=C(c2ccc(/C=C3/CCN(CCCF)C3)cc2)c2ccc(C(=O)O)cc2CCC1] | (Z)-8-(3-chloro-2-methylphenyl)-9- (4-((1-(3-fluoropropyl)pyrrolidin-3- ylidene)methyl)phenyl)-6,7-dihydro- 5H-benzo[7]annulene-3-carboxylic acid |
| 11 | 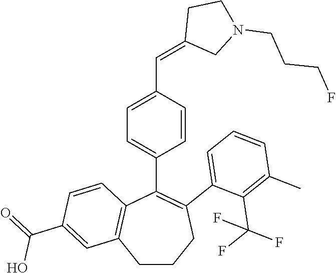 [SMILES: Cc1cccc(C2=C(c3ccc(/C=C4/CCN(CCCF)C4)cc3)c3ccc(C(=O)O)cc3CCC2)c1C(F)(F)F] | (Z)-9-(4-((1-(3- fluoropropyl)pyrrolidin-3- ylidene)methyl)phenyl)-8-(3-methyl- 2-(trifluoromethyl)phenyl)-6,7- dihydro-5H-benzo[7]annulene-3- carboxylic acid |
| 12 | 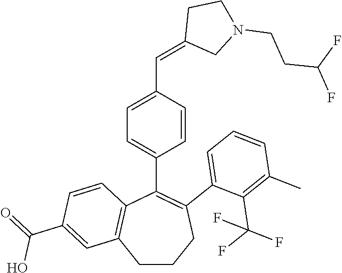 [SMILES: Cc1cccc(C2=C(c3ccc(/C=C4/CCN(CCC(F)F)C4)cc3)c3ccc(C(=O)O)cc3CCC2)c1C(F)(F)F] | (Z)-9-(4-((1-(3,3- difluoropropyl)pyrrolidin-3- ylidene)methyl)phenyl)-8-(3-methyl- 2-(trifluoromethyl)phenyl)-6,7- dihydro-5H-benzo[7]annulene-3- carboxylic acid |
| 13 | 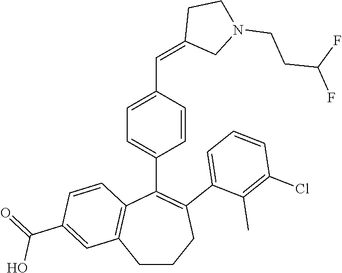 [SMILES: Cc1c(Cl)cccc1C1=C(c2ccc(/C=C3/CCN(CCC(F)F)C3)cc2)c2ccc(C(=O)O)cc2CCC1] | (Z)-8-(3-chloro-2-methylphenyl)-9- (4-((1-(3,3-difluoropropyl)pyrrolidin- 3-ylidene)methyl)phenyl)-6,7- dihydro-5H-benzo[7]annulene-3- carboxylic acid |
| 14 | 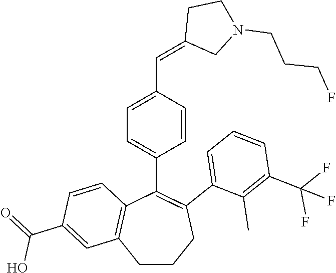 [SMILES: Cc1c(C2=C(c3ccc(/C=C4/CCN(CCCF)C4)cc3)c3ccc(C(=O)O)cc3CCC2)cccc1C(F)(F)F] | (Z)-9-(4-((1-(3- fluoropropyl)pyrrolidin-3- ylidene)methyl)phenyl)-8-(2-methyl- 3-(trifluoromethyl)phenyl)-6,7- dihydro-5H-benzo[7]annulene-3- carboxylic acid |
| 15 | 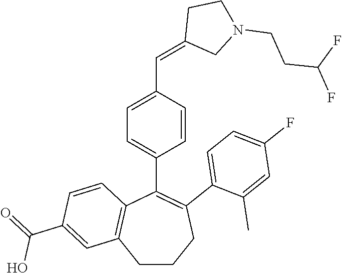 [SMILES: Cc1cc(F)ccc1C1=C(c2ccc(/C=C3/CCN(CCC(F)F)C3)cc2)c2ccc(C(=O)O)cc2CCC1] | (Z)-9-(4-((1-(3,3- difluoropropyl)pyrrolidin-3- ylidene)methyl)phenyl)-8-(4-fluoro-2- methylphenyl)-6,7-dihydro-5H- benzo[7]annulene-3-carboxylic acid |
| 16 | 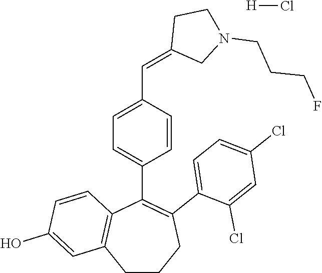 | (Z)-8-(2,4-dichlorophenyl)-9-(4-((1- (3-fluoropropyl)pyrrolidin-3- ylidene)methyl)phenyl)-6,7-dihydro- 5H-benzo[7]annulen-3-ol hydrochloride |
| 17 | 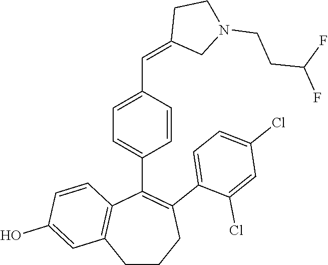 [SMILES: Oc1ccc2c(c1)CCCC(c1ccc(Cl)cc1Cl)=C2c1ccc(/C=C2/CCN(CCC(F)F)C2)cc1] | (Z)-8-(2,4-dichlorophenyl)-9-(4-((1- (3-difluoropropyl)pyrrolidin-3- ylidene)methyl)phenyl)-6,7-dihydro- 5H-benzo[7]annulen-3-ol |
| 18 | 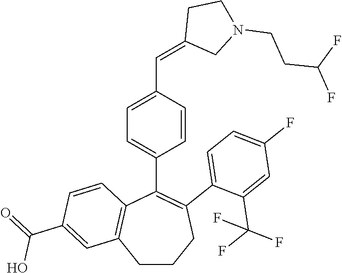 [SMILES: O=C(O)c1ccc2c(c1)CCCC(c1ccc(F)cc1C(F)(F)F)=C2c1ccc(/C=C2/CCN(CCC(F)F)C2)cc1] | (Z)-9-(4-((1-(3,3- difluoropropyl)pyrrolidin-3- ylidene)methyl)phenyl)-8-(4-fluoro-2- (trifluoromethyl)phenyl)-6,7-dihydro- 5H-benzo[7]annulene-3-carboxylic acid |
| 19 | 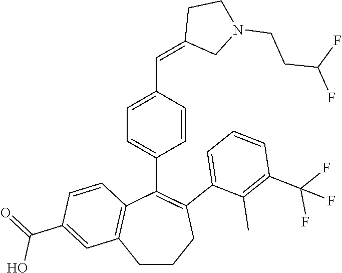 [SMILES: Cc1c(C2=C(c3ccc(/C=C4/CCN(CCC(F)F)C4)cc3)c3ccc(C(=O)O)cc3CCC2)cccc1C(F)(F)F] | (Z)-9-(4-((1-(3,3- difluoropropyl)pyrrolidin-3- ylidene)methyl)phenyl)-8-(2-methyl- 3-(trifluoromethyl)phenyl)-6,7- dihydro-5H-benzo[7]annulene-3- carboxylic acid |
| 20 | 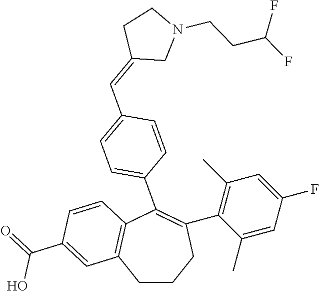 [SMILES: Cc1cc(F)cc(C)c1C1=C(c2ccc(/C=C3/CCN(CCC(F)F)C3)cc2)c2ccc(C(=O)O)cc2CCC1] | (Z)-9-(4-((1-(3,3- difluoropropyl)pyrrolidin-3- ylidene)methyl)phenyl)-8-(4-fluoro- 2,6-dimethyl)phenyl)-6,7-dihydro-5H- benzo[7]annulene-3-carboxylic acid |
| 21 | 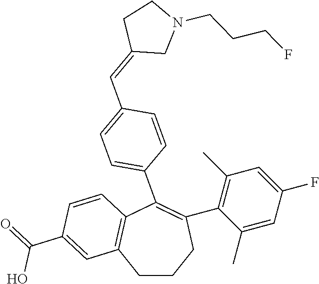 [SMILES: Cc1cc(F)cc(C)c1C1=C(c2ccc(/C=C3/CCN(CCCF)C3)cc2)c2ccc(C(=O)O)cc2CCC1] | (Z)-8-(4-fluoro-2,6-dimethylphenyl)- 9-(4-((1-(3-fluoropropyl)pyrrolidin-3- ylidene)methyl)phenyl)-6,7-dihydro- 5H-benzo[7]annulene-3-carboxylic acid |
| 22 | 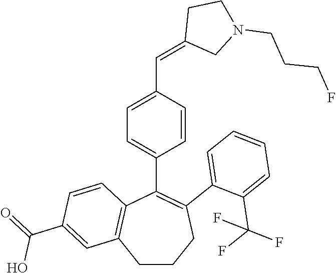 [SMILES: O=C(O)c1ccc2c(c1)CCCC(c1ccccc1C(F)(F)F)=C2c1ccc(/C=C2/CCN(CCCF)C2)cc1] | (Z)-9-(4-((1-(3- fluoropropyl)pyrrolidin-3- ylidene)methyl)phenyl)-8-(2- (trifluoromethyl)phenyl)-6,7-dihydro- 5H-benzo[7]annulene-3-carboxylic acid |
| 23 | 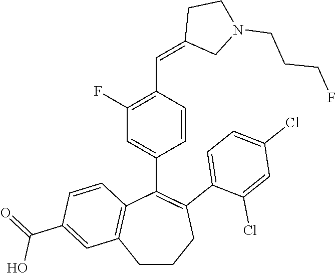 [SMILES: O=C(O)c1ccc2c(c1)CCCC(c1ccc(Cl)cc1Cl)=C2c1ccc(/C=C2/CCN(CCCF)C2)c(F)c1] | (Z)-8-(2,4-dichlorophenyl)-9-(3- fluoro-4-((1-(3- fluoropropyl)pyrrolidin-3- ylidene)methyl)phenyl)-6,7-dihydro- 5H-benzo[7]annulene-3-carboxylic acid |
| 24 | 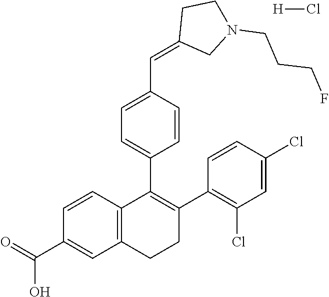 | (Z)-6-(2,4-dichlorophenyl)-5-(4-((1- (3-fluoropropyl)pyrrolidin-3- ylidene)methyl)phenyl)-7,8- dihydronaphthalene-2-carboxylic acid hydrochloride |
| 25 | 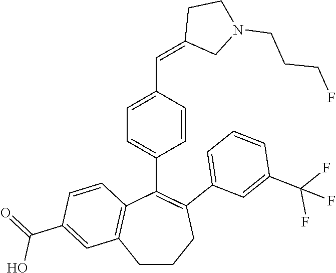 [SMILES: O=C(O)c1ccc2c(c1)CCCC(c1cccc(C(F)(F)F)c1)=C2c1ccc(/C=C2/CCN(CCCF)C2)cc1] | (Z)-9-(4-((1-(3- fluoropropyl)pyrrolidin-3- ylidene)methyl)phenyl)-8-(3- (trifluoromethyl)phenyl)-6,7-dihydro- 5H-benzo[7]annulene-3-carboxylic acid |
| 26 | 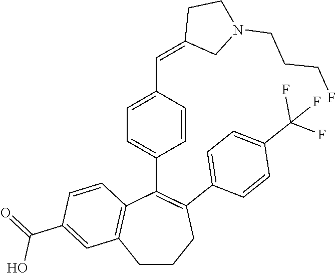 [SMILES: O=C(O)c1ccc2c(c1)CCCC(c1ccc(C(F)(F)F)cc1)=C2c1ccc(/C=C2/CCN(CCCF)C2)cc1] | (Z)-9-(4-((1-(3- fluoropropyl)pyrrolidin-3- ylidene)methyl)phenyl)-8-(4- (trifluoromethyl)phenyl)-6,7-dihydro- 5H-benzo[7]annulene-3-carboxylic acid |
| 27 | 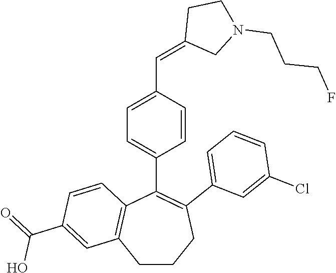 [SMILES: O=C(O)c1ccc2c(c1)CCCC(c1cccc(Cl)c1)=C2c1ccc(/C=C2/CCN(CCCF)C2)cc1] | (Z)-8-(3-chlorophenyl)-9-(4-((1-(3- fluoropropyl)pyrrolidin-3- ylidene)methyl)phenyl)-6,7-dihydro- 5H-benzo[7]annulene-3-carboxylic acid |
| 28 | 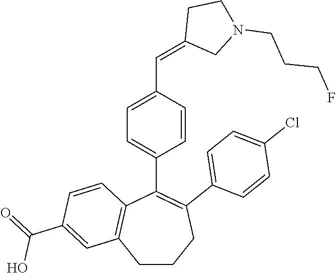 [SMILES: O=C(O)c1ccc2c(c1)CCCC(c1ccc(Cl)cc1)=C2c1ccc(/C=C2/CCN(CCCF)C2)cc1] | (Z)-8-(4-chlorophenyl)-9-(4-((1-(3- fluoropropyl)pyrrolidin-3- ylidene)methyl)phenyl)-6,7-dihydro- 5H-benzo[7]annulene-3-carboxylic acid |
| 29 | 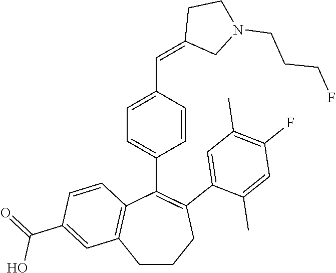 [SMILES: Cc1cc(C2=C(c3ccc(/C=C4/CCN(CCCF)C4)cc3)c3ccc(C(=O)O)cc3CCC2)c(C)cc1F] | (Z)-8-(4-fluoro-2,5-dimethylphenyl)- 9-(4-((1-(3-fluoropropyl)pyrrolidin-3- ylidene)methyl)phenyl)-6,7-dihydro- 5H-benzo[7]annulene-3-carboxylic acid |
| 30 | 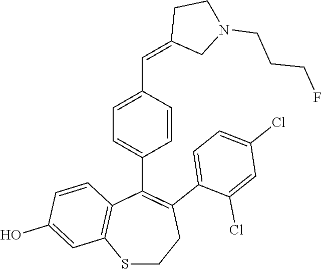 [SMILES: Oc1ccc2c(c1)SCCC(c1ccc(Cl)cc1Cl)=C2c1ccc(/C=C2/CCN(CCCF)C2)cc1] | (Z)-4-(2,4-dichlorophenyl)-5-(4-((1- (3-fluoropropyl)pyrrolidin-3- ylidene)methyl)phenyl)-2,3- dihydrobenzo[b]thiepin-8-ol |

TABLE 1b

| | | | MASS: |
|---|---|---|---|
| Example | | | LC/MS |
| or | Preparation | | (m/z, |
| compound | Method | NMR | MH+): |
| 1 | A | 1H NMR (400 MHz, DMSO-d6) δ ppm 2.01-2.30 | 550 |
| | | (m, 6 H), 2.52-3.29 (m, 7 H), 3.57-3.82 (m, 1 | |
| | | H), 3.87-4.16 (m, 1 H), 4.31-4.46 (m, 1 H), 4.55 | |
| | | (dt, J = 47, 6 Hz, 2 H), 6.44 (br s, 1 H), 6.79-6.92 | |
| | | (m, 3 H), 7.08 (d, J = 8 Hz, 2 H), 7.16-7.35 (m, 2 | |
| | | H), 7.60 (d, J = 2 Hz, 1 H), 7.76 (dd, J = 8, 2 Hz, 1 | |
| | | H), 7.94 (d, J = 2 Hz, 1 H), 10.28 (s, 1 H), 12.95 (s, | |
| | | 1 H) | |
| 2 | B | 1H NMR (400 MHz, DMSO-d6) δ ppm 1.74-1.90 | 534 |
| | | (m, 2 H), 2.06-2.29 (m, 4 H), 2.52-2.61 (m, 6 H), | |
| | | 2.88-3.03 (m, 2 H), 3.17-3.27 (m hidden, 2 H), | |
| | | 4.48 (dt, J = 48, 6 Hz, 2 H), 6.19 (s, 1 H), 6.80 (d, | |
| | | J = 8 Hz, 2 H), 6.87 (d, J = 8 Hz, 1 H), 6.99 (d, J = 8 | |
| | | Hz, 2 H), 7.07 (td, J = 8, 3 Hz, 1 H), 7.23 (br dd, J = 9, | |
| | | 6 Hz, 1 H), 7.41 (dd, J = 9, 3 Hz, 1 H), 7.75 (dd, J = 8, | |
| | | 2 Hz, 1 H), 7.92 (d, J = 2 Hz, 1 H), 11.17-13.77 (m, | |
| | | 1 H) | |
| 3 | A | 1H NMR (400 MHz, DMSO-d6) δ ppm 1.93-2.21 | 552 |
| | | (m, 2 H), 2.40-2.85 (m, 6 H), 3.17-3.29 (m | |
| | | hidden, 4 H), 4.39-4.69 (m, 4 H), 6.41 (br s, 1 H), | |
| | | 6.84 (d, J = 8 Hz, 1 H), 6.90 (d, J = 8 Hz, 2 H), 7.06- | |
| | | 7.16 (m, 3 H), 7.26 (dd, J = 8, 2 Hz, 1 H), 7.58-7.67 | |
| | | (m, 3 H), 10.44 (br s, 1 H), 13.08 (s, 1 H) | |
| 4 | A | 1H NMR (400 MHz, DMSO-d6) δ ppm 1.96-2.12 | 568 |
| | | (m, 2 H), 2.13-2.29 (m, 4 H), 2.53-2.64 (m, 6 H), | |
| | | 2.87-3.00 (m, 2 H), 3.39-3.50 (m, 2 H), 6.10 (tt, | |
| | | J = 56, 5 Hz, 1 H), 6.20 (s, 1 H), 6.81 (d, J = 9 Hz, 2 | |
| | | H), 6.87 (d, J = 8 Hz, 1 H), 7.01 (d, J = 9 Hz, 2 H), | |
| | | 7.18-7.23 (m, 1 H), 7.27 (dd, J = 7, 2 Hz, 1 H), 7.59 | |
| | | (d, J = 2 Hz, 1 H), 7.75 (dd, J = 8, 2 Hz, 1 H), 7.92 (d, | |
| | | J = 2 Hz, 1 H), 11.92-14.00 (m, 1 H) | |
| 5 | A | 1H NMR (400 MHz, DMSO-d6) δ ppm 2.02-2.27 | 536 |
| | | (m, 4 H), 2.30-3.44 (m, 9 H), 3.62-3.82 (m, 1 H), | |
| | | 3.91-4.13 (m, 1 H), 4.32-4.59 (m, 1 H), 6.24 (tt, | |
| | | J = 56, 4 Hz, 1 H), 6.45 (br s, 1 H), 6.78-6.91 (m, 3 | |
| | | H), 6.98 (td, J = 8, 2 Hz, 1 H), 7.08 (d, J = 9 Hz, 2 H), | |
| | | 7.14 (m, 1 H), 7.29 (td, J = 9, 7 Hz, 1 H), 7.76 (dd, | |
| | | J = 8, 2 Hz, 1 H), 7.93 (d, J = 2 Hz, 1 H), 10.59 (br s, 1 | |
| | | H), 12.86 (br s, 1 H) | |
| 6 | A | 1H NMR (400 MHz, DMSO-d6) δ ppm 1.73-1.94 | 514 |
| | | (m, 2 H), 2.12-2.17 (m, 4 H), 2.18 (s, 3 H), 2.52- | |
| | | 2.61 (m, 6 H), 2.84-2.99 (m, 2 H), 3.23-3.32 (m, | |
| | | 2 H), 4.48 (dt, J = 47, 6 Hz, 2 H), 6.18 (s, 1 H), 6.74 | |
| | | (d, J = 9 Hz, 2 H), 6.87 (d, J = 8 Hz, 2 H), 6.92-7.00 | |
| | | (m, 3 H), 7.07 (dd, J = 8, 6 Hz, 1 H), 7.74 (dd, J = 8, 2 | |
| | | Hz, 1 H), 7.91 (d, J = 2 Hz, 1 H) | |
| 7 | A | 1H NMR (400 MHz, DMSO-d6) δ ppm 1.85 (dt, | 472 |
| | | J = 25, 7 Hz, 2 H), 2.05-2.28 (m, 4 H), 2.53-2.65 | |
| | | (m, 5 H), 2.81-2.98 (m, 2 H), 3.32-3.40 (m, 2 H), | |
| | | 4.48 (dt, J = 47, 6 Hz, 2 H), 6.19 (br s, 1 H), 6.77 (dd, | |
| | | J = 8 Hz, 1 H), 6.81 (d, J = 8 Hz, 2 H), 6.96 (d, J = 8 | |
| | | Hz, 2 H), 7.13-7.28 (m, 6 H), 7.34 (dd, J = 8, 2 Hz, | |
| | | 1 H), 7.42 (m, 1 H) | |
| 8 | B | 1H NMR (400 MHz, DMSO-d6) δ ppm 1.74-1.96 | 568 |
| | | (m, 2 H), 2.19 (m, J = 2 Hz, 4 H), 2.51-2.64 (m, 6 | |
| | | H), 2.81-3.05 (m, 2 H), 3.23-3.30 (m hidden, 2 | |
| | | H), 4.47 (dt, J = 47, 6 Hz, 2 H), 6.18 (s, 1 H), 6.78 (d, | |
| | | J = 8 Hz, 2 H), 6.87 (d, J = 8 Hz, 1 H), 6.99 (d, J = 9 | |
| | | Hz, 2 H), 7.27 (dd, J = 9, 6 Hz, 1 H), 7.35 (td, J = 8, 3 | |
| | | Hz, 1 H), 7.63 (dd, J = 9, 3 Hz, 1 H), 7.75 (dd, J = 8, 2 | |
| | | Hz, 1 H), 7.91 (d, J = 2 Hz, 1 H) | |
| 9 | A | 1H NMR (400 MHz, DMSO-d6) δ ppm 1.81-2.04 | 518 |
| | | (m, 2 H), 2.09-2.28 (m, 4 H), 2.58-2.96 (m, 8 H), | |
| | | 3.48-3.67 (m, 2 H), 4.50 (dt, J = 47, 6 Hz, 2 H), | |
| | | 6.28 (br s, 1 H), 6.82 (d, J = 8 Hz, 2 H), 6.87 (d, J = 8 | |
| | | Hz, 1 H), 6.91-6.99 (m, 1 H), 7.03 (d, J = 8 Hz, 2 | |
| | | H), 7.12 (ddd, J = 11, 9, 3 Hz, 1 H), 7.27 (td, J = 9, 7 | |
| | | Hz, 1 H), 7.75 (dd, J = 8, 2 Hz, 1 H), 7.92 (d, J = 2 Hz, | |
| | | 1 H), 10.35-14.20 (m, 1 H) | |
| 10 | B | 1H NMR (400 MHz, DMSO-d6) δ ppm 1.76-1.91 | 530 |
| | | (m, 2 H), 2.09-2.20 (m, 4 H), 2.24 (s, 3 H), 2.52- | |
| | | 2.61 (m, 6 H), 2.82-3.01 (m, 2 H), 3.20-3.26 (m | |
| | | hidden, 2 H), 4.48 (dt, J = 47, 6 Hz, 2 H), 6.18 (s, 1 | |
| | | H), 6.75 (d, J = 8 Hz, 2 H), 6.86 (d, J = 8 Hz, 1 H), | |
| | | 6.97 (d, J = 9 Hz, 2 H), 6.99-7.02 (m, 1 H), 7.06 (t, | |
| | | J = 8 Hz, 1 H), 7.25 (dd, J = 8, 2 Hz, 1 H), 7.74 (dd, | |
| | | J = 8, 2 Hz, 1 H), 7.91 (d, J = 2 Hz, 1 H), 10.46- | |
| | | 14.95 (m, 1 H) | |
| 11 | B | 1H NMR (400 MHz, DMSO-d6) δ ppm 1.73-1.95 | 564 |
| | | (m, 2 H), 2.00-2.29 (m, 4 H), 2.43-2.48 (m, 2 H), | |
| | | 2.50-2.51 (m hidden, 3 H), 2.52-2.57 (m, 4 H), | |
| | | 2.83 (m, 1 H), 3.00 (m, 1 H), 3.27-3.29 (m hidden, | |
| | | 2 H), 4.47 (dt, J = 47, 6 Hz, 2 H), 6.17 (s, 1 H), 6.81 | |
| | | (d, J = 8 Hz, 2 H), 6.86 (d, J = 8 Hz, 1 H), 6.91-7.04 | |
| | | (m, 3 H), 7.22-7.30 (m, 2 H), 7.74 (dd, J = 8, 2 Hz, | |
| | | 1 H), 7.90 (d, J = 2 Hz, 1 H) | |
| 12 | B | 1H NMR (400 MHz, DMSO-d6) δ ppm 1.91-2.27 | 582 |
| | | (m, 6 H), 2.43-2.62 (m partially hidden, 9 H), 2.83 | |
| | | (m, 1 H), 3.00 (m, 1 H), 3.32-3.35 (m hidden, 2 | |
| | | H), 6.07 (tt, J = 57, 5 Hz, 1 H), 6.17 (s, 1 H), 6.81 (d, | |
| | | J = 8 Hz, 2 H), 6.85 (d, J = 8 Hz, 1 H), 6.92-7.05 (m, | |
| | | 3 H), 7.21-7.30 (m, 2 H), 7.74 (dd, J = 8, 2 Hz, 1 | |
| | | H), 7.90 (d, J = 2 Hz, 1 H) | |
| 13 | B | 1H NMR (500 MHz, DMSO-d6) δ ppm 1.95-2.10 | 548 |
| | | (m, 2 H), 2.17 (m, 7 H), 2.52-2.61 (m, 6 H), 2.80- | |
| | | 3.02 (m, 2 H), 3.33 (br s, 2 H), 6.09 (tt, J = 57, 5 Hz, | |
| | | 1 H), 6.18 (m, 1 H), 6.75 (d, J = 8 Hz, 2 H), 6.88 (d, | |
| | | J = 8 Hz, 1 H), 6.96-7.04 (m, 3 H), 7.06 (t, J = 9 Hz, | |
| | | 1 H), 7.25 (dd, J = 8, 1 Hz, 1 H), 7.75 (dd, J = 8, 2 Hz, | |
| | | 1 H), 7.92 (d, J = 2 Hz, 1 H), 12.87 (br s, 1 H) | |
| 14 | B | 1H NMR (400 MHz, DMSO-d6) δ ppm 1.71-1.92 | 564 |
| | | (m, 2 H), 2.11-2.27 (m, 4 H), 2.30-2.36 (m, 3 H), | |
| | | 2.51-2.58 (m, 6 H), 2.82-3.03 (m, 2 H), 3.28 (br | |
| | | s, 2 H), 4.47 (dt, J = 47, 6 Hz, 2 H), 6.18 (m, 1 H), | |
| | | 6.74 (d, J = 8 Hz, 2 H), 6.85 (d, J = 8 Hz, 1 H), 6.95 | |
| | | (d, J = 8 Hz, 2 H), 7.24 (t, J = 8 Hz, 1 H), 7.34 (d, J = 7 | |
| | | Hz, 1 H), 7.51 (d, J = 8 Hz, 1 H), 7.73 (dd, J = 8, 2 Hz, | |
| | | 1 H), 7.90 (d, J = 2 Hz, 1 H) | |
| 15 | B | 1H NMR (400 MHz, DMSO-d6) δ ppm 1.93-2.11 | 532 |
| | | (m, 2 H), 2.11-2.26 (m, 7 H), 2.50-2.63 (m, 6 H), | |
| | | 2.81-3.02 (m, 2 H), 3.31 (m hidden, 2 H), 6.07 (tt, | |
| | | J = 57, 4 Hz, 1 H), 6.17 (m, 1 H), 6.74 (d, J = 9 Hz, 2 | |
| | | H), 6.86 (d, J = 8 Hz, 2 H), 6.93-7.01 (m, 3 H), 7.07 | |
| | | (dd, J = 8, 6 Hz, 1 H), 7.74 (dd, J = 8, 2 Hz, 1 H), 7.91 | |
| | | (d, J = 2 Hz, 1 H) | |
| 16 | D | 1H NMR (500 MHz, DMSO-d6) δ ppm 2.03-2.16 | 522 |
| | | (m, 4 H), 2.18-2.25 (m, 2 H), 2.71-2.90 (m, 4 H), | |
| | | 3.42 (m hidden, 4 H), 4.25-4.45 (m, 2 H), 4.54 (dt, | |
| | | J = 47, 6 Hz, 2 H), 6.44 (br s, 1 H), 6.54 (d, J = 8 Hz, 1 | |
| | | H), 6.58 (dd, J = 8, 3 Hz, 1 H), 6.75 (d, J = 3 Hz, 1 H), | |
| | | 6.86 (d, J = 8 Hz, 2 H), 7.04 (d, J = 8 Hz, 2 H), 7.17 | |
| | | (d, J = 8 Hz, 1 H), 7.24 (dd, J = 8, 3 Hz, 1 H), 7.56 (d, | |
| | | J = 2 Hz, 1 H), 9.54 (s, 1 H), 9.92-10.32 (m, 1 H) | |
| 17 | D | 1H NMR (500 MHz, DMSO-d6) δ ppm 1.97-2.14 | 540 |
| | | (m, 4 H), 2.17-2.25 (m, 2 H), 2.53-2.63 (m, 6 H), | |
| | | 2.68-2.85 (m, 2 H), 3.28-3.35 (m hidden , 2 H), | |
| | | 6.09 (tt, J = 57, 4 Hz, 1 H), 6.17-6.20 (m, 1 H), 6.50- | |
| | | 6.64 (m, 2 H), 6.73 (d, J = 2 Hz, 1 H), 6.80 (d, J = 8 | |
| | | Hz, 2 H), 6.98 (d, J = 8 Hz, 2 H), 7.15 (d, J = 8 Hz, 1 | |
| | | H), 7.22 (dd, J = 8, 2 Hz, 1 H), 7.54 (d, J = 2 Hz, 1 H), | |
| | | 9.51 (br s, 1 H) | |
| 18 | B | 1H NMR (500 MHz, DMSO-d6) δ ppm 1.98-2.11 | 586 |
| | | (m, 2 H), 2.13-2.25 (m, 4 H), 2.53-2.62 (m, 6 H), | |
| | | 2.80-2.92 (m, 1 H), 2.95-3.05 (m, 1 H), 3.33 (m | |
| | | hidden, 2 H), 6.09 (tt, J = 57, 4 Hz, 1 H), 6.18 (br s, 1 | |
| | | H), 6.79 (d, J = 8 Hz, 2 H), 6.87 (d, J = 8 Hz, 1 H), | |
| | | 7.00 (d, J = 8 Hz, 2 H), 7.27 (dd, J = 8, 6 Hz, 1 H), | |
| | | 7.34 (td, J = 8, 3 Hz, 1 H), 7.63 (dd, J = 9, 3 Hz, 1 H), | |
| | | 7.75 (dd, J = 8, 2 Hz, 1 H), 7.91 (d, J = 2 Hz, 1 H) | |
| 19 | B | 1H NMR (500 MHz, DMSO-d6) δ ppm 2.09-2.42 | 582 |
| | | (m, 9 H), 2.72-2.84 (m, 2 H), 2.85-3.07 (m, 2 H), | |
| | | 3.25-3.32 (m hidden, 2 H), 3.94-4.55 (m hidden, | |
| | | 4 H), 6.21 (br tt, J = 56, 4 Hz, 1 H), 6.42 (br s, 1 H), | |
| | | 6.80 (m, J = 9 Hz, 2 H), 6.88 (d, J = 8 Hz, 1 H), 7.03 | |
| | | (m, J = 9 Hz, 2 H), 7.25 (t, J = 8 Hz, 1 H), 7.34 (br d, | |
| | | J = 8 Hz, 1 H), 7.53 (d, J = 8 Hz, 1 H), 7.77 (dd, J = 8, 2 | |
| | | Hz, 1 H), 7.95 (d, J = 2 Hz, 1 H), 10.35 (br s, 1 H) | |
| 20 | B | 1H NMR (500 MHz, DMSO-d6) δ ppm 1.96-2.28 | 546 |
| | | (m, 12 H), 2.52-2.63 (m, 6 H), 2.96 (t, J = 7 Hz, 2 | |
| | | H), 3.34 (s, 2 H), 6.09 (tt, J = 57, 4 Hz, 1 H), 6.19 (t, | |
| | | J = 3 Hz, 1 H), 6.71 (d, J = 9 Hz, 2 H), 6.82 (d, J = 10 | |
| | | Hz, 2 H), 6.85 (d, J = 8 Hz, 1 H), 6.97 (d, J = 8 Hz, 2 | |
| | | H), 7.74 (dd, J = 8, 2 Hz, 1 H), 7.93 (d, J = 2 Hz, 1 H) | |
| 21 | B | 1H NMR (400 MHz, DMSO-d6) δ ppm 1.75-1.92 | 528 |
| | | (m, 2 H), 2.06-2.24 (m, 10 H), 2.52-2.58 (m, 6 | |
| | | H), 2.95 (br t, J = 7 Hz, 2 H), 3.31 (m hidden, 2 H), | |
| | | 4.47 (dt, J = 47, 6 Hz, 2 H), 6.18 (t, J = 3 Hz, 1 H), | |
| | | 6.71 (d, J = 9 Hz, 2 H), 6.77-6.86 (m, 3 H), 6.96 (d, | |
| | | J = 8 Hz, 2 H), 7.73 (dd, J = 8, 2 Hz, 1 H), 7.92 (d, J = 2 | |
| | | Hz, 1 H) | |
| 22 | B | 1H NMR (400 MHz, DMSO-d6) δ ppm 1.73-1.93 | 550 |
| | | (m, 2 H), 2.08-2.29 (m, 4 H), 2.51-2.58 (m, 6 H), | |
| | | 2.81-3.08 (m, 2 H), 3.30 (m hidden, 2 H), 4.48 (dt, | |
| | | J = 47, 6 Hz, 2 H), 6.17 (t, J = 3 Hz, 1 H), 6.79 (d, J = 9 | |
| | | Hz, 2 H), 6.87 (d, J = 8 Hz, 1 H), 6.96 (d, J = 9 Hz, 2 | |
| | | H), 7.21 (m, 1 H), 7.36-7.52 (m, 2 H), 7.65-7.80 | |
| | | (m, 2 H), 7.91 (d, J = 2 Hz, 1 H) | |
| 23 | A | 1H NMR (400 MHz, DMSO-d6) δ ppm 1.78-1.91 | 568 |
| | | (m, 2 H), 2.12-2.26 (m, 4 H), 2.56-2.61 (m, 8 H | |
| | | partially hidden), 2.94 (m, 2 H), 4.48 (dt, J = 6 Hz, | |
| | | J = 48 Hz, 2 H), 6.29 (br s, 1 H), 6.60 (dd, J = 12, 2 | |
| | | Hz, 1 H), 6.67 (dd, J = 8, 2 Hz, 1 H), 6.92 (d, J = 8 | |
| | | Hz, 1 H), 7.11 (t, J = 8 Hz, 1 H), 7.26 (d, J = 8 Hz, 1 | |
| | | H), 7.31 (dd, J = 8, 3 Hz, 1 H), 7.62 (d, J = 2 Hz, 1 H), | |
| | | 7.77 (dd, J = 8, 2 Hz, 1 H), 7.93 (d, J = 2 Hz, 1 H), | |
| | | 12.99 (br s, 1 H) | |
| 24 | C | 1H NMR (400 MHz, DMSO-d6) δ ppm 2.00-2.24 | 536 |
| | | (m, 2 H), 2.54 (m hidden, 2 H), 2.58 (m, 1 H), 2.67- | |
| | | 2.77 (m, 2 H), 2.77-2.94 (m, 2 H), 3.07 (br t, J = 8 | |
| | | Hz, 2 H), 3.16(m, 1 H), 3.71 (m, 1 H), 3.91-4.14 | |
| | | (m, 1 H), 4.55 (dt, J = 47, 6 Hz, 2 H), 6.47 (br s, 1 | |
| | | H), 6.74 (d, J = 8 Hz, 1 H), 7.03-7.27 (m, 6 H), 7.55 | |
| | | (d, J = 2 Hz, 1 H), 7.69 (dd, J = 8, 2 Hz, 1 H), 7.85 (d, | |
| | | J = 1 Hz, 1 H), 10.35-10.70 (br s, 1 H), 12.56- | |
| | | 13.10 (br s, 1 H) | |
| 25 | B | 1H NMR (400 MHz, DMSO-d6) δ ppm 1.72-1.94 | 550 |
| | | (m, 2 H), 2.10-2.24 (m, 2 H), 2.32-2.37 (m, 2 H), | |
| | | 2.52-2.62 (m, 6 H), 2.88 (br t, J = 7 Hz, 2 H), 3.28 | |
| | | (m hidden, 2 H), 4.47 (dt, J = 47, 6 Hz, 2 H), 6.21 (t, | |
| | | J = 3 Hz, 1 H), 6.81 (d, J = 8 Hz, 2 H), 6.87 (d, J = 8 | |
| | | Hz, 1 H), 7.00 (d, J = 8 Hz, 2 H), 7.38-7.56 (m, 4 | |
| | | H), 7.74 (dd, J = 8, 2 Hz, 1 H), 7.91 (d, J = 2 Hz, 1 H) | |
| 26 | B | 1H NMR (400 MHz, DMSO-d6) δ ppm 1.69-1.93 | 550 |
| | | (m, 2 H), 2.07-2.20 (m, 2 H), 2.28-2.33 (m, 2 H), | |
| | | 2.51 (m, 6 H), 2.86 (t, J = 7 Hz, 2 H), 3.29-3.32 (m, | |
| | | 2 H), 4.47 (dt, J = 48, 6 Hz, 2 H), 6.21 (t, J = 3 Hz, 1 | |
| | | H), 6.82 (dd, J = 8, 3 Hz, 3 H), 7.00 (d, J = 8 Hz, 2 H), | |
| | | 7.39 (br d, J = 8 Hz, 2 H), 7.55 (d, J = 8 Hz, 2 H), 7.73 | |
| | | (dd, J = 8, 2 Hz, 1 H), 7.90 (d, J = 2 Hz, 1 H) | |
| 27 | B | 1H NMR (400 MHz, DMSO-d6) δ ppm 1.72-1.93 | 516 |
| | | (m, 2 H), 2.04-2.20 (m, 2 H), 2.21-2.31 (m, 2 H), | |
| | | 2.52-2.62 (m, 6 H), 2.85 (t, J = 7 Hz, 2 H), 3.33- | |
| | | 3.33 (m, 2 H), 4.48 (dt, J = 47, 6 Hz, 2 H), 6.22 (t, | |
| | | J = 3 Hz, 1 H), 6.84 (dd, J = 8, 4 Hz, 3 H), 7.02 (d, J = 8 | |
| | | Hz, 2 H), 7.07-7.29 (m, 4 H), 7.73 (dd, J = 8, 2 Hz, | |
| | | 1 H), 7.90 (d, J = 2 Hz, 1 H) | |
| 28 | B | 1H NMR (400 MHz, DMSO-d6) δ ppm 1.72-1.94 | 516 |
| | | (m, 2 H), 2.07-2.19 (m, 2 H), 2.22-2.31 (m, 2 H), | |
| | | 2.51-2.61 (m, 6 H), 2.85 (t, J = 7 Hz, 2 H), 3.31 (m | |
| | | hidden, 2 H), 4.48 (dt, J = 48, 6 Hz, 2 H), 6.22 (t, J = 3 | |
| | | Hz, 1 H), 6.82 (d, J = 8 Hz, 2 H), 6.85 (d, J = 8 Hz, 1 | |
| | | H), 7.01 (d, J = 8 Hz, 2 H), 7.16-7.21 (m, 2 H), 7.23- | |
| | | 7.30 (m, 2 H), 7.73 (dd, J = 8, 2 Hz, 1 H), 7.90 (d, | |
| | | J = 2 Hz, 1 H) | |
| 29 | B | 1H NMR (400 MHz, DMSO-d6) δ ppm 1.74-1.95 | 528 |
| | | (m, 2 H), 2.04-2.23 (m, 10 H), 2.53-2.61 (m | |
| | | partially hidden, 6 H), 2.81-2.98 (m, 2 H), 3.31 (br | |
| | | hidden, 2 H), 4.48 (dt, J = 47, 6 Hz, 2 H), 6.19 (br s, | |
| | | 1 H), 6.75 (d, J = 8 Hz, 2 H), 6.85 (d, J = 8 Hz, 1 H), | |
| | | 6.88 (d, J = 11 Hz, 1 H), 6.94-7.01 (m, 3 H), 7.73 | |
| | | (dd, J = 8, 2 Hz, 1 H), 7.90 (d, J = 2 Hz, 1 H) | |
| 30 | E | 1H NMR (500 MHz, DMSO-d6) δ ppm 1.75-1.92 | 540 |
| | | (m, 2 H), 2.37-2.60 (m, 8 H), 3.13 (td, J = 12, 5 Hz, | |
| | | 1 H), 3.25-3.37 (m, 2 H), 3.41-3.48 (m, 1 H), | |
| | | 4.48 (dt, J = 48, 6 Hz, 2 H), 6.19 (br s, 1 H), 6.33 (d, | |
| | | J = 3 Hz, 1 H), 6.71 (dd, J = 8, 3 Hz, 1 H), 6.80 (d, J = 8 | |
| | | Hz, 2 H), 7.02 (d, J = 8 Hz, 2 H), 7.09 (d, J = 8 Hz, 1 | |
| | | H), 7.25 (dd, J = 8, 2 Hz, 1 H), 7.45 (d, J = 8 Hz, 1 H), | |
| | | 7.66 (d, J = 2 Hz, 1 H), 9.59 (s, 1 H) | |

The examples which follow describe the preparation of some compounds of formula (I) described herein. The numbers of the compounds exemplified below match those given in the Tables 1a and 1b above. All reactions are performed under inert atmosphere, unless otherwise stated.

In the following examples, when the source of the starting products is not specified, it should be understood that said products are known compounds.

### INTERMEDIATES

### Intermediate 1: Methyl 9-(4,4,5,5-tetramethyl-1,3,2-dioxaborolan-2-yl)-6,7-dihydro-5H-benzo [7]annulene-3-carboxylate

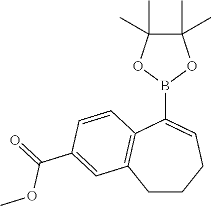

A mixture of methyl 9-(((trifluoromethyl)sulfonyl)oxy)-6,7-dihydro-5H-benzo[7]annulene-3-carboxylate (15 g, 42.82 mmol) (prepared according to WO2017140669), in toluene (150 ml), Pd(PPh₃)₂Cl₂ (1.53 g, 2.14 mmol), PPh₃ (673.87 mg, 2.57 mmol), bis(pinacolato)diboron (13.38 g, 52.67 mmol) and PhOK (8.04 g, 60.80 mmol) was heated to 75° C. for 1.5 h. The yellow suspension becomes orange then brown. After cooling to RT, DCM (150 mL) and water (150 mL) were added, and decantation was done by hydrophobic column. The organic phase was concentrated under reduced pressure. The residue obtained was purified by flash chromatography, eluting with a gradient of heptane/DCM: from 85/15 to 20/80 to give 10.1 g (72%) of methyl 9-(4,4,5,5-tetramethyl-1,3,2-dioxaborolan-2-yl)-6,7-dihydro-5H-benzo[7]annulene-3-carboxylate as a white solid. LC/MS (m/z, MH+): 329

### Intermediate 2: Methyl 8-(2,4-dichlorophenyl)-9-(4,4,5,5-tetramethyl-1,3,2-dioxaborolan-2-yl)-6,7-dihydro-5H-benzo[7]annulene-3-carboxylate

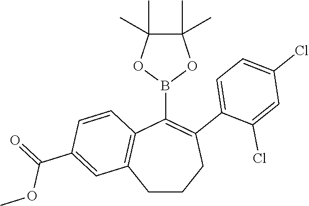 [SMILES: COC(=O)c1ccc2c(c1)CCCC(c1ccc(Cl)cc1Cl)=C2B1OC(C)(C)C(C)(C)O1]

Intermediate 2 was prepared following a similar procedure to that of Intermediate 1 from methyl 8-(2,4-dichlorophenyl)-9-(((trifluoromethyl)sulfonyl)oxy)-6,7-dihydro-5H-benzo[7]annulene-3-carboxylate (prepared according to WO2020/049153) to give 3.9 g (82%) of methyl 8-(2,4-dichlorophenyl)-9-(4,4,5,5-tetramethyl-1,3,2-dioxaborolan-2-yl)-6,7-dihydro-5H-benzo[7]annulene-3-carboxylate as a white solid.

LC/MS (m/z, MH+): 473

### Intermediate 3: Methyl 8-(2,4-difluorophenyl)-9-(4,4,5,5-tetramethyl-1,3,2-dioxaborolan-2-yl)-6,7-dihydro-5H-benzo[7]annulene-3-carboxylate

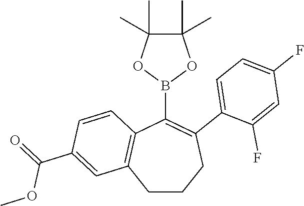 [SMILES: COC(=O)c1ccc2c(c1)CCCC(c1ccc(F)cc1F)=C2B1OC(C)(C)C(C)(C)O1]

### Step 1: Methyl 6-(2,4-difluorophenyl)-5-oxo-6,7,8,9-tetrahydro-5H-benzo[7]annulene-2-carboxylate

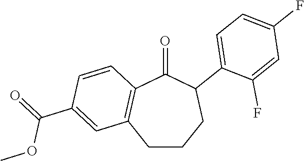 [SMILES: COC(=O)c1ccc2c(c1)CCCC(c1ccc(F)cc1F)C2=O]

Argon is bubbled for 10 minutes in a mixture of 1-bromo-2,4-difluoro-benzene (6.63 g, 34.37 mmol), methyl 5-oxo-6,7,8,9-tetrahydro-5H-benzo[7]annulene-2-carboxylate (5 g, 22.91 mmol), K₂CO₃ (12.67 g, 91.66 mmol) in toluene (40 ml). After addition of XANTPHOS (1.32 g, 2.29 mmol) and Pd₂(dba)₃ (1.05 g, 1.15 mmol), the reaction mixture was heated to reflux for 72 hours. After cooling to RT, water (40 ml) and DCM (40 ml) were added. After decantation, the aqueous phase was washed three times with 40 ml of DCM. The combined organic phases were dried over MgSO₄, filtered and concentrated under reduced pressure. The residue obtained was purified by flash chromatography, eluting with a gradient of Heptane/EtOAc from 100/00 to 90/10 to give 2.55 g (34%) of methyl 6-(2,4-difluorophenyl)-5-oxo-6,7,8,9-tetrahydro-5H-benzo[7]annulene-2-carboxylate. LC/MS (m/z, MH+): 331

### Step 2: Methyl 8-(2,4-difluorophenyl)-9-(((trifluoromethyl)sulfonyl)oxy)-6,7-dihydro-5H-benzo[7]annulene-3-carboxylate

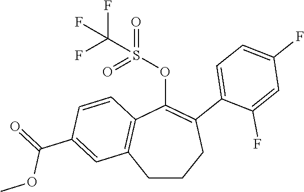 [SMILES: COC(=O)c1ccc2c(c1)CCCC(c1ccc(F)cc1F)=C2OS(=O)(=O)C(F)(F)F]

To a suspension of NaH (545 mg, 13.62 mmol, 60% purity in height) in Me-THF (22 ml) cooled at 5° C. was added DBU (277 mg, 0.27 ml, 1.82 mmol) followed by a solution of methyl 6-(2,4-difluorophenyl)-5-oxo-6,7,8,9-tetrahydrobenzo[7]annulene-2-carboxylate (3 g, 9.08 mmol) and N,N-bis(trifluoromethylsulfonyl)aniline (4.2 g, 11.81 mmol) in THF (25 ml). The cooling bath was removed to allow the temperature to warm up to RT. A mixture of acetic acid (0.4 ml) and water (32 ml) was dropwise added, followed by water (100 ml) and EtOAc (150 ml). After decantation, the organic phase was dried over MgSO₄, filtered and concentrated under reduced pressure The residue obtained was purified by flash chromatography, eluting with DCM/heptane 50/50 to give 2.77 g (66%) of methyl 8-(2,4-difluorophenyl)-9-(((trifluoromethyl)sulfonyl)oxy)-6,7-dihydro-5H-benzo[7]annulene-3-carboxylate.

LC/MS (m/z, MH+): 463

### Step 3: Methyl 8-(2,4-difluorophenyl)-9-(4,4,5,5-tetramethyl-1,3,2-dioxaborolan-2-yl)-6,7-dihydro-5H-benzo [7]annulene-3-carboxylate

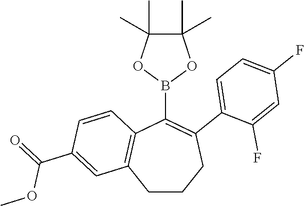 [SMILES: COC(=O)c1ccc2c(c1)CCCC(c1ccc(F)cc1F)=C2B1OC(C)(C)C(C)(C)O1]

Step 3 of Intermediate 3 was prepared following a similar procedure to that of Intermediate 1 from 8-(2,4-difluorophenyl)-9-(((trifluoromethyl)sulfonyl)oxy)-6,7-dihydro-5H-benzo[7]annulene-3-carboxylate to give 1.89 g (72%) of methyl 8-(2,4-difluorophenyl)-9-(4,4,5,5-tetramethyl-1,3,2-dioxaborolan-2-yl)-6,7-dihydro-5H-benzo[7]annulene-3-carboxylate.

LC/MS (m/z, MH+): 441

### Intermediate 4: Methyl 8-(2-methyl-4-fluorophenyl)-9-(4,4,5,5-tetramethyl-1,3,2-dioxaborolan-2-yl)-6,7-dihydro-5H-benzo[7]annulene-3-carboxylate

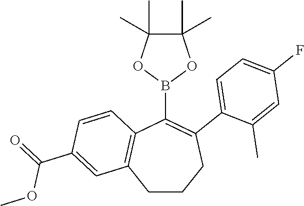 [SMILES: COC(=O)c1ccc2c(c1)CCCC(c1ccc(F)cc1C)=C2B1OC(C)(C)C(C)(C)O1]

### Step 1: Methyl 6-(2-methyl-4-fluorophenyl)-5-oxo-6,7,8,9-tetrahydro-5H-benzo[7]annulene-2-carboxylate

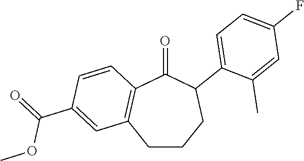 [SMILES: COC(=O)c1ccc2c(c1)CCCC(c1ccc(F)cc1C)C2=O]

Step 1 of Intermediate 4 was prepared following a similar procedure to that of step 1 of Intermediate 3 from 1-bromo-2-methyl-4-fluoro-benzene to give 700 mg (47%) of methyl 6-(2-methyl-4-fluorophenyl)-5-oxo-6,7,8,9-tetrahydro-5H-benzo[7]annulene-2-carboxylate.

LC/MS (m/z, MH+): 327

### Step 2: Methyl 8-(2-methyl-4-fluorophenyl)-9-(((trifluoromethyl)sulfonyl)oxy)-6,7-dihydro-5H-benzo [7]annulene-3-carboxylate

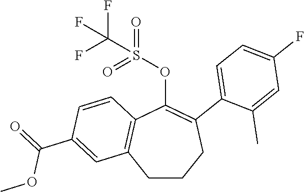 [SMILES: COC(=O)c1ccc2c(c1)CCCC(c1ccc(F)cc1C)=C2OS(=O)(=O)C(F)(F)F]

Step 2 of Intermediate 4 was prepared following a similar procedure to that of step 2 of Intermediate 3 from methyl 6-(2-methyl-4-fluorophenyl)-5-oxo-6,7,8,9-tetrahydrobenzo[7]annulene-2-carboxylate to give 7.86 g (82%) of methyl 8-(2-methyl-4-fluorophenyl)-9-(((trifluoromethyl)sulfonyl)oxy)-6,7-dihydro-5H-benzo[7]annulene-3-carboxylate.

LC/MS (m/z, MH+): 459

### Step 3: Methyl 8-(2-methyl-4-fluorophenyl)-9-(4,4,5,5-tetramethyl-1,3,2-dioxaborolan-2-yl)-6,7-dihydro-5H-benzo[7]annulene-3-carboxylate

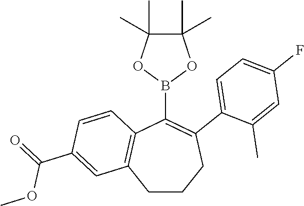 [SMILES: COC(=O)c1ccc2c(c1)CCCC(c1ccc(F)cc1C)=C2B1OC(C)(C)C(C)(C)O1]

Step 3 of Intermediate 4 was prepared following a similar procedure to that of Intermediate 1 from methyl 8-(2-methyl-4-fluorophenyl)-9-(((trifluoromethyl)sulfonyl)oxy)-6,7-dihydro-5H-benzo[7]annulene-3-carboxylate to give 3.93 g (43%) of methyl 8-(2-methyl-4-fluorophenyl)-9-(4,4,5,5-tetramethyl-1,3,2-dioxaborolan-2-yl)-6,7-dihydro-5H-benzo[7]annulene-3-carboxylate.

LC/MS (m/z, MH+): 437

### Intermediate 5: (4-Bromophenyl)-[1-(3-fluoropropyl)pyrrolidin-3-yl]methanol

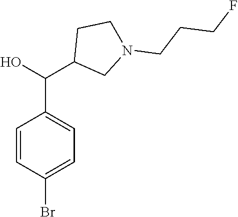 [SMILES: OC(c1ccc(Br)cc1)C1CCN(CCCF)C1]

### Step 1: (4-Bromophenyl)-pyrrolidin-3-yl-methanone, 2,2,2-trifluoroacetic acid

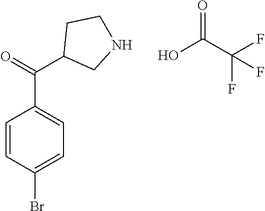 [SMILES: O=C(c1ccc(Br)cc1)C1CCNC1 ; O=C(O)C(F)(F)F]

To a solution of commercially available tert-butyl 3-(4-bromobenzoyl)pyrrolidine-1-carboxylate (20.3 g, 57.31 mmol) in DCM (100 ml) was added dropwise TFA (100 ml) and the reaction mixture was stirred at RT for 30 minutes. The reaction mixture was concentrated under reduced pressure and the residue obtained was triturated with Et₂O. The solid formed was filtered to give 17 g (81%) of (4-bromophenyl)-pyrrolidin-3-yl-methanone; 2,2,2-trifluoroacetic acid.

LC/MS (m/z, MH+): 254

### Step 2: (4-Bromophenyl)-[1-(3-fluoropropyl)pyrrolidin-3-yl]methanone

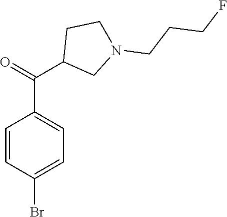 [SMILES: O=C(c1ccc(Br)cc1)C1CCN(CCCF)C1]

A mixture of (4-bromophenyl)-pyrrolidin-3-yl-methanone, 2,2,2-trifluoroacetic acid (9.45 g, 25.67 mmol), K₂CO₃ (10.64 g, 77 mmol), 1-fluoro-3-iodo-propane (5.07 g, 26.95 mmol) in MeCN (200 ml) was heated at 80° C. for 3 hours. The reaction mixture was quenched by addition of a saturated solution of NH₄Cl (200 ml) and extracted with EtOAc (3×200 ml). The organic phase was washed with brine (200 ml), dried over Na₂SO₄ filtered and concentrated under reduced pressure and the residue obtained was purified by flash chromatography, eluting with a gradient of DCM/MeOH: from 100/00 to 50/50 to 90/10 to give 6.34 g (78%) of (4-bromophenyl)-[1-(3-fluoropropyl)pyrrolidin-3-yl]methanone. LC/MS (m/z, MH+): 314

### Step 3: (4-Bromophenyl)-[1-(3-fluoropropyl)pyrrolidin-3-yl]methanol

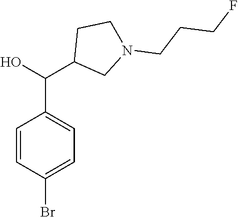 [SMILES: OC(c1ccc(Br)cc1)C1CCN(CCCF)C1]

To a mixture of (4-bromophenyl)-[1-(3-fluoropropyl)pyrrolidin-3-yl]methanone (6.31 g, 20.07 mmol) in MeOH (180 ml) was added NaBH₄ (2.28 g, 60.21 mmol) and the mixture was stirred at RT for 2 hours. A concentrated solution of NH₄Cl (100 ml) and EtOAc (300 ml) were added. After decantation, the organic phase was washed with brine (200 ml), dried over Na₂SO₄ filtered and concentrated under reduced pressure to give 6.69 g (crude) of (4-bromophenyl)-[1-(3-fluoropropyl)pyrrolidin-3-yl]methanol.

LC/MS (m/z, MH+): 316

### Intermediate 6: 3-[(4-Bromophenyl)methylene]-1-(3-fluoropropyl)pyrrolidine Cis isomer

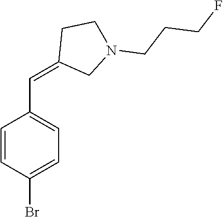 [SMILES: FCCCN1CC/C(=C/c2ccc(Br)cc2)C1]

To a mixture of (4-bromophenyl)-[1-(3-fluoropropyl)pyrrolidin-3-yl]methanol (Intermediate 5) (5.16 g, 16.33 mmol) and water (10 ml) was added dropwise at 0° C., sulfuric acid (35 ml). The cooling bath was removed, and the reaction mixture was stirred for 1 hour at RT. The reaction mixture was poured to a mixture of ice and water (500 ml). Powder NaHCO₃ was added to pH 9. EtOAc (300 ml) was added and the organic phase was dried over Na₂SO₄, filtered, concentrated under reduced pressure and the residue obtained was purified by flash chromatography eluting with a gradient of DCM/EtOAc: from 100/00 to 00/100 to give 4.13 g (85%) 3-[(4-bromophenyl)methylene]-1-(3-fluoropropyl)pyrrolidine as a mixture of Cis and Trans isomers.

The mixture of Cis and Trans isomers of 3-[(4-bromophenyl)methylene]-1-(3-fluoropropyl)pyrrolidine was separated by preparative SFC by using a IF 5×25 cm, 5 μm column (Daicel) and a CO₂ mobile phase with 30% of a 0.1% TEA in MeOH solution as co-solvent. The temperature was set at 40° C. and the back pressure regulator at 100 bars to give 1145 mg of Cis isomer (3Z)-3-[(4-bromophenyl)methylene]-1-(3-fluoropropyl)pyrrolidine and 2584 mg of Trans isomer (3E)-3-[(4-bromophenyl)methylene]-1-(3-fluoropropyl)pyrrolidine.

LC/MS (m/z, MH+): 298

### Intermediate 7: (4-Bromophenyl)-[1-(3,3-difluoropropyl)pyrrolidin-3-yl]methanol

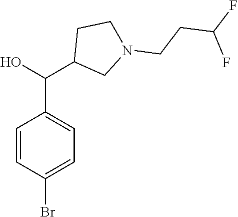 [SMILES: OC(c1ccc(Br)cc1)C1CCN(CCC(F)F)C1]

### Step 1: 3,3-Difluoropropyl 4-methylbenzenesulfonate

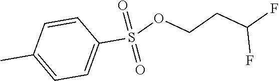 [SMILES: Cc1ccc(S(=O)(=O)OCCC(F)F)cc1]

To a solution of 3,3-difluoropropan-1-ol (500 mg, 5.20 mmol) in THF (10 ml) cooled at 0° C., was added NaH (229 mg, 5.72 mmol, 60 mass %). After 1 hour of stirring at 0° C., 4-methylbenzenesulfonyl chloride (1.03 g, 4.13 mmol) was added and the reaction mixture was stirred for 1 hour at RT. The reaction mixture was quenched by addition of a saturated solution of NH₄Cl (20 ml) and extracted with EtOAc (3×20 ml). The organic phase was washed with brine (20 ml), dried over Na₂SO₄ filtered and concentrated under reduced pressure and the residue obtained was purified by flash chromatography, eluting with DCM to give 1.03 g (79%) of 3,3-difluoropropyl 4-methylbenzenesulfonate.

LC/MS (m/z, MH+): 251

### Step 2: (4-Bromophenyl)-[1-(3,3-difluoropropyl)pyrrolidin-3-yl]methanone

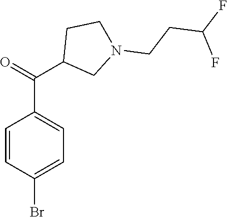 [SMILES: O=C(c1ccc(Br)cc1)C1CCN(CCC(F)F)C1]

Step 2 of Intermediate 7 was prepared following a similar procedure to that of step 2 of Intermediate 5 from (4-bromophenyl)-pyrrolidin-3-yl-methanone, 2,2,2-trifluoroacetic acid and 3,3-difluoropropyl 4-methylbenzenesulfonate to give 6.29 g (70%) of (4-bromophenyl)-[1-(3,3-difluoropropyl)pyrrolidin-3-yl]methanone.

LC/MS (m/z, MH+): 332

### Step 3: (4-Bromophenyl)-[1-(3,3-difluoropropyl)pyrrolidin-3-yl]methanol

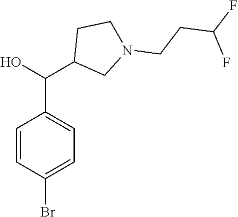 [SMILES: OC(c1ccc(Br)cc1)C1CCN(CCC(F)F)C1]

Step 3 of Intermediate 7 was prepared following a similar procedure to that of step 3 of Intermediate 5 from (4-bromophenyl)-[1-(3,3-difluoropropyl)pyrrolidin-3-yl]methanone to give 5.53 g (crude) of (4-bromophenyl)-[1-(3,3-difluoropropyl)pyrrolidin-3-yl]methanol.

LC/MS (m/z, MH+): 334

### Intermediate 8: 3-[(4-Bromophenyl)methylene]-1-(3,3-difluoropropyl)pyrrolidine Cis isomer

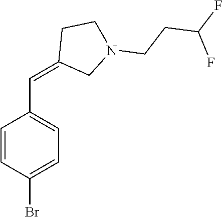 [SMILES: FC(F)CCN1CC/C(=C/c2ccc(Br)cc2)C1]

Intermediate 8 was prepared following a similar procedure to that of Intermediate 6 from (4-bromophenyl)-[1-(3,3-difluoropropyl)pyrrolidin-3-yl]methanol (Intermediate 7) to give 2.71 g (64%) 3-[(4-bromophenyl)methylene]-1-(3,3-difluoropropyl)pyrrolidine as a mixture of Cis and Trans isomers.

The mixture of Cis and Trans isomers of 3-[(4-bromophenyl)methylene]-1-(3-fluoropropyl)pyrrolidine was separated by preparative SFC by using a IG 5×25 cm, 5 μm column (Daicel) and a CO₂ mobile phase with 30% of a 0.1% TEA in MeOH solution as cosolvent. The temperature was set at 40° C. and the back pressure regulator at 100 bars to give 158 mg of Cis isomer (3Z)-3-[(4-bromophenyl)methylene]-1-(3,3-difluoropropyl)pyrrolidine and 299 mg of Trans isomer (3E)-3-[(4-bromophenyl)methylene]-1-(3,3-difluoropropyl)pyrrolidine.

LC/MS (m/z, MH+): 316

### Intermediate 9: Methyl (Z)-8-bromo-9-(4-((1-(3-fluoropropyl)pyrrolidin-3-ylidene)methyl)phenyl)-6,7-dihydro-5H-benzo[7]annulene-3-carboxylate

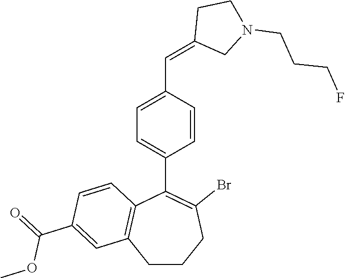 [SMILES: COC(=O)c1ccc2c(c1)CCCC(Br)=C2c1ccc(/C=C2/CCN(CCCF)C2)cc1]

### Step 1: Methyl 9-(4-((1-(3-fluoropropyl)pyrrolidin-3-yl)(hydroxy)methyl)phenyl)-6,7-dihydro-5H-benzo [7]annulene-3-carboxylate

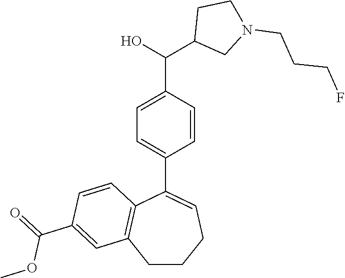 [SMILES: COC(=O)c1ccc2c(c1)CCCC=C2c1ccc(C(O)C2CCN(CCCF)C2)cc1]

A mixture of methyl 9-(4,4,5,5-tetramethyl-1,3,2-dioxaborolan-2-yl)-6,7-dihydro-5H-benzo[7]annulene-3-carboxylate (Intermediate 1) (3.42, 10.44 mmol), (4-bromophenyl)-[1-(3-fluoropropyl)pyrrolidin-3-yl]methanol (Intermediate 5) (3 g, 9.49 mmol) Cs₂CO₃ (8.04 g, 24.67 mmol), Pd(dppf)C₁₂ complex with DCM (466 mg, 0.57 mmol), in dioxane (24 ml) and water (6 ml) was heated to reflux for 1 hour. The reaction mixture was quenched by addition of a saturated solution of NH₄Cl(50 ml) and extracted with EtOAc (3×50 ml). The organic phase was washed with brine (50 ml), dried over Na₂SO₄ filtered and concentrated under reduced pressure and the residue obtained by flash chromatography, eluting with a gradient of DCM/MeOH: from 100/00 to 80/20 to give 2.59 g (62%) of methyl 9-(4-((1-(3-fluoropropyl)pyrrolidin-3-yl)(hydroxy)methyl)phenyl)-6,7-dihydro-5H-benzo[7]annulene-3-carboxylate.

LC/MS (m/z, MH+): 438

### Step 2: Methyl 8-bromo-9-(4-((1-(3-fluoropropyl)pyrrolidin-3-yl)(hydroxy)methyl)phenyl)-6,7-dihydro-5H-benzo[7]annulene-3-carboxylate

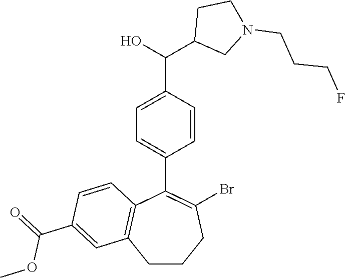 [SMILES: COC(=O)c1ccc2c(c1)CCCC(Br)=C2c1ccc(C(O)C2CCN(CCCF)C2)cc1]

To a mixture of methyl 9-(4-((1-(3-fluoropropyl)pyrrolidin-3-yl)(hydroxy)methyl)phenyl)-6,7-dihydro-5H-benzo[7]annulene-3-carboxylate (2.60 g, 5.94 mmol) in DCM (100 ml) was added pyridinium tribromide (2.66 g, 8.31 mmol). The reaction mixture was stirred for 18 hours at RT. The reaction was quenched with concentrated solution of NaHCO₃ (100 ml) and DCM (150 ml). The organic phase was dried over Na₂SO₄, filtered, concentrated under reduced pressure and the residue obtained was purified by flash chromatography eluting with a gradient of DCM/MeOH: from 100/00 to 80/20 to give 1.53 g (50%) of methyl 8-bromo-9-(4-((1-(3-fluoropropyl)pyrrolidin-3-yl)(hydroxy)methyl)phenyl)-6,7-dihydro-5H-benzo [7]annulene-3-carboxylate.

LC/MS (m/z, MH+): 516

### Step 3: Methyl (Z)-8-bromo-9-(4-((1-(3-fluoropropyl)pyrrolidin-3-ylidene)methyl)phenyl)-6,7-dihydro-5H-benzo[7]annulene-3-carboxylate

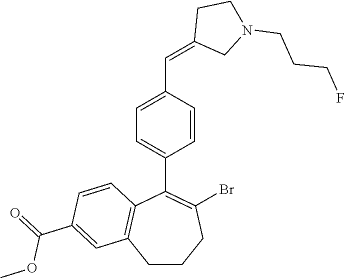 [SMILES: COC(=O)c1ccc2c(c1)CCCC(Br)=C2c1ccc(/C=C2/CCN(CCCF)C2)cc1]

To a mixture of methyl 8-bromo-9-(4-((1-(3-fluoropropyl)pyrrolidin-3-yl)(hydroxy)methyl)phenyl)-6,7-dihydro-5H-benzo[7]annulene-3-carboxylate (1.53 g, 2.97 mmol) and water (2 ml) was added dropwise at 0° C., sulfuric acid (7 ml). the cooling bath was removed and the reaction mixture was stirred for 30 minutes at RT. The reaction mixture was poured to a mixture of ice and water (150 ml). Powder NaHCO₃ was added to pH 9. EtOAc (100 ml) was added and the organic phase was dried over Na₂SO₄, filtered, concentrated under reduced pressure and the residue obtained was purified by flash chromatography eluting with a gradient of DCM/EtOAc: from 100/00 to 00/100 to give 1.04 g (69%) of methyl 8-bromo-9-(4-((1-(3-fluoropropyl)pyrrolidin-3-ylidene)methyl)phenyl)-6,7-dihydro-5H-benzo[7]annulene-3-carboxylate as a mixture of Cis and Trans isomers. The mixture of Cis and Trans isomers of methyl 8-bromo-9-(4-((1-(3-fluoropropyl)pyrrolidin-3-ylidene)methyl)phenyl)-6,7-dihydro-5H-benzo[7]annulene-3-carboxylate was separated by preparative SFC by using a IB-N5 5×25 cm, 5 μm column (Daicel) and a CO₂ mobile phase with 23% of a 0.1% TEA in MeOH solution as cosolvent. The temperature was set at 40° C. and the back pressure regulator at 100 bars to give 170 mg of Cis isomer methyl (Z)-8-bromo-9-(4-((1-(3-fluoropropyl)pyrrolidin-3-ylidene)methyl)phenyl)-6,7-dihydro-5H-benzo[7]annulene-3-carboxylate and 388 mg of Trans isomer methyl (E)-8-bromo-9-(4-((1-(3-fluoropropyl)pyrrolidin-3-ylidene)methyl)phenyl)-6,7-dihydro-5H-benzo[7]annulene-3-carboxylate.

LC/MS (m/z, MH+): 498

### Intermediate 10: Methyl (Z)-8-bromo-9-(4-((1-(3,3-difluoropropyl)pyrrolidin-3-ylidene)methyl)phenyl)-6,7-dihydro-5H-benzo[7]annulene-3-carboxylate

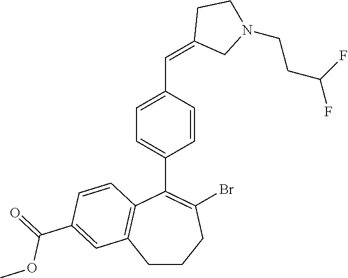 [SMILES: COC(=O)c1ccc2c(c1)CCCC(Br)=C2c1ccc(/C=C2/CCN(CCC(F)F)C2)cc1]

### Step 1: Methyl 9-(4-((1-(3,3-difluoropropyl)pyrrolidin-3-yl)(hydroxy)methyl)phenyl)-6,7-dihydro-5H-benzo [7]annulene-3-carboxylate

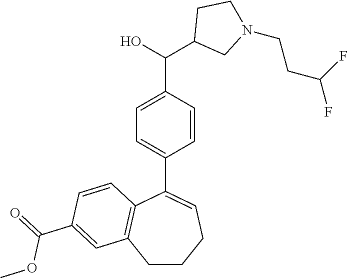 [SMILES: COC(=O)c1ccc2c(c1)CCCC=C2c1ccc(C(O)C2CCN(CCC(F)F)C2)cc1]

Step 1 of Intermediate 10 was prepared following a similar procedure to that of step 1 of Intermediate 9 from methyl 9-(4,4,5,5-tetramethyl-1,3,2-dioxaborolan-2-yl)-6,7-dihydro-5H-benzo[7]annulene-3-carboxylate (Intermediate 1) and (4-bromophenyl)-[1-(3,3-difluoropropyl)pyrrolidin-3-yl]methanol (Intermediate 7) to give 5.98 g (80%) of methyl 9-(4-((1-(3,3-difluoropropyl)pyrrolidin-3-yl)(hydroxy)methyl)phenyl)-6,7-dihydro-5H-benzo[7]annulene-3-carboxylate.

LC/MS (m/z, MH+): 456

### Step 2: Methyl 8-bromo-9-(4-((1-(3,3-difluoropropyl)pyrrolidin-3-yl)(hydroxy)methyl)phenyl)-6,7-dihydro-5H-benzo[7]annulene-3-carboxylate

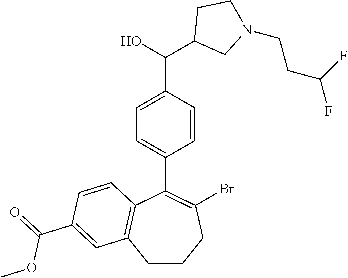 [SMILES: COC(=O)c1ccc2c(c1)CCCC(Br)=C2c1ccc(C(O)C2CCN(CCC(F)F)C2)cc1]

Step 2 of Intermediate 10 was prepared following a similar procedure to that of step 2 of Intermediate 9 from methyl 9-(4-((1-(3,3-difluoropropyl)pyrrolidin-3-yl)(hydroxy)methyl)phenyl)-6,7-dihydro-5H-benzo[7]annulene-3-carboxylate to give 3.93 g (56%) of methyl 8-bromo-9-(4-((1-(3,3-difluoropropyl)pyrrolidin-3-yl)(hydroxy)methyl)phenyl)-6,7-dihydro-5H-benzo[7]annulene-3-carboxylate.

LC/MS (m/z, MH+): 534

### Step 3: Methyl (Z)-8-bromo-9-(4-((1-(3,3-difluoropropyl)pyrrolidin-3-ylidene)methyl)phenyl)-6,7-dihydro-5H-benzo[7]annulene-3-carboxylate

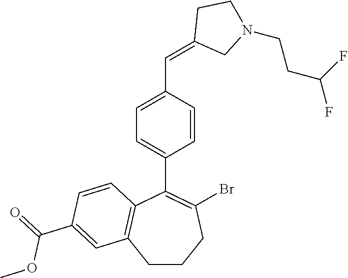 [SMILES: COC(=O)c1ccc2c(c1)CCCC(Br)=C2c1ccc(/C=C2/CCN(CCC(F)F)C2)cc1]

Step 3 of Intermediate 10 was prepared following a similar procedure to that of step 3 of Intermediate 9 from methyl 8-bromo-9-(4-((1-(3,3-difluoropropyl)pyrrolidin-3-yl)(hydroxy)methyl)phenyl)-6,7-dihydro-5H-benzo[7]annulene-3-carboxylate to give 2.46 g (65%) of methyl 8-bromo-9-(4-((1-(3,3-difluoropropyl)pyrrolidin-3-ylidene)methyl)phenyl)-6,7-dihydro-5H-benzo[7]annulene-3-carboxylate as a mixture of Cis and Trans isomers.

The mixture of Cis and Trans isomers of methyl 8-bromo-9-(4-((1-(3,3-difluoropropyl)pyrrolidin-3-ylidene)methyl)phenyl)-6,7-dihydro-5H-benzo[7]annulene-3-carboxylate was separated by chiral chromatography: Chiralpak AD 20 μM (250×4.6 mm) eluting with heptane/EtOH/TEA:50/50/0.1 to give 562 mg of Cis isomer methyl (Z)-8-bromo-9-(4-((1-(3,3-difluoropropyl)pyrrolidin-3-ylidene)methyl)phenyl)-6,7-dihydro-5H-benzo[7]annulene-3-carboxylate and 986 mg of Trans isomer methyl (E)-8-bromo-9-(4-((1-(3,3-difluoropropyl)pyrrolidin-3-ylidene)methyl)phenyl)-6,7-dihydro-5H-benzo[7]annulene-3-carboxylate.

LC/MS (m/z, MH+): 516

### Intermediate 11: 2-(8-(2-Chlorophenyl)-6,7-dihydro-5H-benzo[7]annulen-9-yl)-4,4,5,5-tetramethyl-1,3,2-dioxaborolane

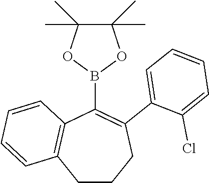 [SMILES: CC1(C)OB(C2=C(c3ccccc3Cl)CCCc3ccccc32)OC1(C)C]

### Step 1: 6-(2-Chlorophenyl)-6,7,8,9-tetrahydro-5H-benzo[7]annulen-5-one

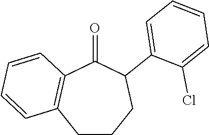 [SMILES: O=C1c2ccccc2CCCC1c1ccccc1Cl]

Step 1 of Intermediate 11 was prepared following a similar procedure to that of Step 1 of Intermediate 3 from 1-bromo-2-chloro-benzene and 6,7,8,9-tetrahydrobenzo[7]annulen-5-one to give 580 mg (16%) of 6-(2-chlorophenyl)-6,7,8,9-tetrahydro-5H-benzo[7]annulen-5-one.

LC/MS (m/z, MH+): 271

### Step 2: 8-(2-Chlorophenyl)-6,7-dihydro-5H-benzo[7]annulen-9-yl trifluoromethanesulfonate

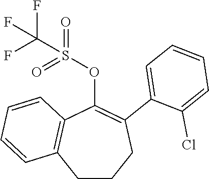 [SMILES: O=S(=O)(OC1=C(c2ccccc2Cl)CCCc2ccccc21)C(F)(F)F]

Step 2 of Intermediate 11 was prepared following a similar procedure to that of Step 2 of Intermediate 3 from 6-(2-chlorophenyl)-6,7,8,9-tetrahydro-5H-benzo[7]annulen-5-one to give 608 mg (71%) of 8-(2-chlorophenyl)-6,7-dihydro-5H-benzo[7]annulen-9-yl trifluoromethanesulfonate.

LC/MS (m/z, MH+): 403

### Step 3: 2-(8-(2-Chlorophenyl)-6,7-dihydro-5H-benzo[7]annulen-9-yl)-4,4,5,5-tetramethyl-1,3,2-dioxaborolane

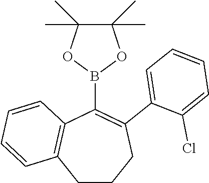 [SMILES: CC1(C)OB(C2=C(c3ccccc3Cl)CCCc3ccccc32)OC1(C)C]

Step 3 of Intermediate 11 was prepared following a similar procedure to that of Intermediate 1 from 8-(2-chlorophenyl)-6,7-dihydro-5H-benzo[7]annulen-9-yl trifluoromethanesulfonate to give 430 mg (84%) of 2-(8-(2-chlorophenyl)-6,7-dihydro-5H-benzo[7]annulen-9-yl)-4,4,5,5-tetramethyl-1,3,2-dioxaborolane.

LC/MS (m/z, MH+): 381

### Intermediate 12: Methyl 4-(2,4-dichlorophenyl)-5-(4,4,5,5-tetramethyl-1,3,2-dioxaborolan-2-yl)-2,3-dihydrobenzo[b]oxepine-8-carboxylate

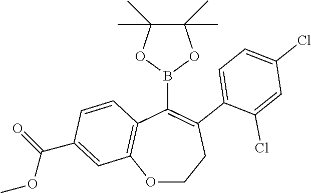 [SMILES: COC(=O)c1ccc2c(c1)OCCC(c1ccc(Cl)cc1Cl)=C2B1OC(C)(C)C(C)(C)O1]

### Step 1: 5-Oxo-2,3,4,5-tetrahydrobenzo[b]oxepine-8-yl trifluoromethanesulfonate

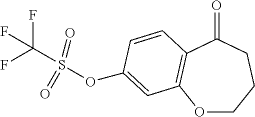 [SMILES: O=C1CCCOc2cc(OS(=O)(=O)C(F)(F)F)ccc21]

To a mixture of 8-hydroxy-3,4-dihydrobenzo [b]oxepine-5(2H)-one (4.2 g, 23.57 mmol) (prepared according to WO2018091153) and pyridine (2.82 g, 2.89 ml, 35.36 mmol) in DCM (120 ml) cooled at −20° C. was dropwise added trifluoromethanesulfonic anhydride (8.14 g, 6 ml, 28.28 mmol). The reaction mixture was stirred at 0° C. for 30 minutes. Water (50 ml) was added. The organic phase was separated and washed with an aqueous saturated solution of NaHCO₃ (50 ml). The organic phase was dried over MgSO₄, filtered, and concentrated under reduced pressure to give 7.40 g (crude) of 5-oxo-2,3,4,5-tetrahydrobenzo[b]oxepine-8-yl trifluoromethanesulfonate.

LC/MS (m/z, MH+): 311

### Step 2: Methyl 5-oxo-2,3,4,5-tetrahydrobenzo[b]oxepine-8-carboxylate

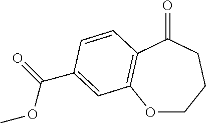 [SMILES: COC(=O)c1ccc2c(c1)OCCCC2=O]

To a solution of compound 5-oxo-2,3,4,5-tetrahydrobenzo[b]oxepine-8-yl trifluoromethanesulfonate (7.4 g, 23.85 mmol) in DMF (30 ml) and MeOH (15 ml) was added DIEA (3.15 g, 4.16 ml, 23.85 mmol) and Pd(dppf)Cl₂ complex with DCM (1.10 g, 1.43 mmol), the suspension was degassed and purged three times with CO. The mixture was stirred under CO (5 bars) at 75° C. for 2 hours. The reaction was filtered through celite. The filtrate was diluted with water (400 ml) and extracted three times with EtOAc (300 ml). The combined organic phases were dried over Na₂SO₄, filtered, concentrated under reduced pressure, and the residue obtained was purified by flash chromatography eluting with a gradient of heptane/ethyl acetate: from 85/15 to 80/20 to give 4.6 g (88%) methyl 5-oxo-2,3,4,5-tetrahydrobenzo[b]oxepine-8-carboxylate.

LC/MS (m/z, MH+): 221

### Step 3: Methyl 4-(2,4-dichlorophenyl)-5-oxo-2,3,4,5-tetrahydrobenzo[b]oxepine-8-carboxylate

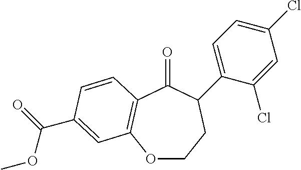 [SMILES: COC(=O)c1ccc2c(c1)OCCC(c1ccc(Cl)cc1Cl)C2=O]

Step 3 of Intermediate 12 was prepared following a similar procedure to that of step 1 of Intermediate 3 from methyl 5-oxo-2,3,4,5-tetrahydrobenzo[b]oxepine-8-carboxylate to give 1.13 g (67%) of methyl 4-(2,4-dichlorophenyl)-5-oxo-2,3,4,5-tetrahydrobenzo[b]oxepine-8-carboxylate.

LC/MS (m/z, MH+): 365

### Step 4: Methyl 4-(2,4-dichlorophenyl)-5-(((trifluoromethyl)sulfonyl)oxy)-2,3-dihydrobenzo[b]oxepine-8-carboxylate

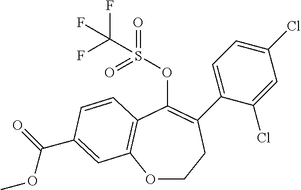 [SMILES: COC(=O)c1ccc2c(c1)OCCC(c1ccc(Cl)cc1Cl)=C2OS(=O)(=O)C(F)(F)F]

Step 4 of Intermediate 12 was prepared following a similar procedure to that of step 2 of Intermediate 3 from methyl 4-(2,4-dichlorophenyl)-5-oxo-2,3,4,5-tetrahydrobenzo[b]oxepine-8-carboxylate to give 0.48 g (31%) of methyl 4-(2,4-dichlorophenyl)-5-(((trifluoromethyl)sulfonyl)oxy)-2,3-dihydrobenzo[b]oxepine-8-carboxylate.

LC/MS (m/z, MH+): 496

### Step 5: Methyl 4-(2,4-dichlorophenyl)-5-(4,4,5,5-tetramethyl-1,3,2-dioxaborolan-2-yl)-2,3-dihydrobenzo[b]oxepine-8-carboxylate

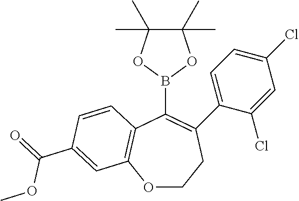 [SMILES: COC(=O)c1ccc2c(c1)OCCC(c1ccc(Cl)cc1Cl)=C2B1OC(C)(C)C(C)(C)O1]

Step 5 of Intermediate 12 was prepared following a similar procedure to that of Intermediate 1 from methyl 4-(2,4-dichlorophenyl)-5-(((trifluoromethyl)sulfonyl)oxy)-2,3-dihydrobenzo[b]oxepine-8-carboxylate to give 260 mg (61%) of methyl 4-(2,4-dichlorophenyl)-5-(4,4,5,5-tetramethyl-1,3,2-dioxaborolan-2-yl)-2,3-dihydrobenzo[b]oxepine-8-carboxylate.

LC/MS (m/z, MH+): 271

### Intermediate 13: 8-(2,4-Dichlorophenyl)-9-(((trifluoromethyl)sulfonyl)oxy)-6,7-dihydro-5H-benzo[7]annulen-3-yl pivalate

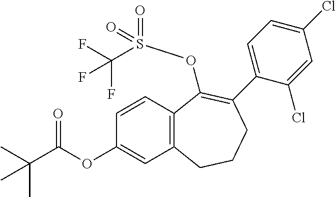 [SMILES: CC(C)(C)C(=O)Oc1ccc2c(c1)CCCC(c1ccc(Cl)cc1Cl)=C2OS(=O)(=O)C(F)(F)F]

### Step 1: 6-(2,4-Dichlorophenyl)-2-methoxy-6,7,8,9-tetrahydro-5H-benzo[7]annulen-5-one

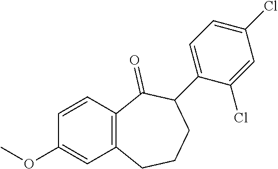 [SMILES: COc1ccc2c(c1)CCCC(c1ccc(Cl)cc1Cl)C2=O]

A mixture of 1-bromo-2,4-dichloro-benzene (890 mg, 3.94 mmol), 2-methoxy-6,7,8,9-tetrahydro-5H-benzo[7]annulen-5-one (500 mg; 2.63 mmol), XANTPHOS (0.304 g, 0.53 mmol) and Pd₂(dba)₃ (0.3 g, 0.53 mmol) in xylene (10 ml) was heated to reflux for 16 hours. After cooling to RT, water (50 ml) and Et₂O (50 ml) were added. The organic phase was dried over MgSO₄, filtered and concentrated under reduced pressure. The residue obtained was purified by flash chromatography, eluting with a gradient of cyclohexane/EtOAc from 100/00 to 95/05 to give 470 mg (42%) of 6-(2,4-dichlorophenyl)-2-methoxy-6,7,8,9-tetrahydro-5H-benzo [7]annulen-5-one.

LC/MS (m/z, MH+): 335

### Step 2: 6-(2,4-Dichlorophenyl)-2-hydroxy-6,7,8,9-tetrahydro-5H-benzo[7]annulen-5-one

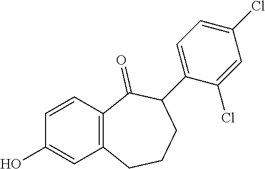 [SMILES: O=C1c2ccc(O)cc2CCCC1c1ccc(Cl)cc1Cl]

A mixture of 6-(2,4-dichlorophenyl)-2-methoxy-6,7,8,9-tetrahydro-5H-benzo[7]annulen-5-one (470 mg, 1.23 mmol) and aluminium chloride (395 mg, 2.96 mmol) in toluene (8 ml) was heated at 90° C. for 2 hours. After cooling to room temperature, water (20 ml) and EtOAc (50 ml) were added. The organic phase was dried over MgSO₄, filtered and concentrated under reduced pressure. The residue obtained was purified by flash chromatography, eluting with a gradient of cyclohexane/EtOAc from 100/00 to 80/20 to give 165 mg (42%) of 6-(2,4-dichlorophenyl)-2-hydroxy-6,7,8,9-tetrahydro-5H-benzo[7]annulen-5-one.

LC/MS (m/z, MH+): 321

### Step 3: 6-(2,4-Dichlorophenyl)-5-oxo-6,7,8,9-tetrahydro-5H-benzo[7]annulen-2-yl pivalate

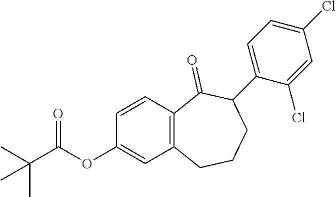 [SMILES: CC(C)(C)C(=O)Oc1ccc2c(c1)CCCC(c1ccc(Cl)cc1Cl)C2=O]

A mixture of 6-(2,4-dichlorophenyl)-2-hydroxy-6,7,8,9-tetrahydro-5H-benzo[7]annulen-5-one (163 mg, 0.51 mmol), K₂CO₃ (77 mg, 0.56 mmol) and pivaloyl chloride (67 mg, 0.56 mmol) in acetone (5 ml) was heated at 40° C. for 1 hour. After cooling to RT, the reaction mixture was filtered. The filtrate was concentrated under reduced pressure and the residue obtained was purified by flash chromatography with a gradient of cyclohexane/EtOAc from 100/00 to 90/10 to give 205 mg (96%) of 6-(2,4-dichlorophenyl)-5-oxo-6,7,8,9-tetrahydro-5H-benzo[7]annulen-2-yl pivalate.

LC/MS (m/z, MH+): 405

### Step 4: 8-(2,4-Dichlorophenyl)-9-(((trifluoromethyl)sulfonyl)oxy)-6,7-dihydro-5H-benzo[7]annulen-3-yl pivalate

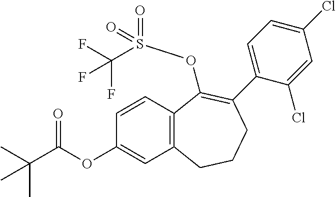 [SMILES: CC(C)(C)C(=O)Oc1ccc2c(c1)CCCC(c1ccc(Cl)cc1Cl)=C2OS(=O)(=O)C(F)(F)F]

To a solution of 6-(2,4-dichlorophenyl)-5-oxo-6,7,8,9-tetrahydro-5H-benzo[7]annulen-2-yl pivalate (197 mg, 0.48 mmol) in THF (5 ml) was added KHMDS (1 M, 0.58 ml, 0.58 mmol) at −50° C. under Ar atmosphere. The mixture was stirred for 30 min and 1,1,1-trifluoro-N-phenyl-N-(trifluoromethylsulfonyl)methanesulfonamide (173 mg, 0.49 mmol) was added to the resulting mixture. The reaction mixture was slowly warmed up to 20° C. and stirred for 90 minutes. The reaction mixture was quenched with saturated aqueous citric acid solution, then diluted with water and extracted twice with Et₂O. The combined organic layers were dried over anhydrous Na₂SO₄, filtered, and concentrated under reduced pressure. The residue was purified by flash chromatography with a gradient of cyclohexane/EtOAc from 100/00 to 90/10 to give 160 mg (61%) of 8-(2,4-dichlorophenyl)-9-(((trifluoromethyl)sulfonyl)oxy)-6,7-dihydro-5H-benzo[7]annulen-3-yl pivalate.

LC/MS (m/z, MH+): 537

### Intermediate 14: (3Z)-1-(3-fluoropropyl)-3-[[4-(4,4,5,5-tetramethyl-1,3,2-dioxaborolan-2-yl)phenyl]methylene]pyrrolidine

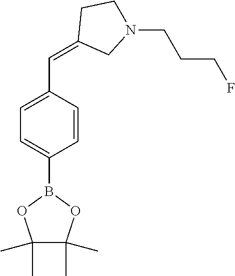 [SMILES: CC1(C)OB(c2ccc(/C=C3/CCN(CCCF)C3)cc2)OC1(C)C]

A mixture of (3Z)-3-[(4-bromophenyl)methylene]-1-(3-fluoropropyl)pyrrolidine (Intermediate 6) (200 mg, 0.67 mmol), in dioxane (4 ml), Pd(PPh₃)₂Cl₂ (38 mg, 0.07 mmol), bis(pinacolato)diboron (221 mg, 0.87 mmol) and AcOK (164 mg, 1.68 mmol) was heated at reflux for 2 hours. After cooling, the reaction mixture was filtered on dicalite. The filtrate was concentrated under reduced pressure and the residue was purified by flash chromatography, eluting with a gradient of DCM/EtOAc/EtOH: from 100/00/00 to 75/20/05 to give 160 mg (69%) of (3Z)-1-(3-fluoropropyl)-3-[[4-(4,4,5,5-tetramethyl-1,3,2-dioxaborolan-2-yl)phenyl]methylene]pyrrolidine.

LC/MS (m/z, MH+): 346

### Intermediate 15: (3Z)-1-(3,3-difluoropropyl)-3-[[4-(4,4,5,5-tetramethyl-1,3,2-dioxaborolan-2-yl)phenyl]methylene]pyrrolidine

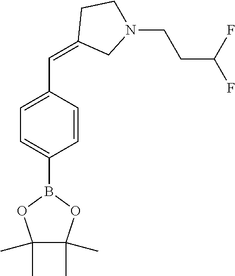 [SMILES: CC1(C)OB(c2ccc(/C=C3/CCN(CCC(F)F)C3)cc2)OC1(C)C]

Intermediate 15 was prepared following a similar procedure to that of Intermediate 14 from (3Z)-3-[(4-bromophenyl)methylene]-1-(3,3-difluoropropyl)pyrrolidine (Intermediate 8) to give 114.8 mg (87%) of (3Z)-1-(3,3-difluoropropyl)-3-[[4-(4,4,5,5-tetramethyl-1,3,2-dioxaborolan-2-yl)phenyl]methylene]pyrrolidine.

LC/MS (m/z, MH+): 364

### Intermediate 16: Methyl 6-(2,4-dichlorophenyl)-5-(trifluoromethylsulfonyloxy)-7,8-dihydronaphthalene-2-carboxylate

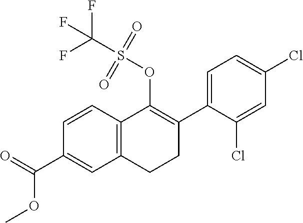 [SMILES: COC(=O)c1ccc2c(c1)CCC(c1ccc(Cl)cc1Cl)=C2OS(=O)(=O)C(F)(F)F]

### Step 1: Methyl 2-(2,4-dichlorophenyl)-1-oxo-tetralin-6-carboxylate

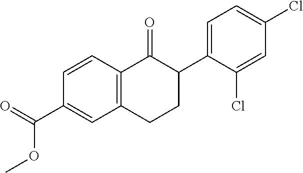 [SMILES: COC(=O)c1ccc2c(c1)CCC(c1ccc(Cl)cc1Cl)C2=O]

Step 1 of Intermediate 16 was prepared following a similar procedure to that of step 1 of Intermediate 3 from methyl 1-oxotetralin-6-carboxylate to give 459 mg (19%) of methyl 4-(2,4-dichlorophenyl)-5-oxo-2,3,4,5-tetrahydrobenzo[b]oxepine-8-carboxylate. LC/MS (m/z, MH+): 349

### Step 2: Methyl 6-(2,4-dichlorophenyl)-5-(trifluoromethylsulfonyloxy)-7,8-dihydronaphthalene-2-carboxylate

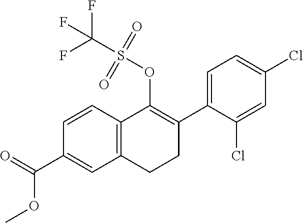 [SMILES: COC(=O)c1ccc2c(c1)CCC(c1ccc(Cl)cc1Cl)=C2OS(=O)(=O)C(F)(F)F]

Step 2 of Intermediate 16 was prepared following a similar procedure to that of step 2 of Intermediate 3 from methyl 2-(2,4-dichlorophenyl)-1-oxo-tetralin-6-carboxylate to give 0.48 g (31%) of methyl 6-(2,4-dichlorophenyl)-5-(trifluoromethylsulfonyloxy)-7,8-dihydronaphthalene-2-carboxylate.

LC/MS (m/z, MH+): 481

### Intermediate 17: (Z)-3-(4-Bromo-2-fluorobenzylidene)-1-(3-fluoropropyl)pyrrolidine

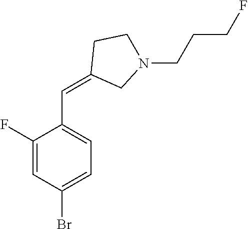 [SMILES: FCCCN1CC/C(=C/c2ccc(Br)cc2F)C1]

### Step 1: Tert-butyl 3-(4-bromo-2-fluorobenzoyl)pyrrolidine-1-carboxylate

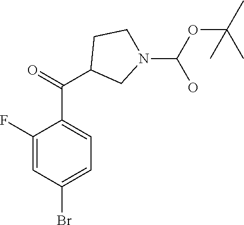 [SMILES: CC(C)(C)OC(O)N1CCC(C(=O)c2ccc(Br)cc2F)C1]

To a solution of 1,4-dibromo-2-fluoro-benzene (11.3 g, 44.52 mmol) in THF (60 ml) cooled at −78° C., was dropwise added n-BuLi 2.5 M (18 ml, 44.52 mmol). After stirring for 45 minutes at −78° C., a solution of tert-butyl 3-[methoxy(methyl)carbamoyl]pyrrolidine-1-carboxylate (10 g, 38.71 mmol) in THF (60 ml) was dropwise added. The cooling bath was removed and the reaction mixture was stirred for 18 hours at RT. The reaction mixture was poured into a mixture of saturated NH₄Cl aqueous solution (200 ml) and EtOAc (300 ml). After decantation, the organic layers were dried over anhydrous Na₂SO₄, filtered, and concentrated under reduced pressure. The residue was purified by flash chromatography with a gradient of DCM/EtOAc from 100/0 to 80/20 to give 2.27 g (13%) of tert-butyl 3-(4-bromo-2-fluorobenzoyl)pyrrolidine-1-carboxylate.

LC/MS (m/z, MH+): 372

### Step 2: (4-Bromo-2-fluorophenyl)(pyrrolidin-3-yl)methanone, 2,2,2-trifluoroacetic acid

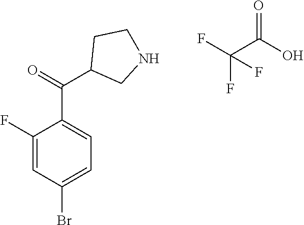 [SMILES: O=C(c1ccc(Br)cc1F)C1CCNC1 ; O=C(O)C(F)(F)F]

Step 2 of Intermediate 17 was prepared following a similar procedure to that of step 1 of Intermediate 5 from tert-butyl 3-(4-bromo-2-fluorobenzoyl)pyrrolidine-1-carboxylate to give 1.96 g (84%) of (4-bromo-2-fluorophenyl)(pyrrolidin-3-yl)methanone, 2,2,2-trifluoroacetic acid.

LC/MS (m/z, MH+): 272

### Step 3: (4-Bromo-2-fluorophenyl)(1-(3-fluoropropyl)pyrrolidin-3-yl)methanone

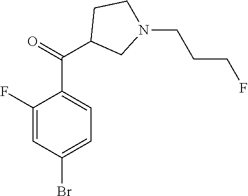 [SMILES: O=C(c1ccc(Br)cc1F)C1CCN(CCCF)C1]

Step 3 of Intermediate 17 was prepared following a similar procedure to that of step 2 of Intermediate 5 from (4-bromo-2-fluorophenyl)(pyrrolidin-3-yl)methanone, 2,2,2-trifluoroacetic acid to give 1.13 g (67%) of (4-bromo-2-fluorophenyl)(1-(3-fluoropropyl)pyrrolidin-3-yl)methanone.

LC/MS (m/z, MH+): 332

### Step 4: (4-Bromo-2-fluorophenyl)(1-(3-fluoropropyl)pyrrolidin-3-yl)methanol

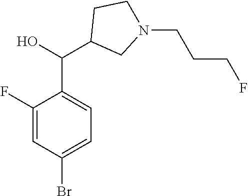 [SMILES: OC(c1ccc(Br)cc1F)C1CCN(CCCF)C1]

Step 4 of Intermediate 17 was prepared following a similar procedure to that of step 3 of Intermediate 5 from (4-bromo-2-fluorophenyl)(1-(3-fluoropropyl)pyrrolidin-3-yl)methanone to give 2.1 g (97%) of (4-bromo-2-fluorophenyl)(1-(3-fluoropropyl)pyrrolidin-3-yl)methanol.

LC/MS (m/z, MH+): 334

### Step 5: (Z)-3-(4-Bromo-2-fluorobenzylidene)-1-(3-fluoropropyl)pyrrolidine

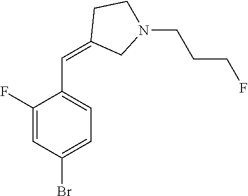 [SMILES: FCCCN1CC/C(=C/c2ccc(Br)cc2F)C1]

Step 5 of Intermediate 17 was prepared following a similar procedure to that of Intermediate 6 from (4-bromo-2-fluorophenyl)(1-(3-fluoropropyl)pyrrolidin-3-yl)methanol to give 925 mg of 3-(4-bromo-2-fluorobenzylidene)-1-(3-fluoropropyl)pyrrolidine as a mixture of Cis and Trans isomers.

The mixture of Cis and Trans isomers of 3-(4-bromo-2-fluorobenzylidene)-1-(3-fluoropropyl)pyrrolidine was separated by chiral chromatography: Chiralpak AD 20 μm (250×4.6 mm) eluting with heptane/EtOH/TEA:95/05/0.1 to give 510 mg of Trans isomer (E)-3-(4-bromo-2-fluorobenzylidene)-1-(3-fluoropropyl)pyrrolidine and 248 mg of Cis isomer (Z)-3-(4-bromo-2-fluorobenzylidene)-1-(3-fluoropropyl)pyrrolidine.

LC/MS (m/z, MH+): 316

### Intermediate 18: [1-(3-Fluoropropyl)pyrrolidin-3-yl]-[4-(4,4,5,5-tetramethyl-1,3,2-dioxaborolan-2-yl)phenyl]methanone

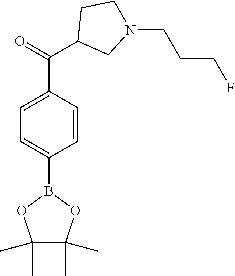 [SMILES: CC1(C)OB(c2ccc(C(=O)C3CCN(CCCF)C3)cc2)OC1(C)C]

Intermediate 18 was prepared following a similar procedure to that of Intermediate 14 from (4-bromophenyl)-[1-(3-fluoropropyl)pyrrolidin-3-yl]methanone (Step 2 of Intermediate 5) to give 2.4 g (100%) of [1-(3-fluoropropyl)pyrrolidin-3-yl]-[4-(4,4,5,5-tetramethyl-1,3,2-dioxaborolan-2-yl)phenyl]methanone.

LC/MS (m/z, MH+): 362

### Method A

### Example 1: (Z)-8-(2,4-Dichlorophenyl)-9-(4-((1-(3-fluoropropyl)pyrrolidin-3-ylidene)methyl)phenyl)-6,7-dihydro-5H-benzo[7]annulene-3-carboxylic acid, hydrochloride

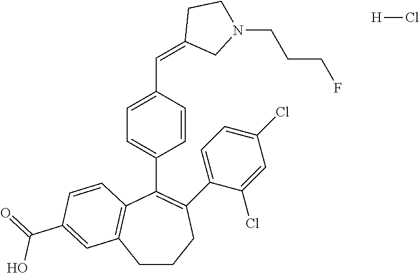 [SMILES: O=C(O)c1ccc2c(c1)CCCC(c1ccc(Cl)cc1Cl)=C2c1ccc(/C=C2/CCN(CCCF)C2)cc1 ; Cl]

### Step 1: Methyl (Z)-8-(2,4-dichlorophenyl)-9-(4-((1-(3-fluoropropyl)pyrrolidin-3-ylidene)methyl)phenyl)-6,7-dihydro-5H-benzo[7]annulene-3-carboxylate

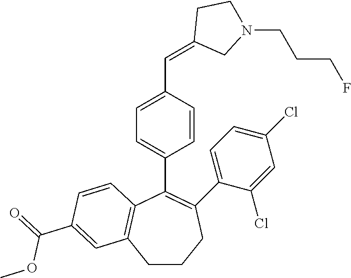 [SMILES: COC(=O)c1ccc2c(c1)CCCC(c1ccc(Cl)cc1Cl)=C2c1ccc(/C=C2/CCN(CCCF)C2)cc1]

A mixture of methyl 8-(2,4-dichlorophenyl)-9-(4,4,5,5-tetramethyl-1,3,2-dioxaborolan-2-yl)-6,7-dihydro-5H-benzo[7]annulene-3-carboxylate (Intermediate 2) (261.8 mg, 0.23 mmol), (3Z)-3-[(4-bromophenyl)methylene]-1-(3-fluoropropyl)pyrrolidine (Intermediate 6) (150 mg, 0.50 mmol), Cs₂CO₃ (426 mg, 1.31 mmol), and Pd(dppf)Cl₂, complex with DCM (25.8 mg, 0.03 mmol) in dioxane (4 ml) and water (1 ml) was heated to reflux 1 hour. After cooling to RT, water (10 ml) and EtOAc (20 ml) were added. After decantation, the organic phase was dried over Na₂SO₄, filtered, concentrated under reduced pressure, and the residue obtained was purified by flash chromatography eluting with cyclohexane/EtOAc 50/50 to give 131 mg (46%) of methyl (Z)-8-(2,4-dichlorophenyl)-9-(4-((1-(3-fluoropropyl)pyrrolidin-3-ylidene)methyl)phenyl)-6,7-dihydro-5H-benzo[7]annulene-3-carboxylate.

LC/MS (m/z, MH+): 564

### Step 2: (Z)-8-(2,4-Dichlorophenyl)-9-(4-((1-(3-fluoropropyl)pyrrolidin-3-ylidene)methyl)phenyl)-6,7-dihydro-5H-benzo[7]annulene-3-carboxylic acid, hydrochloride

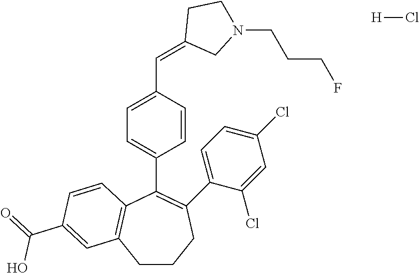 [SMILES: O=C(O)c1ccc2c(c1)CCCC(c1ccc(Cl)cc1Cl)=C2c1ccc(/C=C2/CCN(CCCF)C2)cc1 ; Cl]

To a solution of methyl (Z)-8-(2,4-dichlorophenyl)-9-(4-((1-(3-fluoropropyl)pyrrolidin-3-ylidene)methyl)phenyl)-6,7-dihydro-5H-benzo[7]annulene-3-carboxylate (131 mg, 0.22 mmol) in MeOH (2 ml), THF (1 ml) and water (0.5 ml) was added LiOH (53.86 mg, 2.25 mmol) and the reaction mixture was stirred at RT for 24 hours. The reaction mixture was concentrated under reduced pressure. To the residue obtained, addition of water (and HCl 1N till pH 1-2. The solid formed was filtered, washed with Et₂O and oven dried to give 81.5 mg (62%) of (Z)-8-(2,4-dichlorophenyl)-9-(4-((1-(3-fluoropropyl)pyrrolidin-3-ylidene)methyl)phenyl)-6,7-dihydro-5H-benzo[7]annulene-3-carboxylic acid, hydrochloride.

### Method B

### Example 2: (Z)-8-(2-Chloro-4-fluorophenyl)-9-(4-((1-(3,3-fluoropropyl)pyrrolidin-3-ylidene)methyl)phenyl)-6,7-dihydro-5H-benzo[7]annulene-3-carboxylic acid

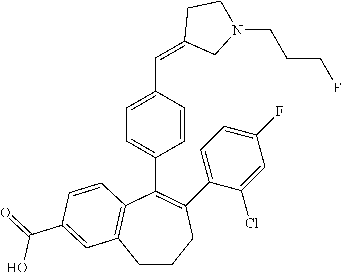 [SMILES: O=C(O)c1ccc2c(c1)CCCC(c1ccc(F)cc1Cl)=C2c1ccc(/C=C2/CCN(CCCF)C2)cc1]

### Step 1: Methyl (Z)-8-(2-chloro-4-fluorophenyl)-9-(4-((1-(3-fluoropropyl)pyrrolidin-3-ylidene)methyl)phenyl)-6,7-dihydro-5H-benzo[7]annulene-3-carboxylate

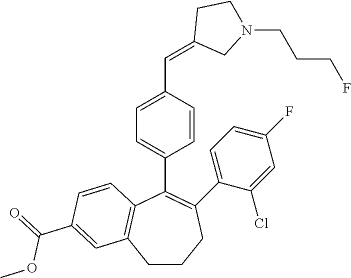 [SMILES: COC(=O)c1ccc2c(c1)CCCC(c1ccc(F)cc1Cl)=C2c1ccc(/C=C2/CCN(CCCF)C2)cc1]

A mixture of methyl (Z)-8-bromo-9-(4-((1-(3-fluoropropyl)pyrrolidin-3-ylidene)methyl)phenyl)-6,7-dihydro-5H-benzo[7]annulene-3-carboxylate (Intermediate 9) (87 mg, 0.17 mmol), (2-chloro-4-fluoro-phenyl)boronic acid (36.5 mg, 0.21 mmol), Cs₂CO₃ (148 mg, 0.45 mmol), and Pd(dppf)Cl₂, complex with DCM (8.55 mg, 0.01 mmol) in dioxane (4 ml) and water (1 ml) was heated at reflux for 1 hour. After cooling to RT, the reaction mixture was quenched with saturated solution of NH₄Cl(5 ml) and EtOAc (20 ml). After decantation, the organic phase was dried over Na₂SO₄, filtered, concentrated under reduced pressure and the residue was purified by flash chromatography eluting with a gradient of DCM/MeOH from 100/00 to 80/20 to give 53 mg (55%) of methyl (Z)-8-(2-chloro-4-fluorophenyl)-9-(4-((1-(3-fluoropropyl)pyrrolidin-3-ylidene)methyl)phenyl)-6,7-dihydro-5H-benzo[7]annulene-3-carboxylate.

LC/MS (m/z, MH+): 548

### Step 2: (Z)-8-(2-Chloro-4-fluorophenyl)-9-(4-((1-(3,3-fluoropropyl)pyrrolidin-3-ylidene)methyl)phenyl)-6,7-dihydro-5H-benzo[7]annulene-3-carboxylic acid

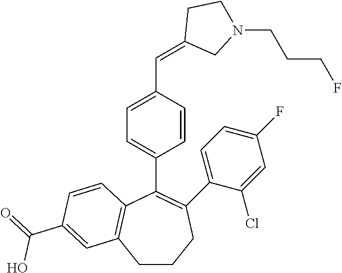 [SMILES: O=C(O)c1ccc2c(c1)CCCC(c1ccc(F)cc1Cl)=C2c1ccc(/C=C2/CCN(CCCF)C2)cc1]

Step 2 of Example 2 was prepared following a similar procedure to that of step 2 of Example 1 from methyl (Z)-8-(2-chloro-4-fluorophenyl)-9-(4-((1-(3-fluoropropyl)pyrrolidin-3-ylidene)methyl)phenyl)-6,7-dihydro-5H-benzo[7]annulene-3-carboxylate to give 33 mg (66%) of (Z)-8-(2-chloro-4-fluorophenyl)-9-(4-((1-(3,3-fluoropropyl)pyrrolidin-3-ylidene)methyl)phenyl)-6,7-dihydro-5H-benzo[7]annulene-3-carboxylic acid.

### Method C

### Example 24: (Z)-6-(2,4-Dichlorophenyl)-5-(4-((1-(3-fluoropropyl)pyrrolidin-3-ylidene)methyl)phenyl)-7,8-dihydronaphthalene-2-carboxylic acid, hydrochloride

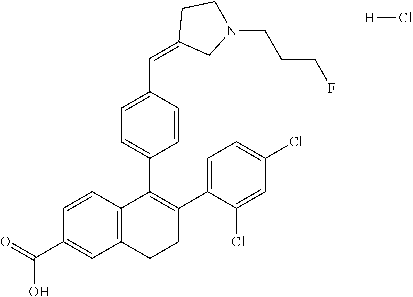 [SMILES: O=C(O)c1ccc2c(c1)CCC(c1ccc(Cl)cc1Cl)=C2c1ccc(/C=C2/CCN(CCCF)C2)cc1 ; Cl]

### Step 1: Methyl (Z)-6-(2,4-dichlorophenyl)-5-(4-((1-(3-fluoropropyl)pyrrolidin-3-ylidene)methyl)phenyl)-7,8-dihydronaphthalene-2-carboxylate

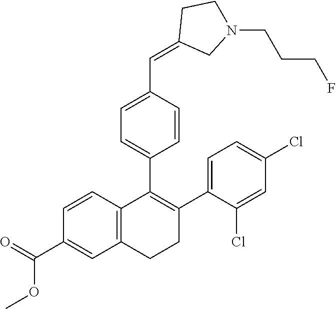 [SMILES: COC(=O)c1ccc2c(c1)CCC(c1ccc(Cl)cc1Cl)=C2c1ccc(/C=C2/CCN(CCCF)C2)cc1]

A mixture of methyl 6-(2,4-dichlorophenyl)-5-(trifluoromethylsulfonyloxy)-7,8-dihydronaphthalene-2-carboxylate (Intermediate 16) (280 mg, 0.28 mmol), (3Z)-1-(3-fluoropropyl)-3-[[4-(4,4,5,5-tetramethyl-1,3,2-dioxaborolan-2-yl)phenyl]methylene]pyrrolidine (Intermediate 14) (151 mg, 0.44 mmol), Cs₂CO₃ (486 mg, 1.49 mmol), and Pd(dppf)C₁₂, complex with DCM (43 mg, 0.05 mmol) in dioxane (3 ml) and water (1.2 1 ml) was heated at reflux for 30 minutes. After cooling to RT, water (2 ml) and DCM (10 ml) were added. After decantation on hydrophobic column, the organic phase was dried over Na₂SO₄, filtered, concentrated under reduced pressure, and the residue obtained was purified by flash chromatography eluting with a gradient of DCM/MeOH from 100/00 to 95/05 to give 123 mg (51%) of methyl (Z)-6-(2,4-dichlorophenyl)-5-(4-((1-(3-fluoropropyl)pyrrolidin-3-ylidene)methyl)phenyl)-7,8-dihydronaphthalene-2-carboxylate.

LC/MS (m/z, MH+): 550

### Step 2: (Z)-6-(2,4-Dichlorophenyl)-5-(4-((1-(3-fluoropropyl)pyrrolidin-3-ylidene)methyl)phenyl)-7,8-dihydronaphthalene-2-carboxylic acid, hydrochloride

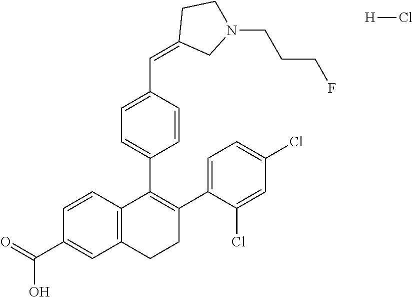 [SMILES: O=C(O)c1ccc2c(c1)CCC(c1ccc(Cl)cc1Cl)=C2c1ccc(/C=C2/CCN(CCCF)C2)cc1 ; Cl]

Step 2 of Example 24 was prepared following a similar procedure to that of step 2 of Example 1 from methyl (Z)-6-(2,4-dichlorophenyl)-5-(4-((1-(3-fluoropropyl)pyrrolidin-3-ylidene)methyl)phenyl)-7,8-dihydronaphthalene-2-carboxylate to give 113 mg (88%) of (Z)-6-(2,4-dichlorophenyl)-5-(4-((1-(3-fluoropropyl)pyrrolidin-3-ylidene)methyl)phenyl)-7,8-dihydronaphthalene-2-carboxylic acid, hydrochloride.

### Method D

### Example 16: (Z)-8-(2,4-Dichlorophenyl)-9-(4-((1-(3-fluoropropyl)pyrrolidin-3-ylidene)methyl)phenyl)-6,7-dihydro-5H-benzo[7]annulen-3-ol, hydrochloride

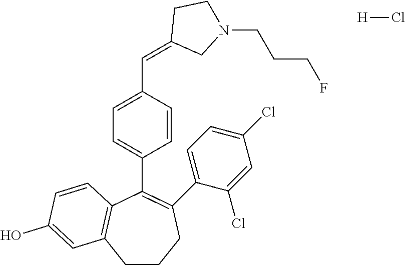 [SMILES: Oc1ccc2c(c1)CCCC(c1ccc(Cl)cc1Cl)=C2c1ccc(/C=C2/CCN(CCCF)C2)cc1 ; Cl]

### Step 1: (Z)-8-(2,4-Dichlorophenyl)-9-(4-((1-(3-fluoropropyl)pyrrolidin-3-ylidene)methyl)phenyl)-6,7-dihydro-5H-benzo[7]annulen-3-yl pivalate

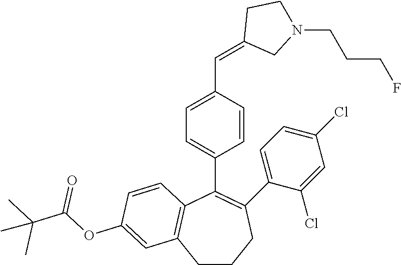 [SMILES: CC(C)(C)C(=O)Oc1ccc2c(c1)CCCC(c1ccc(Cl)cc1Cl)=C2c1ccc(/C=C2/CCN(CCCF)C2)cc1]

A mixture of 8-(2,4-dichlorophenyl)-9-(((trifluoromethyl)sulfonyl)oxy)-6,7-dihydro-5H-benzo[7]annulen-3-yl pivalate (Intermediate 13) (60 mg, 0.11 mmol), (3Z)-1-(3-fluoropropyl)-3-[[4-(4,4,5,5-tetramethyl-1,3,2-dioxaborolan-2-yl)phenyl]methylene]pyrrolidine (Intermediate 14) (40 mg, 0.11 mmol), Cs₂CO₃ (91 mg, 0.28 mmol), and Pd(dppf)Cl₂, complex with DCM (6 mg, 0.008 mmol) in dioxane (2 ml) and water (0.5 1 ml) was heated to reflux for 30 minutes. After cooling to RT, water (5 ml) and EtOAc (10 ml) were added. After decantation, the organic phase was dried over Na₂SO₄, filtered, concentrated under reduced pressure, and the residue obtained was purified by flash chromatography eluting with a gradient of cyclohexane/EtOAc from 90/10 to 80/20 to give 31 mg (46%) of (Z)-8-(2,4-dichlorophenyl)-9-(4-((1-(3-fluoropropyl)pyrrolidin-3-ylidene)methyl)phenyl)-6,7-dihydro-5H-benzo[7]annulen-3-yl pivalate.

LC/MS (m/z, MH+): 607

### Step 2: (Z)-8-(2,4-Dichlorophenyl)-9-(4-((1-(3-fluoropropyl)pyrrolidin-3-ylidene)methyl)phenyl)-6,7-dihydro-5H-benzo[7]annulen-3-ol, hydrochloride

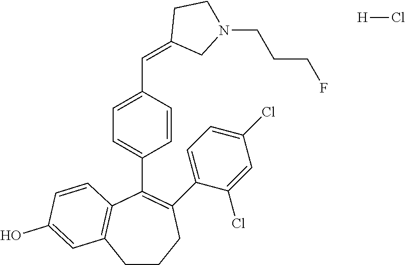 [SMILES: Oc1ccc2c(c1)CCCC(c1ccc(Cl)cc1Cl)=C2c1ccc(/C=C2/CCN(CCCF)C2)cc1 ; Cl]

To a solution of (Z)-8-(2,4-dichlorophenyl)-9-(4-((1-(3-fluoropropyl)pyrrolidin-3-ylidene)methyl)phenyl)-6,7-dihydro-5H-benzo[7]annulen-3-yl pivalate (31 mg, 0.04 mmol) in MeOH (2 ml) was added NaOH (6.8 mg, 0.17 mmol) and the reaction mixture was stirred at RT for 30 minutes. The reaction mixture was concentrated under reduced pressure. To the residue obtained, water (5 ml) and HCl 1N till pH 1-2 were added followed by DCM (10 ml). After decantation, the organic phase was dried over Na₂SO₄, filtered, concentrated under reduced pressure, and the residue obtained was purified by flash chromatography eluting with a gradient of DCM/MeOH from 100/00 to 95/05 to give 5.6 mg (17%) of (Z)-8-(2,4-dichlorophenyl)-9-(4-((1-(3-fluoropropyl)pyrrolidin-3-ylidene)methyl)phenyl)-6,7-dihydro-5H-benzo[7]annulen-3-ol, hydrochloride.

### Method E

### Example 30: (Z)-4-(2,4-Dichlorophenyl)-5-(4-((1-(3-fluoropropyl)pyrrolidin-3-ylidene)methyl)phenyl)-2,3-dihydrobenzo[b]thiepin-8-ol

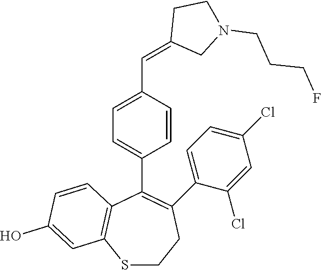 [SMILES: Oc1ccc2c(c1)SCCC(c1ccc(Cl)cc1Cl)=C2c1ccc(/C=C2/CCN(CCCF)C2)cc1]

### Step 1: 5-(4-(1-(3-Fluoropropyl)pyrrolidine-3-carbonyl)phenyl)-2,3-dihydrobenzo[b]thiepin-8-yl pivalate

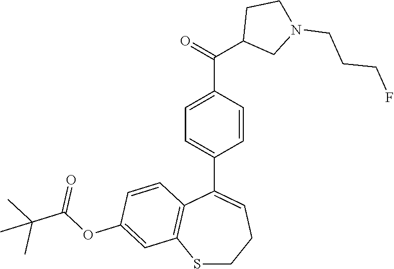 [SMILES: CC(C)(C)C(=O)Oc1ccc2c(c1)SCCC=C2c1ccc(C(=O)C2CCN(CCCF)C2)cc1]

Step 1 of Example 30 was prepared following a similar procedure to that of step 1 of Example 16 from 5-(((trifluoromethyl)sulfonyl)oxy)-2,3-dihydrobenzo[b]thiepin-8-yl pivalate (prepared according to WO2018091153) and [1-(3-fluoropropyl)pyrrolidin-3-yl]-[4-(4,4,5,5-tetramethyl-1,3,2-dioxaborolan-2-yl)phenyl]methanone (Intermediate 18) to give 2.7 g (86%) of 5-(4-(1-(3-fluoropropyl)pyrrolidine-3-carbonyl)phenyl)-2,3-dihydrobenzo[b]thiepin-8-yl pivalate.

LC/MS (m/z, MH+): 496

### Step 2: 5-(4-((1-(3-Fluoropropyl)pyrrolidin-3-yl)(hydroxy)methyl)phenyl)-2,3-dihydrobenzo[b]thiepin-8-yl pivalate

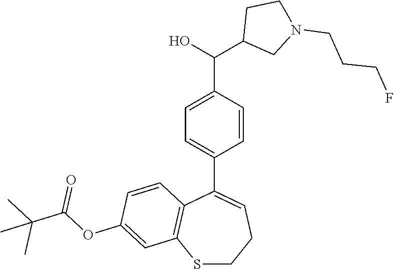 [SMILES: CC(C)(C)C(=O)Oc1ccc2c(c1)SCCC=C2c1ccc(C(O)C2CCN(CCCF)C2)cc1]

Step 2 of Example 30 was prepared following a similar procedure to that of Step 3 of Intermediate 5 from 5-(4-(1-(3-fluoropropyl)pyrrolidine-3-carbonyl)phenyl)-2,3-dihydrobenzo[b]thiepin-8-yl pivalate to give 980 mg (36%) of 5-(4-((1-(3-fluoropropyl)pyrrolidin-3-yl)(hydroxy)methyl)phenyl)-2,3-dihydrobenzo[b]thiepin-8-yl pivalate.

LC/MS (m/z, MH+): 498

### Step 3: 4-Bromo-5-(4-((1-(3-fluoropropyl)pyrrolidin-3-yl)(hydroxy)methyl)phenyl)-2,3-dihydrobenzo[b]thiepin-8-yl pivalate

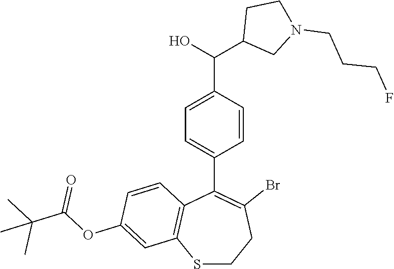 [SMILES: CC(C)(C)C(=O)Oc1ccc2c(c1)SCCC(Br)=C2c1ccc(C(O)C2CCN(CCCF)C2)cc1]

Step 3 of Example 30 was prepared following a similar procedure to that of Step 2 of Intermediate 9 from 5-(4-((1-(3-fluoropropyl)pyrrolidin-3-yl)(hydroxy)methyl)phenyl)-2,3-dihydrobenzo[b]thiepin-8-yl pivalate to give 650 mg (56%) of 4-bromo-5-(4-((1-(3-fluoropropyl)pyrrolidin-3-yl)(hydroxy)methyl)phenyl)-2,3-dihydrobenzo[b]thiepin-8-yl pivalate.

LC/MS (m/z, MH+): 576

### Step 4: 4-(2,4-Dichlorophenyl)-5-(4-((1-(3-fluoropropyl)pyrrolidin-3-yl)(hydroxy)methyl)phenyl)-2,3-dihydrobenzo[b]thiepin-8-yl pivalate

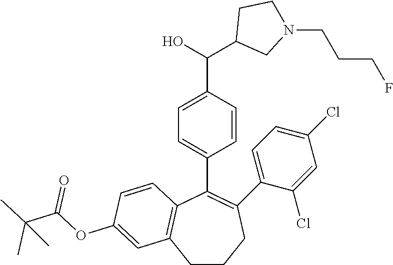 [SMILES: CC(C)(C)C(=O)Oc1ccc2c(c1)CCCC(c1ccc(Cl)cc1Cl)=C2c1ccc(C(O)C2CCN(CCCF)C2)cc1]

Step 4 of Example 30 was prepared following a similar procedure to that of Step 1 of Example 2 from 4-bromo-5-(4-((1-(3-fluoropropyl)pyrrolidin-3-yl)(hydroxy)methyl)phenyl)-2,3-dihydrobenzo[b]thiepin-8-yl pivalate to give 700 mg (97%) of 4-(2,4-dichlorophenyl)-5-(4-((1-(3-fluoropropyl)pyrrolidin-3-yl)(hydroxy)methyl)phenyl)-2,3-dihydrobenzo[b]thiepin-8-yl pivalate.

LC/MS (m/z, MH+): 642

### Step 5: 4-(2,4-Dichlorophenyl)-5-(4-((1-(3-fluoropropyl)pyrrolidin-3-yl)(hydroxy)methyl)phenyl)-2,3-dihydrobenzo[b]thiepin-8-ol

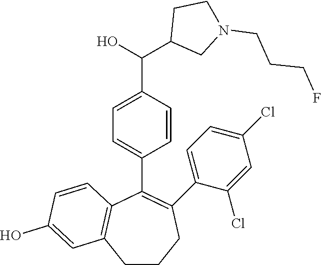 [SMILES: Oc1ccc2c(c1)CCCC(c1ccc(Cl)cc1Cl)=C2c1ccc(C(O)C2CCN(CCCF)C2)cc1]

To a solution of 4-(2,4-dichlorophenyl)-5-(4-((1-(3-fluoropropyl)pyrrolidin-3-yl)(hydroxy)methyl)phenyl)-2,3-dihydrobenzo[b]thiepin-8-yl pivalate (724 mg, 1.09 mmol) in MeOH (5 ml) was added NaOH 5N (0.65 ml, 3.3 mmol) and the reaction mixture was stirred at RT for 30 minutes. The reaction mixture was concentrated under reduced pressure. To the residue obtained, water (5 ml) and HCl 1N till pH 7 were added followed by DCM (20 ml). After decantation, the organic phase was dried over Na₂SO₄, filtered, concentrated under reduced pressure, and the residue obtained was purified by flash chromatography eluting with a gradient of DCM/MeOH from 100/00 to 90/10 to give 570 mg (91%) of 4-(2,4-dichlorophenyl)-5-(4-((1-(3-fluoropropyl)pyrrolidin-3-yl)(hydroxy)methyl)phenyl)-2,3-dihydrobenzo[b]thiepin-8-ol.

LC/MS (m/z, MH+): 558

### Step 5: (Z)-4-(2,4-Dichlorophenyl)-5-(4-((1-(3-fluoropropyl)pyrrolidin-3-ylidene)methyl)phenyl)-2,3-dihydrobenzo[b]thiepin-8-ol

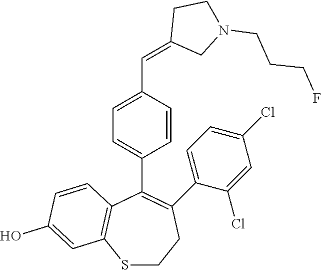 [SMILES: Oc1ccc2c(c1)SCCC(c1ccc(Cl)cc1Cl)=C2c1ccc(/C=C2/CCN(CCCF)C2)cc1]

To a mixture of 4-(2,4-dichlorophenyl)-5-(4-((1-(3-fluoropropyl)pyrrolidin-3-yl)(hydroxy)methyl)phenyl)-2,3-dihydrobenzo[b]thiepin-8-ol (560 mg, 1 mmol) in water (1.3 ml) cooled at 0° C. was added dropwise sulfuric acid (11 ml, 207 mmol). The cooling bath was removed, and the reaction mixture was stirred for 18 hour at RT and 1.5 hour at 50° C. The reaction mixture was poured into a mixture of ice and water (500 ml). Powder NaHCO₃ was added to pH 9 followed by EtOAc (300 ml) and the organic phase was separated and dried over Na₂SO₄, filtered, concentrated under reduced pressure and the residue purified by flash chromatography eluting with a gradient of DCM/MeOH from 100/00 to 95/05 to give 11 mg (2%) of 4-(2,4-dichlorophenyl)-5-(4-((1-(3-fluoropropyl)pyrrolidin-3-ylidene)methyl)phenyl)-2,3-dihydrobenzo[b]thiepin-8-ol as a mixture of (Z) and (E) isomers.

The mixture of (Z) and (E) isomers of 4-(2,4-dichlorophenyl)-5-(4-((1-(3-fluoropropyl)pyrrolidin-3-ylidene)methyl)phenyl)-2,3-dihydrobenzo[b]thiepin-8-ol was separated on column I-Cellulose 5 μm (eq phenomenex Chiralpak IC) (0.3×25 cm), eluting with heptane/EtOH/TEA (90/10/0.1) to give 2.7 mg of (Z) isomer (Z)-4-(2,4-dichlorophenyl)-5-(4-((1-(3-fluoropropyl)pyrrolidin-3-ylidene)methyl)phenyl)-2,3-dihydrobenzo[b]thiepin-8-ol and 4.1 mg of (E) isomer (E)-4-(2,4-dichlorophenyl)-5-(4-((1-(3-fluoropropyl)pyrrolidin-3-ylidene)methyl)phenyl)-2,3-dihydrobenzo[b]thiepin-8-ol.

The compounds according to Table 1a above were subjected to pharmacological tests for determining their degradation effects on estrogen receptors.

### Test: Estrogen Receptor Degradation Activity

Said test involves measuring the in vitro degradation activity of the compounds of the Table 1a.

The measurements of the degradation activities were made using a breast cancer cell ERα in cell western assay as described hereunder.

MCF7 cells (ATCC) were seeded in 384 wells microplate (collagen coated) at a concentration of 10000 cells/30 μL per well in red phenol free MEM alpha medium (invitrogen) containing 5% charcoal dextran striped FBS. The following day, 9 points serial 1:5 dilution of each compound was added to the cells in 2.5 μL at final concentrations ranging from 0.3-0.0000018 μM (in table 2), or 0.1 μM for fulvestrant (using as positive control). At 4 hours post compound addition the cells were fixed by adding 25 μL of formalin (final concentration 5% formalin containing 0.1% triton) for 10 minutes at room temperature and then washed twice with PBS. Then, 50 μL of LI-COR blocking buffer containing 0.1% Triton was added to plate for 30 minutes at room temperature. LI-COR blocking buffer was removed and cells were incubated overnight at cold room with 50 μL anti-ER rabbit monoclonal antibody (Thermo scientific MA1-39540) diluted at 1:1000 in LI-COR blocking buffer containing 0.1% tween-20. Wells which were treated with blocking buffer but no antibody were used as background control. Wells were washed twice with PBS (0.1% tween-20) and incubated at 37° C. for 60 minutes in LI-COR (0.1% tween-20) containing goat anti-rabbit antibody Alexa 488 (1:1000) and Syto-64 a DNA dye (2 μM final concentration). Cells were then washed 3 times in PBS and scanned in ACUMEN explorer (TTP-Labtech). Integrated intensities in the green fluorescence and red fluorescence were measured to determine the levels of ERα and DNA respectively.

The degradation activity with respect to estrogen receptors in this test is given by the concentration which degrades 50% of the estrogen receptor (or IC₅₀) in nM.

The % of ERα levels decrease were determined as follows: % inhibition=100*(1−(sample−fulvestrant:DMSO−fulvestrant)).

The Table 2 below indicates the estrogen receptor degradation activity results for the compounds of Table 1a tested at 0.3 μM, and demonstrates that said compounds have a significant degradation activity on estrogen receptors.

TABLE 2

| | | % |
|---|---|---|
| | Degradation | Degradation |
| Compound No. | IC₅₀ (nM) | At 0.3 μM |
| 1 | 0.5 | 96 |
| 2 | 0.4 | 91 |
| 3 | 0.6 | 95 |
| 4 | 2.4 | 73 |
| 5 | 0.8 | 88 |
| 6 | 0.8 | 96 |
| 7 | 5.7 | 82 |
| 8 | 0.5 | 90 |
| 9 | 0.7 | 93 |
| 10 | 0.7 | 92 |
| 11 | 0.5 | 93 |
| 12 | 1 | 85 |
| 13 | 1.6 | 86 |
| 14 | 0.7 | 92 |
| 15 | 0.9 | 88 |
| 16 | 1.5 | 76 |
| 17 | 3.1 | 61 |
| 18 | 2.4 | 86 |
| 19 | 2.8 | 87 |
| 20 | 1.8 | 85 |
| 21 | 1 | 94 |
| 22 | 0.4 | 89 |
| 23 | 0.6 | 96 |
| 24 | 3.2 | 91 |
| 25 | 1 | 91 |
| 26 | 1.5 | 92 |
| 27 | 1 | 93 |
| 28 | 1.2 | 93 |
| 29 | 1.3 | 92 |
| 30 | 1.8 | 90 |

It is therefore apparent that the tested compounds have degradation activities for estrogen receptors, with IC50 less than 1 μM and with degradation levels greater than 50%. The compounds of formula (I) can therefore be used for preparing medicaments, especially medicaments which are degraders of estrogen receptors.

Accordingly, also provided herein are medicaments which comprise a compound of the formula (I), or a pharmaceutically acceptable salt thereof.

Herein are also provided the compounds of formula (I) defined above, or pharmaceutically acceptable salts thereof, for use as medicines.

Herein are also provided the compounds of formula (I) defined above, or pharmaceutically acceptable salt thereof, for use in therapy, especially as inhibitors and degraders of estrogen receptors.

Herein are also provided the compounds of formula (I) defined above, or a pharmaceutically acceptable salts thereof, for use in the treatment of ovulatory dysfunction, cancer, endometriosis, osteoporosis, benign prostatic hypertrophy or inflammation.

A particular aspect is a compound of formula (I) defined above, or a pharmaceutically acceptable salt thereof, for use in the treatment of cancer.

In an embodiment, the cancer is a hormone dependent cancer.

In another embodiment, the cancer is an estrogen receptor dependent cancer, particularly the cancer is an estrogen receptor a dependent cancer.

In another embodiment, the cancer is selected from breast, ovarian, endometrial, prostate, uterine, cervical and lung cancer, or a metastasis thereof.

In another embodiment, the metastasis is a cerebral metastasis.

In another embodiment, the cancer is breast cancer. Particularly, the breast cancer is an estrogen receptor positive breast cancer (ERα positive breast cancer).

In another embodiment, the cancer is resistant to anti-hormonal treatment.

In a further embodiment, the compound of formula (I) is as used as single agent or in combination with other agents such as CDK4/6, mTOR or PI3K inhibitors.

According to another aspect, herein is provided a method of treating the pathological conditions indicated above, comprising administering to a subject in need thereof a therapeutically effective amount of a compound of formula (I), or a pharmaceutically acceptable salt thereof. In an embodiment of this method of treatment, the subject is a human.

Herein is also provided the use of a compound of the formula (I), or a pharmaceutically acceptable salt thereof, for the manufacture of a medicament useful in treating any of the pathological conditions indicated above, more particularly useful in treating cancer.

Herein are also provided the pharmaceutical compositions comprising as active principle a compound of formula (I). These pharmaceutical compositions comprise an effective dose of at least one compound of formula (I), or a pharmaceutically acceptable salt thereof, and also at least one pharmaceutically acceptable excipient.

The said excipients are selected, in accordance with the pharmaceutical form and method of administration desired, from the customary excipients, which are known to a person skilled in the art.

In the pharmaceutical compositions for oral, sublingual, subcutaneous, intramuscular, intravenous, topical, local, intra-tracheal, intranasal, transdermal or rectal administration, the active principle of formula (I) above, or its base, acid, zwitterion or salt thereof, may be administered in a unit administration form, in a mixture with conventional pharmaceutical excipients, to animals and to human beings for the treatment of the above disorders or diseases.

The unit administration forms appropriate include oral forms such as tablets, soft or hard gel capsules, powders, granules and oral solutions or suspensions, sublingual, buccal, intra-tracheal, intra-ocular and intra-nasal administration forms, forms for inhalative, topical, transdermal, subcutaneous, intra-muscular or intravenous administration, rectal administration forms and implants. For topical application it is possible to use the compounds of formula (I) in creams, gels, ointments or lotions.

As an example, a unit administration form of a compound of formula (I) in tablet form may comprise the following components:

| | | |
|---|---|---|
| Compound of formula (I) | 50.0 | mg |
| Mannitol | 223.75 | mg |
| Sodium croscarmellose | 6.0 | mg |
| Corn starch | 15.0 | mg |
| Hydroxypropylmethylcellulose | 2.25 | mg |
| Magnesium stearate | 3.0 | mg |

There may be particular cases in which higher or lower dosages are appropriate. According to usual practice, the dosage that is appropriate for each patient is determined by the doctor according to the mode of administration and the weight and response of the said patient.

1. A compound of the formula (I) or a pharmaceutically acceptable salt thereof: 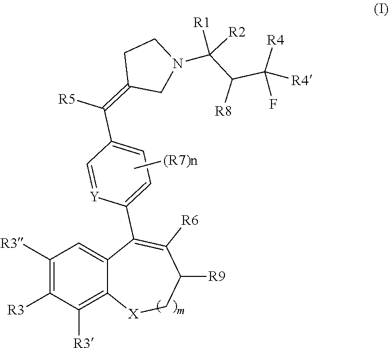
wherein:
R1 and R2 independently represent a hydrogen atom or a deuterium atom;
R3 represents a hydrogen atom, a —COOH group or a —OH group;
R3′ and R3″ independently represent a hydrogen atom, a methyl group, a methoxy group, a chlorine atom, a fluorine atom or a cyano group;
R4 and R4′ independently represent a hydrogen atom or a fluorine atom;
R5 represents a hydrogen atom, a fluorine atom or a (C₁-C₃)alkyl group;
R6 represents a group selected from:
a phenyl group, said phenyl group being optionally substituted by 1 to 3 substituents independently selected from a halogen atom; a (C₁-C₆)alkyl group optionally substituted with a cyano group or a —OH group; a (C₁-C₆)fluoroalkyl group; a (C₃-C₆)cycloalkyl group; a (C₁-C₆)alkoxy group; a (C₁-C₆)fluoroalkoxy group; a cyano group; a trifluoromethylsulfonyl group; a (C₁-C₄)alkylthio group; a (C₁-C₄)fluoroalkylthio group; a (C₁-C₄)alkylsulfonyl group and a —OH group;
a fused phenyl group, selected from phenyl groups fused with a (C₃-C₆)cycloalkyl, said (C₃-C₆)cycloalkyl optionally comprises an unsaturation, and wherein the fused phenyl group is optionally substituted with 1 to 3 substituents independently selected from a (C₁-C₃)alkyl group, a hydroxy group, a halogen atom, a (C₁-C₆)fluoroalkyl group and a (C₁-C₃)alkoxy group;
a bicyclic group comprising 5 to 12 carbon atoms, optionally comprising 1 to 2 unsaturations; optionally substituted with 1 to 4 substituents independently selected from: a fluorine atom, a —OH group, a (C₁-C₃)alkyl group, a (C₁-C₃)fluoroalkyl group, a (C₁-C₃)alkoxy group, a (C₁-C₃)fluoroalkoxy group and an oxo group;
a heteroaryl group comprising 2 to 9 carbon atoms and comprising from 1 to 3 heteroatoms independently selected from oxygen, nitrogen and sulfur, and at least 5 atoms including carbon atoms and heteroatoms, said heteroaryl group being optionally substituted with 1 to 3 substituents independently selected from a halogen atom, a (C₁-C₆)alkyl group, a (C₁-C₆)fluoroalkyl group, a (C₁-C₆)alkoxy group, a (C₁-C₆)fluoroalkoxy group, a cyano group, a carbamoyl group and a —OH group;
a cycloalkyl group comprising 3 to 7 carbon atoms, said cycloalkyl group being saturated or partially saturated and being optionally substituted with 1 to 4 substituents independently selected from:
a fluorine atom, a —OH group, a (C₁-C₃)alkyl group, a (C₁-C₃)fluoroalkyl group, a (C₁-C₃)alkoxy group, a (C₁-C₃)fluoroalkoxy group, an oxo group,
a (C₃-C₆)cycloalkyl group and a phenyl group, said (C₃-C₆)cycloalkyl or phenyl groups being optionally substituted with one or two halogen atom(s) or (C₁-C₃)alkyl group(s);
a (C₃-C₆)cycloalkyl(C₁-C₃)alkyl group, optionally substituted on the cycloalkyl with 1 to 4 substituents independently selected from: a fluorine atom, a —OH group, a (C₁-C₄)alkyl group, a (C₁-C₃)fluoroalkyl group, a (C₁-C₃)fluoroalkoxy group and an oxo group;
a 4 to 7 membered-heterocycloalkyl group comprising 1 or 2 heteroatoms independently selected from oxygen, nitrogen and sulfur, said heterocycloalkyl group being saturated or partially saturated and being optionally substituted with 1 to 3 substituents independently selected from: a fluorine atom, a (C₁-C₃)alkyl group, a (C₁-C₃)fluoroalkyl group, a (C₁-C₃)fluoroalkoxy group, an oxo group, a (C₁-C₃)alkoxy group, and a —OH group;
a (C₁-C₆)alkyl group, said alkyl group being optionally substituted with 1 to 4 substituents independently selected from: a fluorine atom, a (C₁-C₃)alkoxy group, a (C₁-C₃)fluoroalkoxy group and a —OH group; and
a phenyl(C₁-C₂)alkyl group, said phenyl group being optionally substituted with 1 to 3 substituents independently selected from a halogen atom; a (C₁-C₃)alkyl group; a (C₁-C₃)fluoroalkyl group; a (C₁-C₃)alkoxy group; a (C₁-C₃)fluoroalkoxy group; a cyano group; and a —OH group;
X represents —CH₂—, —O— or —S—;
Y represents —CH═, —N═ or —CR″═, wherein R″ represents a (C₁-C₃)alkyl group, a halogen atom, a cyano group, or a (C₁-C₃)fluoroalkyl group;
R7 independently represents a (C₁-C₃)alkyl group, a halogen atom, a cyano group, or a (C₁-C₃)fluoroalkyl group;
R8 represents a hydrogen atom or a fluorine atom;
R9 represents a hydrogen atom, a (C₁-C₃)alkyl group or a cyclopropyl;
n is 0, 1 or 2; and
m is 0 or 1.

2. The compound of formula (I) according to claim 1, or a pharmaceutically acceptable salt thereof, characterized in that R1 and R2 are a hydrogen atom.

3. The compound of formula (I) according to claim 1, or a pharmaceutically acceptable salt thereof, characterized in that R3 is a —COOH group.

4. The compound of formula (I) according to claim 1, or a pharmaceutically acceptable salt thereof, characterized in that R3′ and R3″ both represent a hydrogen atom.

5. The compound of formula (I) according to claim 1, or a pharmaceutically acceptable salt thereof, characterized in that R4 and R4′ both represent a hydrogen atom; or one of R4 and R4′ represents a hydrogen atom and the other a fluorine atom.

6. The compound of formula (I) according to claim 1, or a pharmaceutically acceptable salt thereof, characterized in that X represents —CH₂—.

7. The compound of formula (I) according to claim 1, or a pharmaceutically acceptable salt thereof, characterized in that R5 represents a hydrogen atom.

8. The compound of formula (I) according to claim 1, or a pharmaceutically acceptable salt thereof, characterized in that R6 represents a phenyl group, said phenyl group being optionally substituted with 1 to 3 substituents independently selected from a halogen atom; a (C₁-C₆)alkyl group and a (C₁-C₆)fluoroalkyl group.

9. The compound of formula (I) according to claim 1, or a pharmaceutically acceptable salt thereof, characterized in that n is 0 or 1 and R7 is a fluorine atom.

10. The compound of formula (I) according to claim 1, or a pharmaceutically acceptable salt thereof, wherein Y represents —CH═.

11. The compound of formula (I) according to claim 1, or a pharmaceutically acceptable salt thereof, wherein m is 1.

12. The compound of formula (I) according to claim 1, or a pharmaceutically acceptable salt thereof, characterized in that said compound is selected from the following compounds:
(Z)-8-(2,4-dichlorophenyl)-9-(4-((1-(3,3-fluoropropyl)pyrrolidin-3-ylidene)methyl)phenyl)-6,7-dihydro-5H-benzo[7]annulene-3-carboxylic acid hydrochloride (1),
(Z)-8-(2-chloro-4-fluorophenyl)-9-(4-((1-(3,3-fluoropropyl)pyrrolidin-3-ylidene)methyl)phenyl)-6,7-dihydro-5H-benzo[7]annulene-3-carboxylic acid (2),
(Z)-4-(2,4-dichlorophenyl)-5-(4-((1-(3-fluoropropyl)pyrrolidin-3-ylidene)methyl)phenyl)-2,3-dihydrobenzo[b]oxepine-8-carboxylic acid hydrochloride (3),
Z)-8-(2,4-dichlorophenyl)-9-(4-((1-(3,3-difluoropropyl)pyrrolidin-3-ylidene)methyl)phenyl)-6,7-dihydro-5H-benzo[7]annulene-3-carboxylic acid (4),
(Z)-8-(2,4-difluorophenyl)-9-(4-((1-(3,3-difluoropropyl)pyrrolidin-3-ylidene)methyl)phenyl)-6,7-dihydro-5H-benzo[7]annulene-3-carboxylic acid hydrochloride (5),
(Z)-8-(4-fluoro-2-methylphenyl)-9-(4-((1-(3-fluoropropyl)pyrrolidin-3-ylidene)methyl)phenyl)-6,7-dihydro-5H-benzo[7]annulene-3-carboxylic acid (6),
(Z)-3-(4-(8-(2-chlorophenyl)-6,7-dihydro-5H-benzo[7]annulen-9-yl)benzylidene)-1-(3-fluoropropyl)pyrrolidine (7),
(Z)-8-(4-fluoro-2-(trifluoromethyl)phenyl)-9-(4-((1-(3-fluoropropyl)pyrrolidin-3-ylidene)methyl)phenyl)-6,7-dihydro-5H-benzo[7]annulene-3-carboxylic acid (8),
(Z)-8-(2,4-difluorophenyl)-9-(4-((1-(3,3-fluoropropyl)pyrrolidin-3-ylidene)methyl)phenyl)-6,7-dihydro-5H-benzo[7]annulene-3-carboxylic (9),
(Z)-8-(3-chloro-2-methylphenyl)-9-(4-((1-(3-fluoropropyl)pyrrolidin-3-ylidene)methyl)phenyl)-6,7-dihydro-5H-benzo[7]annulene-3-carboxylic acid (10),
(Z)-9-(4-((1-(3-fluoropropyl)pyrrolidin-3-ylidene)methyl)phenyl)-8-(3-methyl-2-(trifluoromethyl)phenyl)-6,7-dihydro-5H-benzo[7]annulene-3-carboxylic acid (11),
(Z)-9-(4-((1-(3,3-difluoropropyl)pyrrolidin-3-ylidene)methyl)phenyl)-8-(3-methyl-2-(trifluoromethyl)phenyl)-6,7-dihydro-5H-benzo[7]annulene-3-carboxylic acid (12),
(Z)-8-(3-chloro-2-methylphenyl)-9-(4-((1-(3,3-difluoropropyl)pyrrolidin-3-ylidene)methyl)phenyl)-6,7-dihydro-5H-benzo[7]annulene-3-carboxylic acid (13),
(Z)-9-(4-((1-(3-fluoropropyl)pyrrolidin-3-ylidene)methyl)phenyl)-8-(2-methyl-3-(trifluoromethyl)phenyl)-6,7-dihydro-5H-benzo[7]annulene-3-carboxylic acid (14),
(Z)-9-(4-((1-(3,3-difluoropropyl)pyrrolidin-3-ylidene)methyl)phenyl)-8-(4-fluoro-2-methylphenyl)-6,7-dihydro-5H-benzo[7]annulene-3-carboxylic acid (15),
(Z)-8-(2,4-dichlorophenyl)-9-(4-((1-(3-fluoropropyl)pyrrolidin-3-ylidene)methyl)phenyl)-6,7-dihydro-5H-benzo[7]annulen-3-ol hydrochloride (16),
(Z)-8-(2,4-dichlorophenyl)-9-(4-((1-(3-difluoropropyl)pyrrolidin-3-ylidene)methyl)phenyl)-6,7-dihydro-5H-benzo[7]annulen-3-ol hydrochloride (17),
(Z)-9-(4-((1-(3,3-difluoropropyl)pyrrolidin-3-ylidene)methyl)phenyl)-8-(4-fluoro-2-(trifluoromethyl)phenyl)-6,7-dihydro-5H-benzo[7]annulene-3-carboxylic acid (18),
(Z)-9-(4-((1-(3,3-difluoropropyl)pyrrolidin-3-ylidene)methyl)phenyl)-8-(2-methyl-3-(trifluoromethyl)phenyl)-6,7-dihydro-5H-benzo[7]annulene-3-carboxylic acid (19),
(Z)-9-(4-((1-(3,3-difluoropropyl)pyrrolidin-3-ylidene)methyl)phenyl)-8-(4-fluoro-2,6-dimethyl)phenyl)-6,7-dihydro-5H-benzo[7]annulene-3-carboxylic acid (20),
(Z)-8-(4-fluoro-2,6-dimethylphenyl)-9-(4-((1-(3-fluoropropyl)pyrrolidin-3-ylidene)methyl)phenyl)-6,7-dihydro-5H-benzo[7]annulene-3-carboxylic acid (21),
(Z)-9-(4-((1-(3-fluoropropyl)pyrrolidin-3-ylidene)methyl)phenyl)-8-(2-(trifluoromethyl)phenyl)-6,7-dihydro-5H-benzo[7]annulene-3-carboxylic acid (22),
(Z)-8-(2,4-dichlorophenyl)-9-(3-fluoro-4-((1-(3-fluoropropyl)pyrrolidin-3-ylidene)methyl)phenyl)-6,7-dihydro-5H-benzo[7]annulene-3-carboxylic acid (23),
(Z)-6-(2,4-dichlorophenyl)-5-(4-((1-(3-fluoropropyl)pyrrolidin-3-ylidene)methyl)phenyl)-7,8-dihydronaphthalene-2-carboxylic acid hydrochloride (24),
(Z)-9-(4-((1-(3-fluoropropyl)pyrrolidin-3-ylidene)methyl)phenyl)-8-(3-(trifluoromethyl)phenyl)-6,7-dihydro-5H-benzo[7]annulene-3-carboxylic acid (25),
(Z)-9-(4-((1-(3-fluoropropyl)pyrrolidin-3-ylidene)methyl)phenyl)-8-(4-(trifluoromethyl)phenyl)-6,7-dihydro-5H-benzo[7]annulene-3-carboxylic acid (26),
(Z)-8-(3-chlorophenyl)-9-(4-((1-(3-fluoropropyl)pyrrolidin-3-ylidene)methyl)phenyl)-6,7-dihydro-5H-benzo[7]annulene-3-carboxylic acid (27),
(Z)-8-(4-chlorophenyl)-9-(4-((1-(3-fluoropropyl)pyrrolidin-3-ylidene)methyl)phenyl)-6,7-dihydro-5H-benzo[7]annulene-3-carboxylic acid (28),
(Z)-8-(4-fluoro-2,5-dimethylphenyl)-9-(4-((1-(3-fluoropropyl)pyrrolidin-3-ylidene)methyl)phenyl)-6,7-dihydro-5H-benzo[7]annulene-3-carboxylic acid (29),
(Z)-4-(2,4-dichlorophenyl)-5-(4-((1-(3-fluoropropyl)pyrrolidin-3-ylidene)methyl)phenyl)-2,3-dihydrobenzo[b]thiepin-8-ol (30).

13. A process for preparing a compound of formula (I) as defined in claim 1, or a pharmaceutically acceptable salt thereof, wherein a compound of formula 1K 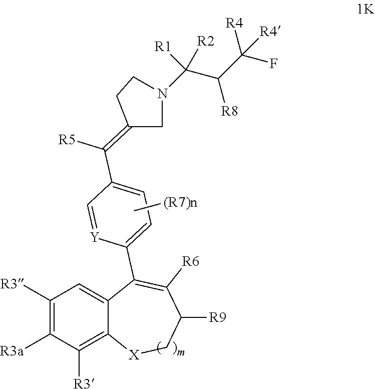
wherein R1, R2, R3′, R3″, R4, R4′, R5, R6, R7, R8, R9, m, n, X and Y are as defined in claim 1 and R3a is a hydrogen atom, a carboxylic ester, or protected OH, is converted to compound of formula (I), in presence of a source of hydroxide ions, said step being optionally preceded by a step for obtaining compound 1K, wherein a compound of formula 1P 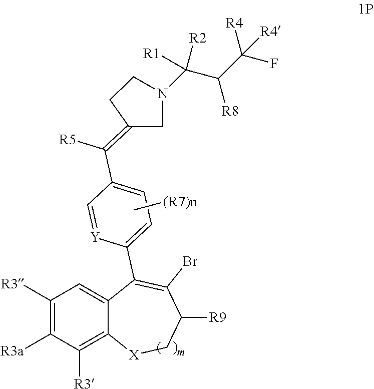
wherein, R1, R2, R3′, R3″, R4, R4′, R5, R7, R8, R9, m, n, X and Y are as defined in claim 1 and R3a is as defined above,
is subjected to a Suzuki coupling with a boronic reagent R6B(OR′)₂ or R6BF₃K, wherein —B(OR′)₂ is a boronic acid or a pinacolate ester and R6 is as defined in claim 1.

14. A process for preparing a compound of formula (I) as defined in claim 1, or a pharmaceutically acceptable salt thereof, wherein a compound of formula 1Pa 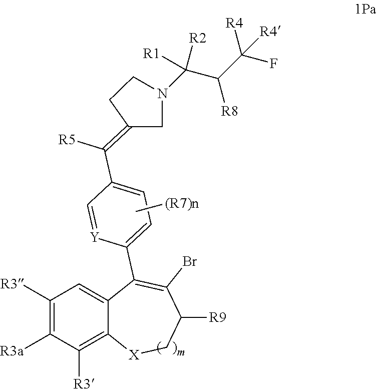
wherein R1, R2, R3 R3′, R3″, R4, R4′, R5, R7, R8, R9, m, n, X and Y are as defined in claim 1, is submitted to a Suzuki coupling with a boronic reagent R6B(OR′)₂ or R6BF₃K, wherein —B(OR′)₂ is a boronic acid or a pinacolate ester and R6 is defined as in claim 1, said step being optionally preceded by a step for obtaining compound 1Pa, wherein a compound of formula 1P 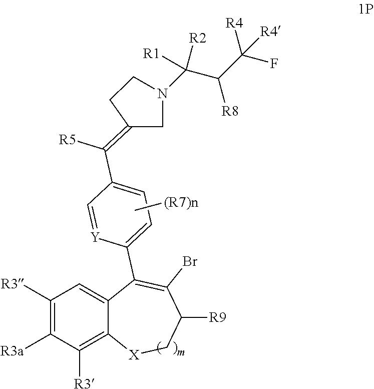
wherein R1, R2, R3′, R3″, R4, R4′, R5, R7, R8, R9, m, n, X and Y are as defined in claim 1 and R3a is a hydrogen atom, a carboxylic ester, or protected OH,
is converted to a compound 1 Pa in the presence of a source of hydroxide ions.

15. A compound selected from compounds of formula 1P, 1K and 1 Pa, or a pharmaceutically acceptable salt thereof: 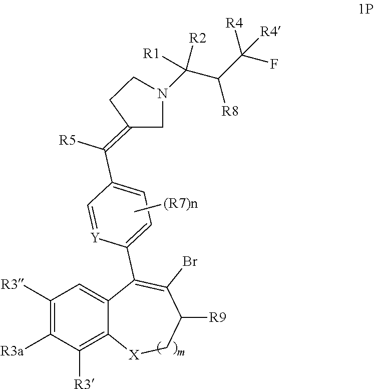 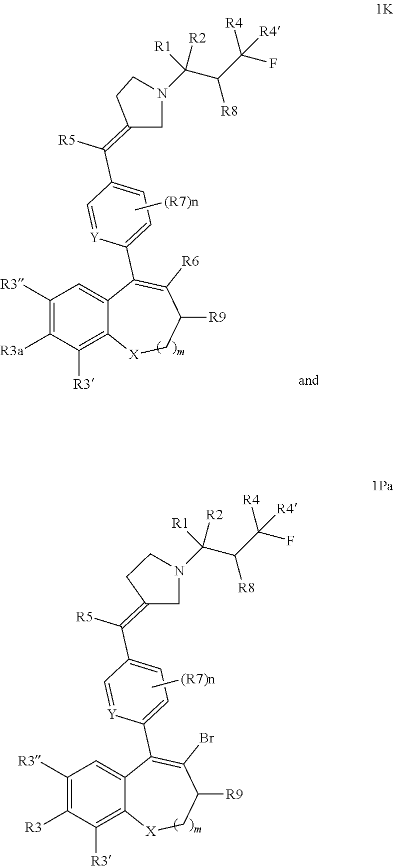
wherein:
R1 and R2 independently represent a hydrogen atom or a deuterium atom;
R3 represents a hydrogen atom, a —COOH group or a —OH group;
R3a is a hydrogen atom, a carboxylic ester, or protected OH:
R3′ and R3″ independently represent a hydrogen atom, a methyl group, a methoxy group, a chlorine atom, a fluorine atom or a cyano group;
R4 and R4′ independently represent a hydrogen atom or a fluorine atom;
R5 represents a hydrogen atom, a fluorine atom or a (C₁-C₃)alkyl group;
R6 represents a group selected from:
a phenyl group, said phenyl group being optionally substituted by 1 to 3 substituents independently selected from a halogen atom: a (C₁-C₆)alkyl group optionally substituted with a cyano group or a —OH group: a (C₁-C₆)fluoroalkyl group: a (C₃-C₆)cycloalkyl group; a (C₁-C₆)alkoxy group: a (C₁-C₆)fluoroalkoxy group: a cyano group; a trifluoromethylsulfonyl group: a (C₁-C₄)alkylthio group; a (C₁-C₄)fluoroalkylthio group: a (C₁-C₄)alkylsulfonyl group and a —OH group;
a fused phenyl group, selected from phenyl groups fused with a (C₃-C₆)cycloalkyl, said (C₃-C₆)cycloalkyl optionally comprises an unsaturation, and wherein the fused phenyl group is optionally substituted with 1 to 3 substituents independently selected from a (C₁-C₃)alkyl group, a hydroxy group, a halogen atom, a (C₁-C₆)fluoroalkyl group and a (C₁-C₃)alkoxy group;
a bicyclic group comprising 5 to 12 carbon atoms, optionally comprising 1 to 2 unsaturations; optionally substituted with 1 to 4 substituents independently selected from: a fluorine atom, a —OH group, a (C₁-C₃)alkyl group, a (C₁-C₃)fluoroalkyl group, a (C₁-C₃)alkoxy group, a (C₁-C₃)fluoroalkoxy group and an oxo group;
a heteroaryl group comprising 2 to 9 carbon atoms and comprising from 1 to 3 heteroatoms independently selected from oxygen, nitrogen and sulfur, and at least 5 atoms including carbon atoms and heteroatoms, said heteroaryl group being optionally substituted with 1 to 3 substituents independently selected from a halogen atom, a (C₁-C₆)alkyl group, a (C₁-C₆)fluoroalkyl group, a (C₁-C₆)alkoxy group, a (C₁-C₆)fluoroalkoxy group, a cyano group, a carbamoyl group and a —OH group;
a cycloalkyl group comprising 3 to 7 carbon atoms, said cycloalkyl group being saturated or partially saturated and being optionally substituted with 1 to 4 substituents independently selected from:
a fluorine atom, a —OH group, a (C₁-C₃)alkyl group, a (C₁-C₃)fluoroalkyl group, a (C₁-C₃)alkoxy group, a (C₁-C₃)fluoroalkoxy group, an oxo group,
a (C₃-C₆)cycloalkyl group and a phenyl group, said (C₃-C₆)cycloalkyl or phenyl groups being optionally substituted with one or two halogen atom(s) or (C₁-C₃)alkyl group(s);
a (C₃-C₆)cycloalkyl(C₁-C₃)alkyl group, optionally substituted on the cycloalkyl with 1 to 4 substituents independently selected from: a fluorine atom, a —OH group, a (C₁-C₄)alkyl group, a (C₁-C₃)fluoroalkyl group, a (C₁-C₃)fluoroalkoxy group and an oxo group;
a 4 to 7 membered-heterocycloalkyl group comprising 1 or 2 heteroatoms independently selected from oxygen, nitrogen and sulfur, said heterocycloalkyl group being saturated or partially saturated and being optionally substituted with 1 to 3 substituents independently selected from: a fluorine atom, a (C₁-C₃)alkyl group, a (C₁-C₃)fluoroalkyl group, a (C₁-C₃)fluoroalkoxy group, an oxo group, a (C₁-C₃)alkoxy group, and a —OH group;
a (C₁-C₆)alkyl group, said alkyl group being optionally substituted with 1 to 4 substituents independently selected from: a fluorine atom, a (C₁-C₃)alkoxy group, a (C₁-C₃)fluoroalkoxy group and a —OH group; and
a phenyl(C₁-C₂)alkyl group, said phenyl group being optionally substituted with 1 to 3 substituents independently selected from a halogen atom; a (C₁-C₃)alkyl group; a (C₁-C₃)fluoroalkyl group; a (C₁-C₃)alkoxy group; a (C₁-C₃)fluoroalkoxy group; a cyano group; and a —OH group;
X represents —CH₂—, —O— or —S—;
Y represents —CH═, —N═ or —CR″═, wherein R″ represents a (C₁-C₃)alkyl group, a halogen atom, a cyano group, or a (C₁-C₃)fluoroalkyl group;
R7 independently represents a (C₁-C₃)alkyl group, a halogen atom, a cyano group, or a (C₁-C₃)fluoroalkyl group;
R8 represents a hydrogen atom or a fluorine atom;
R9 represents a hydrogen atom, a (C₁-C₃)alkyl group or a cyclopropyl;
n is 0, 1 or 2; and
m is 0 or 1.

16. A compound of formula 1F or formula 1D, or a pharmaceutically acceptable salt thereof: 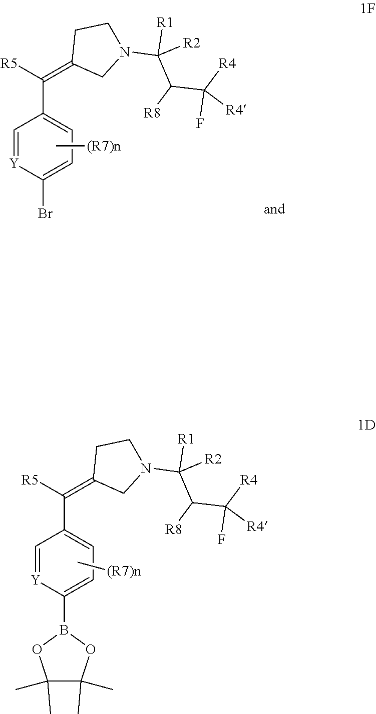
wherein:
R1 and R2 independently represent a hydrogen atom or a deuterium atom;
R4 and R4′ independently represent a hydrogen atom or a fluorine atom;
R5 represents a hydrogen atom, a fluorine atom or a (C₁-C₃)alkyl group;
R7 independently represents a (C₁-C₃)alkyl group, a halogen atom, a cyano group, or a (C₁-C₃)fluoroalkyl group;
R8 represents a hydrogen atom or a fluorine atom;
Y represents —CH═, —N═ or —CR″═, wherein R″ represents a (C₁-C₃)alkyl group, a halogen atom, a cyano group, or a (C₁-C₃)fluoroalkyl group; and
n is 0, 1 or 2.

17. (canceled)

18. A pharmaceutical composition, characterized in that it comprises a compound of formula (I) according to claim 1, or a pharmaceutically acceptable salt thereof, and at least one pharmaceutically acceptable excipient.

19. A method for inhibiting and degrading estrogen receptors, which comprises administering to a subject in need thereof a therapeutically effective amount of a compound of claim 1 or a pharmaceutically acceptable salt thereof.

20. A method of treating ovulatory dysfunction, cancer, endometriosis, osteoporosis, benign prostatic hypertrophy or inflammation, which comprises administering to a subject in need thereof a therapeutically effective amount of a compound of claim 1 or a pharmaceutically acceptable salt thereof.

21. The method according to claim 20, which comprises the treatment of cancer.
